(19) United States
(12) Patent Application Publication
(10) Pub. No.: US 2025/0237947 A1
(43) Pub. Date: Jul. 24, 2025

(54) Dry Film Resist, Photosensitive Dry Film, and Copper Clad Laminate

(71) Applicant: HANGZHOU FIRST ELECTRONIC MATERIAL CO., LTD, Hangzhou, Zhejiang (CN)

(72) Inventors: Li YUAN, Hangzhou, Zhejiang Province (CN); Gaohua ZHU, Hangzhou, Zhejiang Province (CN); Jiafeng WU, Hangzhou, Zhejiang Province (CN); Chuanlong HAN, Hangzhou, Zhejiang Province (CN); Weijie LI, Hangzhou, Zhejiang Province (CN); Weiqiang QIAN, Hangzhou, Zhejiang Province (CN)

(73) Assignee: HANGZHOU FIRST ELECTRONIC MATERIAL CO., LTD, Hangzhou, Zhejiang (CN)

(21) Appl. No.: 18/853,113

(22) PCT Filed: Mar. 20, 2023
(86) PCT No.: PCT/CN2023/082580

§ 371 Date: Sep. 30, 2024

(30) Foreign Application Priority Data
Apr. 2, 2022 (CN) 202210344255.4
Apr. 2, 2022 (CN) 202210346868.1
Apr. 2, 2022 (CN) 202210346902.5

(51) Int. Cl. G03F 7/031; C07D 231/06; C07D 401/14; C07D 403/14; C07D 409/04; G03F 7/033; G03F 7/09; G03F 7/11

(52) U.S. Cl. CPC G03F 7/031; C07D 231/06; C07D 401/14; C07D 403/14; C07D 409/04; G03F 7/033; G03F 7/094; G03F 7/11

(57) ABSTRACT

A dry film resist, a photosensitive dry film, and a copper clad laminate. The dry film resist includes an alkali-soluble resin (A), a photopolymerization monomer (B), a photoinitiator (C), and a sensitizer (D); and the sensitizer (D) includes a compound containing a pyrazoline structure.

### CROSS-REFERENCE TO RELATED APPLICATIONS

The present application is a National Stage of International Patent Application No: PCT/CN2023/082580 filed on Mar. 20, 2023, which claims the benefit of the priority of a Chinese patent application submitted to the Patent Office of the People's Republic of China on Apr. 2, 2022, with application No. 202210346868.1, 202210344255.4, and 202210346902.5, which is incorporated in the present application by reference in its entirety.

### TECHNICAL FIELD

The present disclosure relates to the field of optoelectronic materials, and specifically to a dry film resist, a photosensitive dry film, and a copper clad laminate.

### BACKGROUND

Since the introduction of a photosensitive resin composition, the photosensitive resin composition has become an important material in the modern electronics field, especially in the field of printed circuit boards (PCBs).

With the thin, light and short development of electronic devices, the PCBs are correspondingly required to be more highly refined, high-density, multilayered. The traditional photomask exposure method is large in consumption of film negatives and high in production cost, and a precision degree of line patterns obtained by the exposure method is limited, thus the exposure method is gradually being replaced by Laser Direct Imaging (LDI), which uses digital data to direct expose an active light image without the film negatives. Through the direct depiction exposure method, resist patterns can be formed with high productivity and high resolution. As a light source, an i ray (355 nm) or h ray (405 nm) is used. A laser with a wavelength being 405 nm is commonly used, which provides better exposure accuracy and may form high-density photosensitive patterns that are difficult to produce with previous technologies.

An Integrated Circuit (IC) is the core of the electronic device. An IC packaging substrate, which is also referred to as an IC carrier board, is a high-end PCB with higher routing density, is directly configured to carry a chip, and provides an electronic connection between the chip and a PCB motherboard.

In the past decade, due to the continuous increase in the annual production of smartphones and the constant need for iterative updating, on the other hand, with the increasing maturity of Semi-Additive (SAP), modified SAP (mSAP), Substrate-Like PCB (SLP) high-precision PCB manufacturing processes, the global market size of high-end PCBs such as carrier boards and SLPs is in a stage of extremely rapid growth. A few years ago, the research and development and production of the high-end PCBs such as carrier boards and SLPs are mainly concentrated in Japan and South Korea enterprises, but in recent years, some domestic enterprises have mastered the IC carrier board production technology, and the development is very rapid.

The IC carrier board is developed on the basis of a High Density Interconnector (HDI) board, but due to a smaller size of the packaging substrate and the more complexity of an electrical structure, the manufacturing technology is more difficult than HDIs and ordinary PCBs. Similar to the PCB manufacturing processes, the packaging substrate production process may be broadly divided into three categories of different production processes such as a subtractive method, an additive method, a semi-additive method, etc., and currently, the semi-additive method (mSAP) is a mainstream production process. The production process includes a plurality of procedures such as drilling, sinking, plating, pattern transfer, etching, solder resist, and coating. After the mSAP process is used, fine lines with a line width and line spacing being less than 25 μm may be produced, such that a problem of lateral erosion of lines manufactured by the subtractive method is effectively overcome. The mSAP process mainly depends on electroplating and flash corrosion. First, electroless copper plating is performed on a thin copper substrate, and a resist pattern is formed thereon, the pattern on the substrate is thickened through an electroplating process to remove the resist pattern; and then excess chemical copper layer is removed through flash corrosion, and the remaining portion is a formed line. Compared with the subtractive method, the width of the line is not affected by the thickness of copper plating, such that a higher analysis degree is achieved, and the line width and line spacing of the manufactured fine line are almost identical.

If the integration of the chip is higher, accordingly, the fineness and integration of the carrier board are getting higher. As a result, there are also higher requirements on the performance of a dry film photoresist used for the carrier board. A dry film resist for manufacturing the packaging substrate has extremely high requirements on aspects such as analysis precision and resist shapes, and is required to be able to form a resist pattern with a line width/spacing (L/S) of 10/10 μm or less.

In order to form the resist pattern with high precision, a photosensitizer generally needs to added to the photosensitive resin composition, and in the dry film resist for manufacturing the IC carrier boards and the SLPs, 9,10-dibutoxyanthracene (DBA) is, for example, added generally as the photosensitizer.

As described in a patent application with Publication No. of CN102272676A proposed by Hitachi Chemical Industry Co., Ltd., a dry film resist for manufacturing the packaging substrate has extremely high requirements on aspects such as analysis precision and resist shapes, and is required to be able to form a resist pattern with a line width/spacing (L/S) of 10/10 μm or less. In such high-order dry film resist, hexaarylbiimidazole derivatives with sensitizers such as pyrazoline, anthracene or triarylamine are commonly used in an initiator system. As shown in results in embodiments of the patent, by combining with suitable alkali-soluble resin and the photopolymerization monomer, the dry film resist may achieve excellent performance of aspects such as analysis degrees, adhesion, and resist shapes, but is extremely low in photosensibility, and even though exposure energy reaches 70 mJ/cm², the dry film resist has a sensitivity of only 11 frames. When the dry film resist with such low sensitivity is used, the production efficiency of PCB production clients is greatly affected.

On the other hand, the dry film resist is usually coated on the surface of a PET support film, a polyethylene film protective layer is tightly laminated to the surface after drying. As described in a patent application with Publication of No. CN113557474A, when the dry film resist contains the DBA, a phenomenon of infiltration of the DBA into a polyethylene film exists inevitably, causing problems of continuous attenuation of the sensitivity of the dry film resist and inability to form an expected pattern shape, and the infiltration problem is especially obvious when the protective layer is the polyethylene film.

Due to the adverse effect of light scattering on the analysis performance of the dry film resist, the analysis capacity of the dry film resist significantly reduces with increasing thickness. The analysis capacity of a traditional dry film resist is generally 0.8-1.0 time of the thickness of the dry film resist, and due to the process and precision requirements of the carrier boards and SLPs, the analysis capacity of the dry film resist used is required to reach below 0.5 times of a film thickness.

The film thickness of the dry film resist used for manufacturing existing carrier boards and SLPs is generally 20-29 um, the analysis capacity is required to be below 15 um, a 10 um/10 um line process is already on the daily agenda, and in the near future, the line precision of the IC carrier boards reaches 5 um/5 um.

Several initiator systems in the dry film resist reported in the related art and suitable for an LDI 405 nm exposure light source for PCB pattern transfer all have significant advantages, but also have obvious disadvantages. The current high-analysis and high-sensitivity dry film resist for manufacturing an HDI inner layer board generally uses acridine and derivatives thereof as the initiator system, although the acridine initiator is high in light sensitivity under an exposure light source with a wavelength being 405 nm, the initiator cannot promote the oxidization and color development of a leuco color developing agent after exposure, resulting in poor pattern comparison before and after exposure. A domestic LDI exposure machine cannot perform para-position recognition and cannot meet usage requirements for high photosensitivity and high precision PCB clients, and high para-position recognition adaptability of different types of LDI exposure machines.

On the other hand, with the thin, light and short development of electronic devices, the PCBs are correspondingly required to be more highly refined, high-density, multilayered. The traditional photomask exposure method is large in consumption of film negatives and high in production cost, and a precision degree of line patterns obtained by the exposure method is limited, thus the exposure method is gradually being replaced by Laser Direct Imaging (LDI), which uses digital data to direct expose an active light image without the film negatives. Through the direct depiction exposure method, resist patterns can be formed with high productivity and high resolution. The exposure light source generally uses a laser with a wavelength being 405 nm, which provides better exposure accuracy and may form high-density photosensitive patterns that are difficult to produce with previous technologies.

Vehicle PCBs account for a large percentage of total PCB demand, and the volume is continuing to grow, and for the manufacturing of PCBs requiring thick copper plates, such as automotive PCBs and industrial control boards, a plating manufacturing process is often used. In the past, an exposure mode corresponding to the PCB process is the traditional photomask exposure method. The exposure light source is UV mercury lamp irradiation or an LED light source.

However, in recent years, PCB manufacturing clients are gradually replacing the traditional photomask exposure method with laser LDI in order to improve production automation, efficiency and precision.

In order to meet the above requirements for manufacturing client process optimization of such PCBs, for the dry film resist, LDI laser light source with the wavelength being 405 nm needs to have high photosensitivity and excellent electroplating resistance at the same time.

A patent with Publication No. of CN101802710 has reported that, although the obtained dry film resist is better in electroplating resistance, the photosensitivity to the LDI with the wavelength being 405 nm is very poor. Since the initiator system used in the patent is the same as a commonly-used initiator in a conventional dry film resist, 4,4′-Bis(diethylamino) benzophenone is used as the initiator, and the photosensitivity of the initiator to the laser light source of 405 nm is severely insufficient, and thus cannot be used in an LDI dry film resist.

To sum up, there is still a need for improvement in a DBA-containing dry film resist resin composition in order to obtain a dry film resist with relatively-stable sensitivity and to obtain a resist pattern with higher precision. Therefore, in order to obtain more stable and higher precision comprehensive performance, for the dry film resist used for manufacturing high precision carrier boards, further optimization of the initiator system is an urgent problem to be solved.

### SUMMARY

The present disclosure is mainly intended to provide a dry film resist, a photosensitive dry film, and a copper clad laminate, so as to solve the problem that the performance of an existing dry film resist needs to be improved.

In order to implement the above objective, one aspect of the present disclosure provides a dry film resist. The dry film resist includes an alkali-soluble resin (A), a photopolymerization monomer (B), a photoinitiator (C), and a sensitizer (D); and the sensitizer (D) includes a compound containing a pyrazoline structure.

Further, the sensitizer (D) includes a compound containing an anthracene-substituted and/or triarylamine-substituted pyrazoline structure.

Preferably, the sensitizer (D) includes any one or more of the following compounds shown in structural formulas D1, D2, D3, D4, and D5.

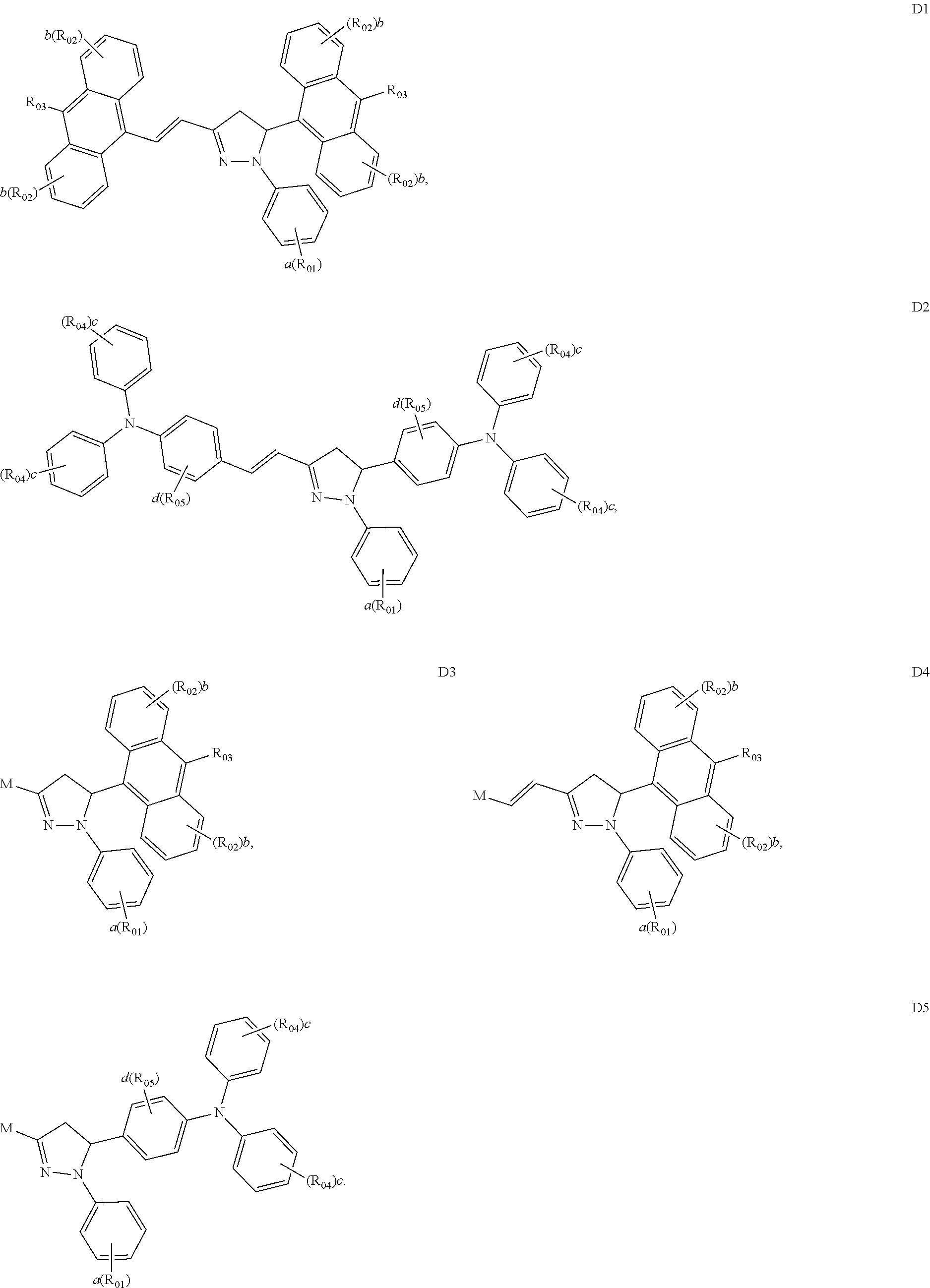

R₀₁, R₀₂, R₀₃, R₀₄, R₀₅ are independently any one or more of hydrogen, halogen, nitro, C₁-C₈ alkyl, and C₁-C₄ alkoxy, respectively.

a represents any one of integers among 0-5, b represents any one of integers among 0-4, c represents any one of integers among 0-5, d represents any one of integers among 0-4, and when a is greater than or equal to 2, a plurality of existing R₀₁s are able to be the same or different, respectively; when b is greater than or equal to 2, a plurality of existing R₀₂s are able to be the same or different, respectively; when c is greater than or equal to 2, a plurality of existing R₀₄s are able to be the same or different, respectively; when d is greater than or equal to 2, a plurality of existing R₀₅s are able to be the same or different, respectively.

Ms are Independently any One of Phenyl, Biphenyl, and a Condensed Ring Group Respectively, or an Electron-Rich Heterocyclic or Fused-Heterocyclic Group;

Preferably, the M is phenyl, biphenyl or polycyclic group with any one or more of C₁-C₄ alkoxy, amino, and alkyl, or is a heterocyclic or fused-heterocyclic group with furan, thiophene, indole, thiazole, benzofuran, benzothiazole, or fluorene.

More Preferably, the Sensitizer Includes Compounds Having the Following Structures,

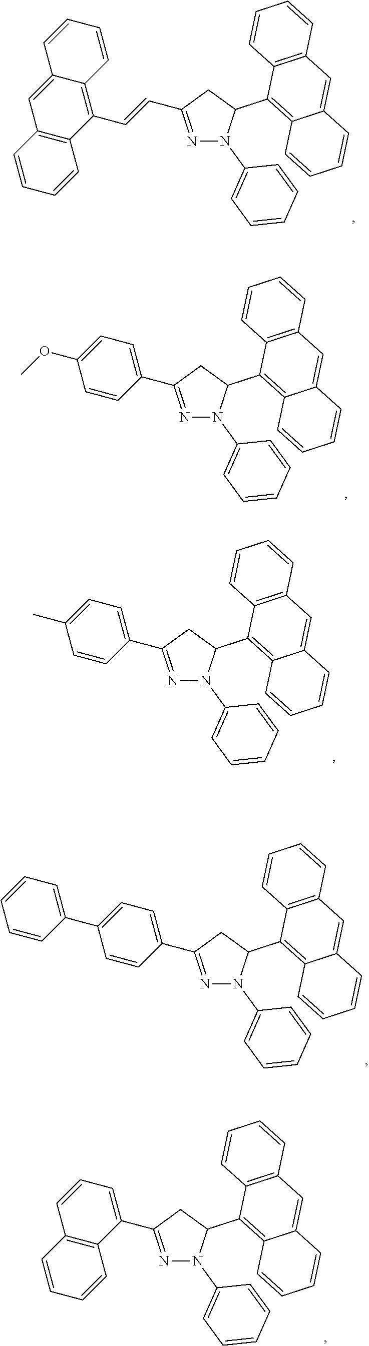

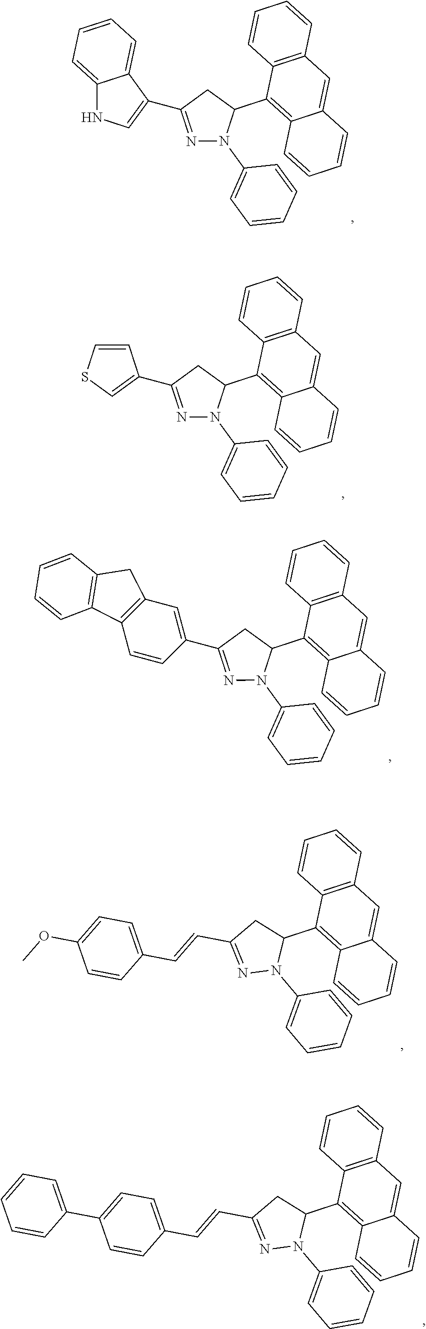 [SMILES: c1ccc(N2N=C(c3c[nH]c4ccccc34)CC2c2c3ccccc3cc3ccccc23)cc1 ; c1ccc(N2N=C(c3ccsc3)CC2c2c3ccccc3cc3ccccc23)cc1 ; c1ccc(N2N=C(c3ccc4c(c3)Cc3ccccc3-4)CC2c2c3ccccc3cc3ccccc23)cc1 ; COc1ccc(/C=C/C2=NN(c3ccccc3)C(c3c4ccccc4cc4ccccc34)C2)cc1 ; C(=C\c1ccc(-c2ccccc2)cc1)/C1=NN(c2ccccc2)C(c2c3ccccc3cc3ccccc23)C1]

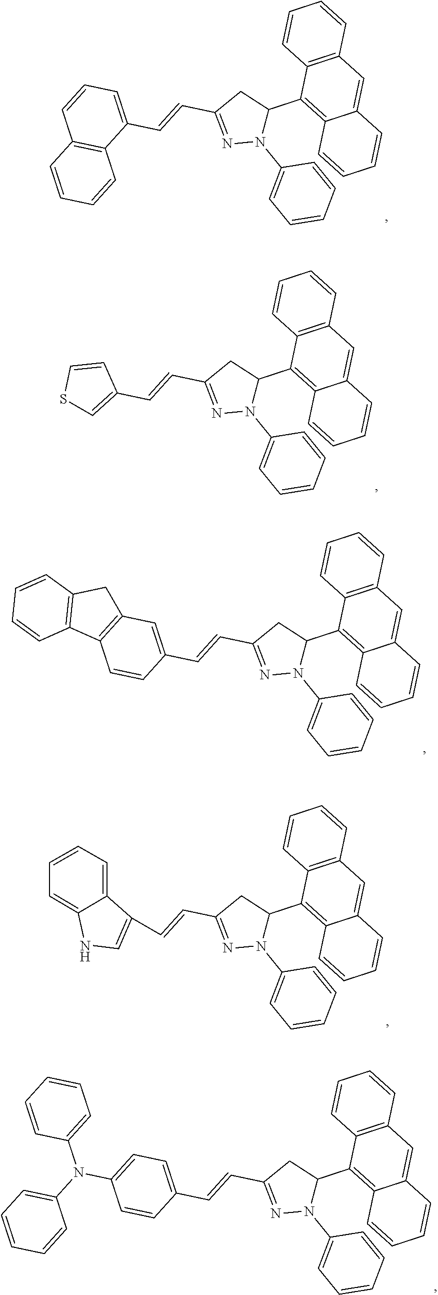 [SMILES: C(=C\c1cccc2ccccc12)/C1=NN(c2ccccc2)C(c2c3ccccc3cc3ccccc23)C1 ; C(=C\c1ccsc1)/C1=NN(c2ccccc2)C(c2c3ccccc3cc3ccccc23)C1 ; C(=C\c1ccc2c(c1)Cc1ccccc1-2)/C1=NN(c2ccccc2)C(c2c3ccccc3cc3ccccc23)C1 ; C(=C\c1c[nH]c2ccccc12)/C1=NN(c2ccccc2)C(c2c3ccccc3cc3ccccc23)C1 ; C(=C\c1ccc(N(c2ccccc2)c2ccccc2)cc1)/C1=NN(c2ccccc2)C(c2c3ccccc3cc3ccccc23)C1]

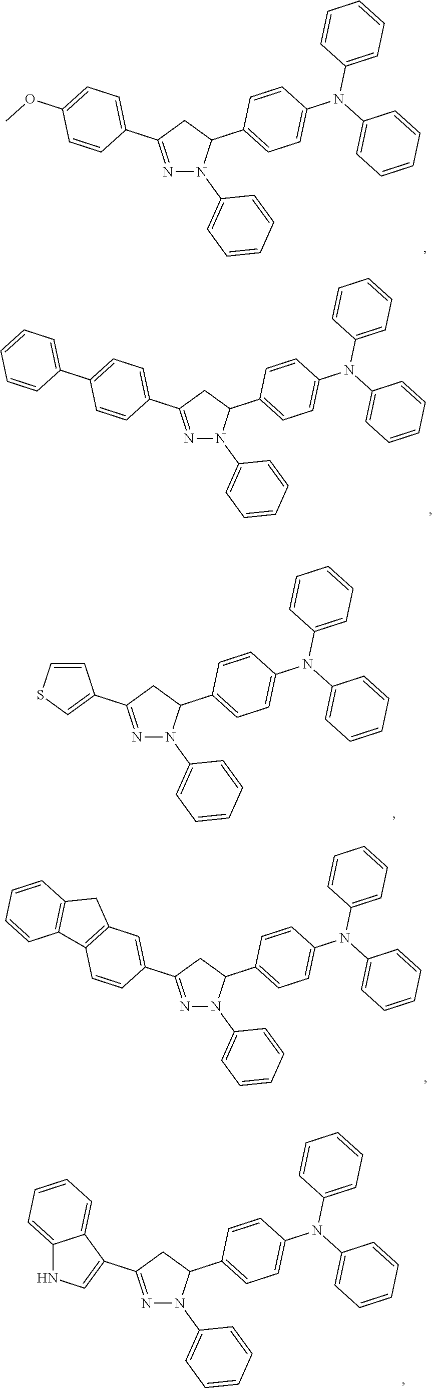 [SMILES: COc1ccc(C2=NN(c3ccccc3)C(c3ccc(N(c4ccccc4)c4ccccc4)cc3)C2)cc1 ; c1ccc(-c2ccc(C3=NN(c4ccccc4)C(c4ccc(N(c5ccccc5)c5ccccc5)cc4)C3)cc2)cc1 ; c1ccc(N(c2ccccc2)c2ccc(C3CC(c4ccsc4)=NN3c3ccccc3)cc2)cc1 ; c1ccc(N(c2ccccc2)c2ccc(C3CC(c4ccc5c(c4)Cc4ccccc4-5)=NN3c3ccccc3)cc2)cc1 ; c1ccc(N(c2ccccc2)c2ccc(C3CC(c4c[nH]c5ccccc45)=NN3c3ccccc3)cc2)cc1]

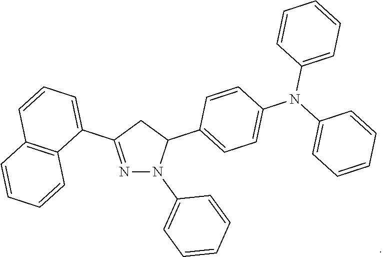 [SMILES: c1ccc(N(c2ccccc2)c2ccc(C3CC(c4cccc5ccccc45)=NN3c3ccccc3)cc2)cc1]

Optionally, the benzene ring, biphenyl ring, fused ring, or heterocyclic ring in the above structures contains a substituent, the substituent is any one or more of halogen, C₁-C₅ alkyl, and C₁-C₄ alkoxy, and preferably, the substituent is located in a para-position.

Further, the sensitizer (D) includes any one or more of the following compounds shown in a Structural formula I or II.

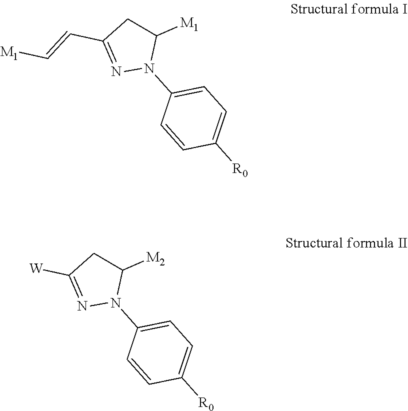

R₀s are independently hydrogen, halogen, C₁-C₈ alkyl, and C₁-C₄ alkoxy, respectively; modified groups M₁s in the Structural formula I are independently a biphenyl group, a condensed ring group, or electron-rich heterocycle, fused electron-rich heterocycle, or a benzene ring with any one or more of C₁-C₄ alkoxy and amino substituents; a modified group M₂ in the Structural formula II is selected from biphenyl, a condensed ring group, or electron-rich heterocycle, fused electron-rich heterocycle, or a benzene ring with any one or more of C₁-C₄ alkoxy, C₁-C₃ alkyl and amino substituents; and a modified group W is any one of a benzene ring and a fluorene ring or is a benzene ring, a biphenyl ring, or a fluorene ring with any one or more of halogen, C₁-C₈ alkyl, and C₁-C₄ alkoxy substituents.

Preferably, the modified groups M₁s are independently condensed ring groups with any one or more of C₁-C₄ alkoxy, C₁-C₄ amino, and C₁-C₄ alkyl respectively, or are any one of furan, thiophene, indole, thiazole, benzofuran, benzothiazole, indene, anthracene, acridine, and aromatic amine.

Preferably, a Specific Structural Formula of the Modified Group M₁ and/or M₂ Includes any One of

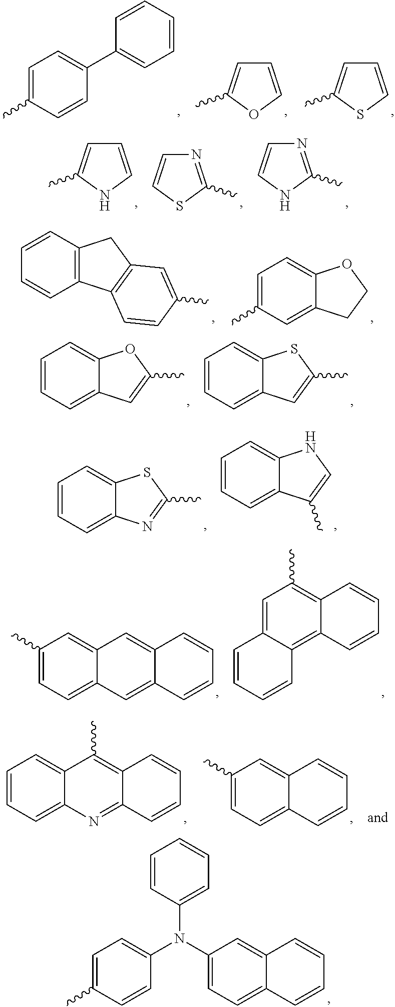

wherein [symbol] is a binding position of a group; and optionally, the benzene ring, biphenyl ring, fused ring, or heterocyclic ring on the specific structural formula of the modified group M₁ and/or M₂ contains any one or more of halogen, C₁-C₈ alkyl, and C₁-C₄ alkoxy substituents, and preferably, the substituent is located in a para-position.

Further, the dry film resist includes, by weight, 45-65 parts of the alkali-soluble resin (A), 30-50 parts of the photopolymerization monomer (B), 1.0-5.0 parts of the photoinitiator (C), and 0.01-0.5 parts of the sensitizer (D). Preferably, the dry film resist includes, by weight, 45-65 parts of the alkali-soluble resin (A), 30-50 parts of the photopolymerization monomer (B), 2.0-5.0 parts of the photoinitiator (C), and 0.01-0.5 parts of the sensitizer (D). Preferably, the content of the sensitizer is 0.01 wt %-0.5 wt % of a total weight of the dry film resist.

Further, the Photoinitiator (C) Includes the Following Compound Shown in a Structural Formula C1.

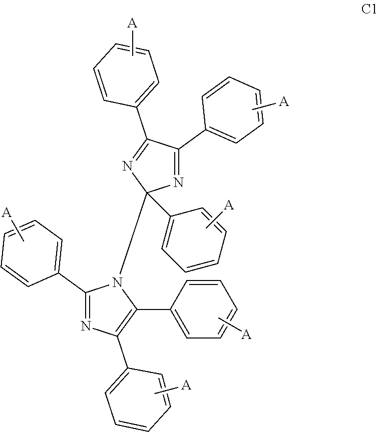

Substituents A on the benzene rings are independently any one or more of hydrogen, methoxy, and halogen atoms, respectively.

Preferably, the photoinitiator (C) includes one or more selected from a 2-(o-chlorophenyl)-4,5-diphenylimidazole dimer, a 2-(o-chlorophenyl)-4,5-di(methoxyphenyl)imidazole dimer, a 2-(o-fluorophenyl)-4,5-diphenylimidazole dimer, a2-(o-methoxyphenyl)-4,5-diphenylimidazole dimer, a 2-(p-methoxyphenyl)-4,5-diphenylimidazole dimer, and 2,2′,4-tris(2-chlorophenyl)-5-(3,4-dimethoxyphenyl)-4′,5′-diphenyl-1,1′-biimidazole.

Further, the dry film resist further includes a copper complex (E). A compound that forms a complex with copper in the copper complex (E) includes a nitrogen-containing heterocycle compound, the photopolymerization monomer (B) contains an EO chain segment and a PO chain segment, the EO chain segment represents oxoethylidene, and the PO chain segment represents oxypropylidene. Preferably, the copper complex (E) is 0.01-0.5 parts by weight. Preferably, the compound that forms the complex with copper in the copper complex (E) contains nitrogen heterocycle and sulfydryl at the same time. More preferably, the compound that forms the complex with copper in the copper complex (E) has any one or more of the following structures shown in a Structural formula III or Structural formula IV.

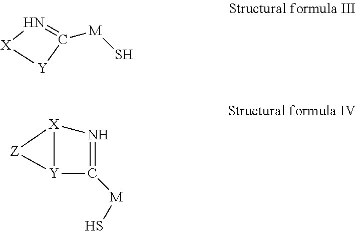

In the Structural formula III or Structural formula IV, X is 1-3 carbon atoms, or 1-2 nitrogen atoms, or one carbon atom and one nitrogen atom, and the carbon atoms and/or nitrogen atoms are linked by single bonds or double bonds; Y is selected from one or more of an oxygen atom, a sulfur atom, a carbon atom, a nitrogen atom, and the hydrogen atom on a ring formed by the X, the Y and —C═NH— is able to be substituted by any one or more of carboxyl, amino, C₁-C₁₂ alkyl, C₁-C₁₂ alkoxy, C₆-C₁₂ aryl, and hydrazinyl; M is selected from a single bond, C₁-C₁₂ alkyl, a C₁-C₁₂ ester group, or a C₂-C₁₂ ether group.

In the Structural formula IV, a ring formed by X, Y, and Z is a benzene ring or a heterocyclic ring, and the benzene ring and the heterocyclic ring are able to carry any one or more of C₁-C₆ alkyl, C₁-C₆ alkoxy, amino, carboxylic acid, nitro, and halogen.

Further preferably, the compound that forms the complex with copper in the copper complex (E) is selected from any one or more of mercaptopyrimidine, 4,6-diamino-2-mercaptopyrimidine, mercaptoimidazole, mercaptobenzimidazole, 2-mercapto-5-carboxybenzimidazole, 2-mercapto-5-nitrobenzimidazole, 5-amino-2-benzimidazolethiol, 2-mercapto-1H-benzo[d]iMidazole-4-carboxylic acid, mercaptobenzothiazole, 3-mercaptoindole, 1,3,5-tris(thioethyl)-1,3,5-triazine-2,4,6-trione, mercaptopurine, 6-thioguanine, trithiocyanuric acid, 2,6-dithiopurine, 4-thiouracil, 2-mercaptobenzoxazole, 4,6-diamino-2,6-mercaptopyrimidine, 4-thiouracil, 2-mercapto-4-hydroxy-5,6-diaminopyrimidine, 4,6-dimethyl-2-mercaptopyrimidine, dithiouracil, 2-mercaptopyrazine, 3,6-dimercaptopyridazine, 2-mercaptoimidazole, 2-mercaptothiazole, 8-mercaptoadenine, 4-thiouracil, mercaptotriazole, a 3-mercapto-1,2,4-triazole disulfide, 3-amino-5-mercapto-1,2,4-triazole, 4-methyl-i1,2,4-triazole-3-thiol, 3-amino-5-mercapto-1,2,4-triazole, 3-mercapto-1,2,4-triazole, a 6,7-dihydro-6-mercapto-5H-pyrazolo[1,2-A][1,2,4]triazole chloride, 4-amino-3-hydrazino-1,2,4-triazol-5-thiol, 5-mercapto-1-methyltetrazole, 2-(5-mercaptotetrazole-1-yl)ethanol, 1-[2-(dimethylamino)ethyl]-1H-tetrazole-5-thiol, 1-phenyltetrazole-5-thiol, 1,2-dihydro-1-(4-methoxyphenyl)-5H-tetrazole-5-thione, 1-ethyl-1H-1,2,3,4-tetrazole-5-thiol, 5-mercapto-1H-tetrazole-1-acetic acid, 5-mercapto-1H-tetrazole-1-methane sulphonic acid, N-[3-(5-mercapto-1H-1,2,3,4-tetraazol-1-yl)phenyl]acetamide, 1-(4-hydroxyphenyl)-2H-tetrazole-5-thione, 1-(4-ethoxyphenyl)-5-mercapto-1H-tetrazole, 1-(4-carboxyphenyl)-5-mercapto-1H-tetrazole, and 4-amino-2-mercaptopyrimidine.

Further, the Photopolymerization Monomer (B) Includes any One or More of the Following Compounds Shown in Structural Formulas B1, B2, and B3.

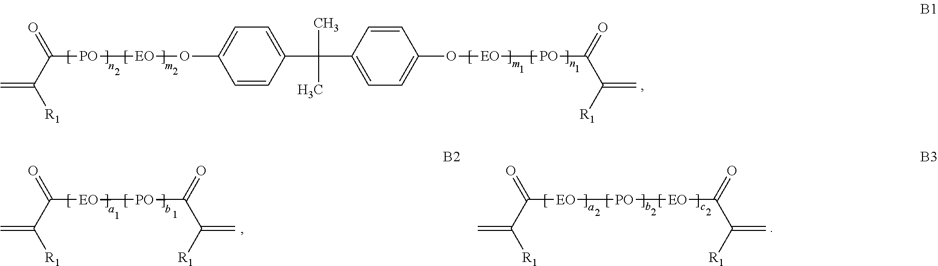

R₁s are independently H or CH₃ respectively, EO represents the oxoethylidene, PO represents the oxypropylidene, and EO and PO repeating units are arranged in a random or segmented manner; m₁ and m₂ are respectively any one of integers among 1-20, n₁ and n₂ are respectively any one of integers among 0-20, m₁+m₂ is any one of integers among 2-20, and n₁+n₂ is any one of integers among 0-20; a₁ is any one of integers among 4-20, and b₁ is any one of integers among 0-20; a₂ is any one of integers among 3-20, b₂ is any one of integers among 0-20, and c₂ is any one of integers among 3-20.

Preferably, a photopolymerization monomer having a structure shown in the structural formula B1 accounts for 40%-90% of a total amount of the photopolymerization monomer (B), further preferably accounts for 50%-80%, and is 20%-40% of a total weight of the photopolymerization monomer (B) and the alkali-soluble resin (A).

Further preferably, the photopolymerization monomer (B) further includes any one or more of structures shown in the structural formula B4.

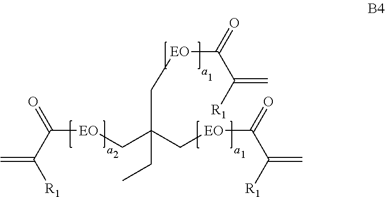

In the formula, R₁s are independently H or CH₃ respectively, and a₃s are independently any integers among 1-10 respectively.

More preferably, the photopolymerization monomer (B) is selected from any one or more of lauryl acrylate, lauryl methacrylate, octadecyl acrylate, octadecyl methacrylate, nonylphenol acrylate, isobornyl methacrylate, tetrahydrofurfuryl acrylate, bisphenol A diacrylate, bisphenol A dimethacrylate, poly ethylene glycol diacrylate, poly propylene glycol diacrylate, poly ethylene glycol dimethacrylate, poly propylene glycol dimethacrylate, ethoxylated neopentyl glycol diacrylate, propoxylated neopentyl glycol diacrylate, trimethylolpropane triacrylate, trimethylol propane trimethacrylate, ethoxylated trimethylolpropane trimethacrylate, propoxylated trimethylolpropane trimethacrylate, ethoxylated trimethylolpropane triacrylate, propoxylated trimethylolpropane triacrylate, ethoxylated pentaerythritol triacrylate, propoxylated pentaerythritol triacrylate, ethoxylated pentaerythritol tetraacrylate, propoxylated pentaerythritol tetraacrylate, ethoxylated dipentaerythritol pentaacrylate, propoxylated dipentaerythritol pentaacrylate, propoxylated dipentaerythritol hexaacrylate and ethoxylated dipentaerythritol hexaacrylate.

Further, the Photopolymerization Monomer (B) Includes any One or More of the Following Compounds Shown in the Structural Formula B1.

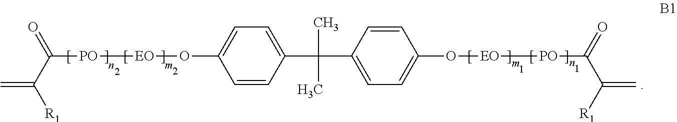

In the formula, R₁s are independently H or CH₃ respectively, m₁ and m₂ are respectively any one of integers among 1-20, n₁ and n₂ are respectively any one of integers among 0-20, m₁+m₂ is any one of integers among 2-20, n₁+n₂ is any one of integers among 0-20, EO represents the oxoethylidene, PO represents the oxypropylidene, and EO and PO repeating units are arranged in a random or segmented manner. Preferably, a photopolymerization monomer having a structure shown in the structural formula B1 accounts for 20%-80% of a total amount of the photopolymerization monomer, further preferably accounts for 40%-80%, and is 10%-35% of a total weight of the photopolymerization monomer (B) and the alkali-soluble resin (A).

Further preferably, the photopolymerization monomer (B) further includes one or more selected from lauryl acrylate, lauryl methacrylate, octadecyl acrylate, octadecyl methacrylate, nonylphenol acrylate, isobornyl methacrylate, tetrahydrofurfuryl acrylate, bisphenol A diacrylate, bisphenol A dimethacrylate, poly ethylene glycol diacrylate, poly propylene glycol diacrylate, poly ethylene glycol dimethacrylate, poly propylene glycol dimethacrylate, ethoxylated neopentyl glycol diacrylate, propoxylated neopentyl glycol diacrylate, trimethylolpropane triacrylate, trimethylolpropane trimethacrylate, ethoxylated trimethylolpropane trimethacrylate, propoxylated trimethylolpropane trimethacrylate, ethoxylated trimethylolpropane triacrylate, propoxylated trimethylolpropane triacrylate, pentaerythritol triacrylate, pentaerythritol tetraacrylate, dipentaerythritol pentaacrylate, and dipentaerythritol hexaacrylate.

Further, the Photopolymerization Monomer (B) Includes any One or More of the Following Compounds Shown in the Structural Formulas B1, B5, B6, and B7.

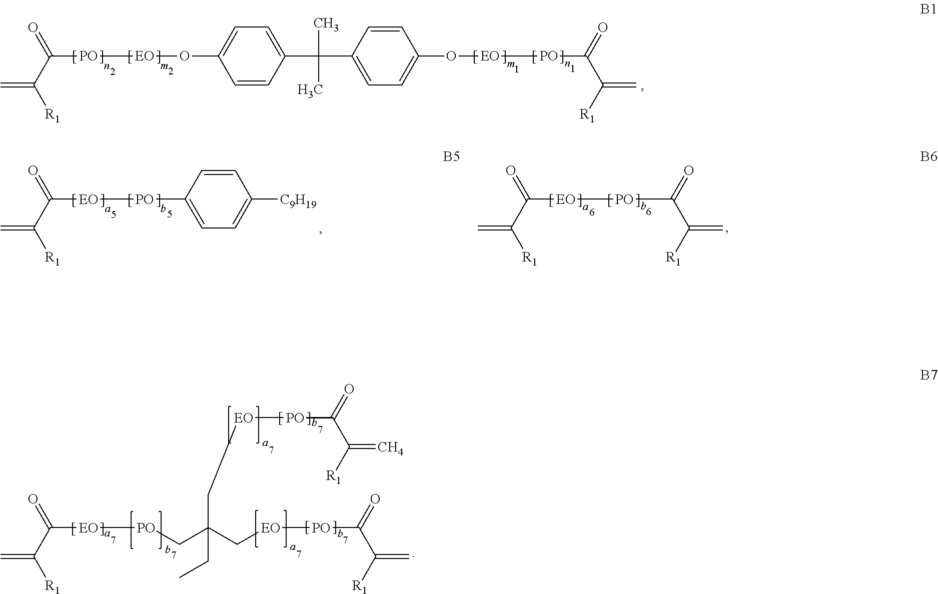

In the formula, R₁s are independently H or CH₃ respectively, and the EO chain segment and PO chain segment repeating units are arranged in a random or segmented manner; in the structural formula B1, m₁ and m₂ are respectively any one of integers among 0-30, n₁ and n₂ are respectively any one of integers among 0-20, m₁+m₂ is any one of integers among 0-30, and n₁+n₂ is any one of integers among 0-20; in the structural formula B5, a₅ is any integer among 0-30, and b₅ is any integer among 0-20; in the structural formula B6, a₆ is any integer among 0-30, b₆ is any integer among 0-20; in the structural formula B7, a₇ is any integer among 0-20, and b₇ is any integer among 0-20.

Preferably, a molar quantity of the PO chain segment is 15%-60% of a total molar quantity of the EO chain segment and the PO chain segment in the photopolymerization monomer.

Further preferably, the photopolymerization monomer (B) further includes any one or more selected from lauryl acrylate, lauryl methacrylate, octadecyl acrylate, octadecyl methacrylate, nonylphenol acrylate, isobornyl methacrylate, tetrahydrofurfuryl acrylate, bisphenol A dimethacrylate, bisphenol A diacrylate, poly ethylene glycol diacrylate, poly propylene glycol diacrylate, poly ethylene glycol dimethacrylate, poly propylene glycol dimethacrylate, ethoxylated neopentyl glycol diacrylate, propoxylated neopentyl glycol diacrylate, trimethylolpropane triacrylate, trimethylolpropane trimethacrylate, ethoxylated trimethylolpropane trimethacrylate, propoxylated trimethylolpropane trimethacrylate, ethoxylated trimethylolpropane triacrylate, propoxylated trimethylolpropane triacrylate, pentaerythritol triacrylate, pentaerythritol tetraacrylate, dipentaerythritol pentaacrylate, dipentaerythritol hexaacrylate, and polyurethaneacrylate.

Further, the alkali-soluble resin (A) is formed by copolymerizing methacrylic acid and/or acrylic acid, methacrylate and/or acrylate, and styrene or derivatives of the styrene, and the alkali-soluble resin (A) includes any one or more of the following compound shown in a structural formula A1.

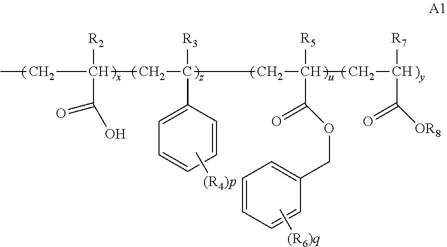

R₂, R₃, R₅, and R₇ are independently hydrogen or methyl respectively; R₄ and R₆ are independently any one or more of alkyl having 1-5 carbon atoms, alkoxy having 1-5 carbon atoms, hydroxyl or halogen atoms respectively; p and q independently represent any integer among 0-5 respectively; Rₐ is any one of straight chain, branched chain or cyclic alkyl having 1-18 carbon atoms; and x, y, z, and u respectively represent specific weights of various copolymerization components in the alkali-soluble resin, wherein x is 15-40 wt %, z is 0-50 wt %, u is 0-80 wt %, and y is 0-40 wt %.

Preferably, an acid value of the alkali-soluble resin (A) is 120-250 mg KOH/g, more preferably, a weight-average molecular weight is 30000-120000, a molecular weight distribution is 1.3-2.5, and further preferably, a polymerization conversion rate is greater than or equal to 97%.

Further, in the structural formula A1, x is 15-35 wt %, z is 0-50 wt %, u is 0-80 wt %, y is 0-25 wt %, and a value of z+u is 40 wt %-80 wt %.

Preferably, a copolymerization unit of the alkali-soluble resin (A) is selected from one or more of methyl methacrylate, methyl acrylate, ethyl methacrylate, ethyl acrylate, propyl methacrylate, propyl acrylate, isopropyl methacrylate, isopropyl acrylate, butyl methacrylate, butyl acrylate, isobutyl methacrylate, isobutyl acrylate, methylheptyl methacrylate, methylheptyl acrylate, dodecyl methacrylate, dodecyl acrylate, stearyl methacrylate, stearyl acrylate, 2-hydroxyethyl methacrylate, 2-hydroxyethyl acrylate, 2-hydroxypropyl methacrylate, 2-hydroxypropyl acrylate, 4-hydroxybutyl methacrylate, 4-hydroxybutyl acrylate, glycidyl methacrylate, glycidyl acrylate, N,N-dimethyl ethyl acrylate, N,N-dimethylethyl methacrylate, N,N-diethylethyl acrylate, N,N-diethylethyl methacrylate, N,N-diethylpropyl acrylate, N,N-diethylpropyl methacrylate, N,N-dimethylbutyl acrylate, N,N-dimethylbutyl methacrylate, N,N-diethylbutyl methacrylate and N,N-diethylbutyl acrylate.

Preferably, the weight-average molecular weight of the alkali-soluble resin (A) is 30000-80000, and the molecular weight distribution is 1.3-2.5.

Further, in the structural formula A1, R₃ is hydrogen, x is 15-40 wt %, z is 0-40 wt %, u is 0, and y is 20-60 wt %.

Preferably, a copolymerization unit of the alkali-soluble resin (A) is selected from alkyl methacrylate and/or alkyl acrylate and styrene and/or derivatives thereof; the alkyl methacrylate is selected from any one or more of methyl methacrylate, ethyl methacrylate, propyl methacrylate, isopropyl methacrylate, butyl methacrylate, isobutyl methacrylate, methylheptyl methacrylate, dodecyl methacrylate, stearyl methacrylate, 2-hydroxyethyl methacrylate, 2-hydroxypropyl methacrylate, 4-hydroxybutyl methacrylate, glycidyl methacrylate, N,N-dimethylethyl methacrylate, N,N-diethylethyl methacrylate, N,N-diethylpropyl methacrylate, N,N-dimethylbutyl methacrylate, and N,N-diethylbutyl methacrylate; the alkyl acrylate is selected from any one or more of methyl acrylate, ethyl acrylate, propyl acrylate, isopropyl acrylate, butyl acrylate, isobutyl acrylate, methylheptyl acrylate, dodecyl acrylate, stearyl acrylate, 2-hydroxyethyl acrylate, 2-hydroxypropyl acrylate, 4-hydroxybutyl acrylate, glycidyl acrylate, N,N-dimethylethyl acrylate, N,N-diethylethyl acrylate, N,N-diethylpropyl acrylate, N,N-dimethylbutyl acrylate, and N,N-diethylbutyl acrylate; the styrene derivatives are selected from one or more of a-methylstyrene, benzyl acrylate and benzyl methacrylate.

Further preferably, the content of the styrene in the copolymerization unit of the alkali-soluble resin (A) is 0-40 wt % of a total weight of the copolymerization unit.

Preferably, the weight-average molecular weight of the alkali-soluble resin (A) is 30000-80000, and the molecular weight distribution is 1.3-2.5.

Further, in the structural formula A1, R₃ is hydrogen, x is 15-40 wt %, z is 0-40 wt %, u is 0, and y is 20-70 wt %.

Preferably, the methacrylate is selected from any one or more of methyl methacrylate, ethyl methacrylate, propyl methacrylate, isopropyl methacrylate, butyl methacrylate, isobutyl methacrylate, methylheptyl methacrylate, dodecyl methacrylate, stearyl methacrylate, 2-hydroxyethyl methacrylate, 2-hydroxypropyl methacrylate, 4-hydroxybutyl methacrylate, glycidyl methacrylate, N,N-dimethylethyl methacrylate, N,N-diethylethyl methacrylate, N,N-diethylpropyl methacrylate, N,N-dimethylbutyl methacrylate, and N,N-diethylbutyl methacrylate; the acrylate is selected from any one or more of methyl acrylate, ethyl acrylate, propyl acrylate, isopropyl acrylate, butyl acrylate, isobutyl acrylate, methylheptyl acrylate, dodecyl acrylate, stearyl acrylate, 2-hydroxyethyl acrylate, 2-hydroxypropyl acrylate, 4-hydroxybutyl acrylate, glycidyl acrylate, N,N-dimethylethyl acrylate, N,N-diethylethyl acrylate, N,N-diethylpropyl acrylate, N,N-dimethylbutyl acrylate, and N,N-diethylbutyl acrylate. Preferably, the weight-average molecular weight of the alkali-soluble resin (A) is 50000-120000, and the molecular weight distribution is 1.3-2.5.

Further, the dry film resist further includes an additive. The additive includes any one or more of a free radical polymerization inhibitor, a color former, a coloring agent, a plasticizer, a photothermal stabilizer, an adhesion promoter, a leveling agent, and a defoaming agent, and preferably, the content of the additive is 0.5-5.0 parts by mass.

Further preferably, the content of the free radical polymerization inhibitor is 0.001 wt %-0.005 wt % of the total weight of the dry film resist.

Further preferably, the free radical polymerization inhibitor is selected from any one or more of 4-methoxyphenol, 4-ethyl-6-tert-butylphenol, nitroso phenylhydroxylamine aluminum salt, dimethyl-1,2-benzenediol, 3-methylcatechol, 4-methylcatechol, catechol, 2-ethylcatechol, 3-ethylcatechol, 4-ethylcatechol, 2-propylcatechol, 3-propylcatechol, 4-propylcatechol, 2-n-butylcatechol, 3-n-butylcatechol, 4-n-butylcatechol, 2-tert-butylcatechol, 3-tert-butylpyrocatechol, 4-tert-butylcatechol, 3,5-di-tert-butylcatechol, resorcinol, 2-methylresorcinol, 4-methylresorcinol, 5-orcinol, 2-ethylresorcinol, 4-ethylresorcinol, 2-propylresorcinol, 4-propylresorcinol, 2-n-butylresorcinol, 4-butylresorcinol, 2-tert-butylresorcinol, 4-tert-butylresorcinol, 1,4-hydroquinone, methylhydroquinone, ethylhydroquinone, propyl hydroquinone, tert-butylhydroquinone, 2,5-di-tert-butylhydroquinone, 2,6-di-tert-butyl-4-methylphenol, pyrogallol, 4-hydroxy-2,2,6,6-tetramethylpiperidine-N-oxyl, and 2,2-methylenebis(4-methyl-6-tert-butylphenol).

In order to implement the above objective, another aspect of the present application provides a photosensitive dry film. The photosensitive dry film includes a dry film resist layer, and a support layer and a protective layer located on two sides of the dry film resist layer. The dry film resist layer contains any one of the dry film resists described above.

Still another aspect of the present application provides a dry film resist. The dry film resist includes an alkali-soluble resin (A), a photopolymerization monomer (B), a photoinitiator (C), and a copper complex (E). A compound that forms a complex with copper in the copper complex (E) includes a nitrogen-containing heterocycle compound, the photopolymerization monomer (B) contains an EO chain segment and a PO chain segment, the EO chain segment represents oxoethylidene, and the PO chain segment represents oxypropylidene.

Further, the compound that forms the complex with copper in the copper complex (E) contains nitrogen heterocycle and sulfydryl at the same time; preferably, the compound that forms the complex with copper in the copper complex (E) has any one or more of the following structures shown in a Structural formula III or Structural formula IV.

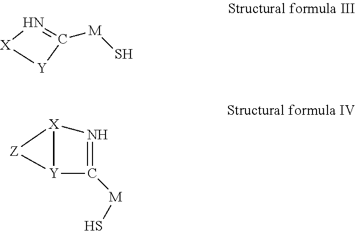

In the Structural formula III or Structural formula IV, X is 1-3 carbon atoms, or 1-2 nitrogen atoms, or one carbon atom and one nitrogen atom, and the carbon atoms and/or nitrogen atoms are linked by single bonds or double bonds; Y is selected from one or more of an oxygen atom, a sulfur atom, a carbon atom, and a nitrogen atom, and the hydrogen atom on a ring formed by the X, the Y and —C═NH— is able to be substituted by any one or more of carboxyl, amino, C₁-C₁₂ alkyl, C₁-C₁₂ alkoxy, C₆-C₁₂ aryl, and hydrazinyl; M is selected from a single bond, C₁-C₁₂ alkyl, a C₁-C₁₂ ester group, or a C₂-C₁₂ ether group.

In the Structural formula IV, a ring formed by X, Y, and Z is a benzene ring or a heterocyclic ring, and the benzene ring and the heterocyclic ring are able to carry any one or more of C₁-C₆ alkyl, C₁-C₆ alkoxy, amino, carboxylic acid, nitro, and halogen.

Further preferably, the compound that forms the complex with copper in the copper complex (E) is selected from any one or more of mercaptopyrimidine, 4,6-diamino-2-mercaptopyrimidine, mercaptoimidazole, mercaptobenzimidazole, 2-mercapto-5-carboxybenzimidazole, 2-mercapto-5-nitrobenzimidazole, 5-amino-2-benzimidazolethiol, 2-mercapto-1H-benzo[d]iMidazole-4-carboxylic acid, mercaptobenzothiazole, 3-mercaptoindole, 1,3,5-tris(thioethyl)-1,3,5-triazine-2,4,6-trione, mercaptopurine, 6-thioguanine, trithiocyanuric acid, 2,6-dithiopurine, 4-thiouracil, 2-mercaptobenzoxazole, 4,6-diamino-2,6-mercaptopyrimidine, 4-thiouracil, 2-mercapto-4-hydroxy-5,6-diaminopyrimidine, 4,6-dimethyl-2-mercaptopyrimidine, dithiouracil, 2-mercaptopyrazine, 3,6-dimercaptopyridazine, 2-mercaptoimidazole, 2-mercaptothiazole, 8-mercaptoadenine, 4-thiouracil, mercaptotriazole, a 3-mercapto-1,2,4-triazole disulfide, 3-amino-5-mercapto-1,2,4-triazole, 4-methyl-1,2,4-triazole-3-thiol, 3-amino-5-mercapto-1,2,4-triazole, 3-mercapto-1,2,4-triazole, a 6,7-dihydro-6-mercapto-5H-pyrazolo[1,2-A][1,2,4]triazole chloride, 4-amino-3-hydrazino-1,2,4-triazol-5-thiol, 5-mercapto-1-methyltetrazole, 2-(5-mercaptotetrazole-1-yl)ethanol, 1-[2-(dimethylamino)ethyl]-1H-tetrazole-5-thiol, 1-phenyltetrazole-5-thiol, 1,2-dihydro-1-(4-methoxyphenyl)-5H-tetrazole-5-thione, 1-ethyl-1H-1,2,3,4-tetrazole-5-thiol, 5-mercapto-1H-tetrazole-1-acetic acid, 5-mercapto-1H-tetrazole-1-methane sulphonic acid, N-[3-(5-mercapto-1H-1,2,3,4-tetraazol-1-yl)phenyl]acetamide, 1-(4-hydroxyphenyl)-2H-tetrazole-5-thione, 1-(4-ethoxyphenyl)-5-mercapto-1H-tetrazole, 1-(4-carboxyphenyl)-5-mercapto-1H-tetrazole, and 4-amino-2-mercaptopyrimidine.

Further, by weight, the dry film resist includes 40-65 parts of the alkali-soluble resin (A), 35-60 parts of the photopolymerization monomer (B), 2.0-4.5 parts of the photoinitiator (C), and 0.01-0.5 parts of the copper complex (E).

Further, the dry film resist further includes 0.01-0.5 parts of a sensitizer (D). Preferably, the sensitizer (D) includes any one or more of the following compounds shown in a Structural formula I or Structural formula II.

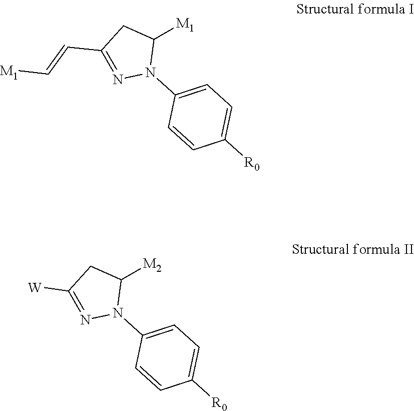

R₀s are independently hydrogen, halogen, C₁-C₈ alkyl, and C₁-C₄ alkoxy, respectively; modified groups M₁s in the Structural formula I are independently biphenyl, a condensed ring group or electron-rich heterocycle, fused electron-rich heterocycle, or a benzene ring with C₁-C₄ alkoxy or amino; a modified group M₂ in the Structural formula II is selected from any one of biphenyl, a condensed ring group or electron-rich heterocycle, fused electron-rich heterocycle, or a benzene ring with C₁-C₄ alkoxy or amino, or phenyl with C₁-C₄ alkoxy substituents; and a modified group W is any one of a benzene ring and a fluorene ring, or is a benzene ring, a biphenyl ring, or a fluorene ring with any one or more of halogen, C₁-C₈ alkyl, and C₁-C₄ alkoxy substituents.

Preferably, the modified groups M₁s are independently condensed ring groups with any one or more of C₁-C₄ alkoxy, C₁-C₄ amino, and C₁-C₄ alkyl respectively, or are any one of furan, thiophene, indole, thiazole, benzofuran, benzothiazole, indene, anthracene, acridine, and aromatic amine.

Further Preferably, a Specific Structural Formula of the Modified Group M₁ and/or M₂ Includes:

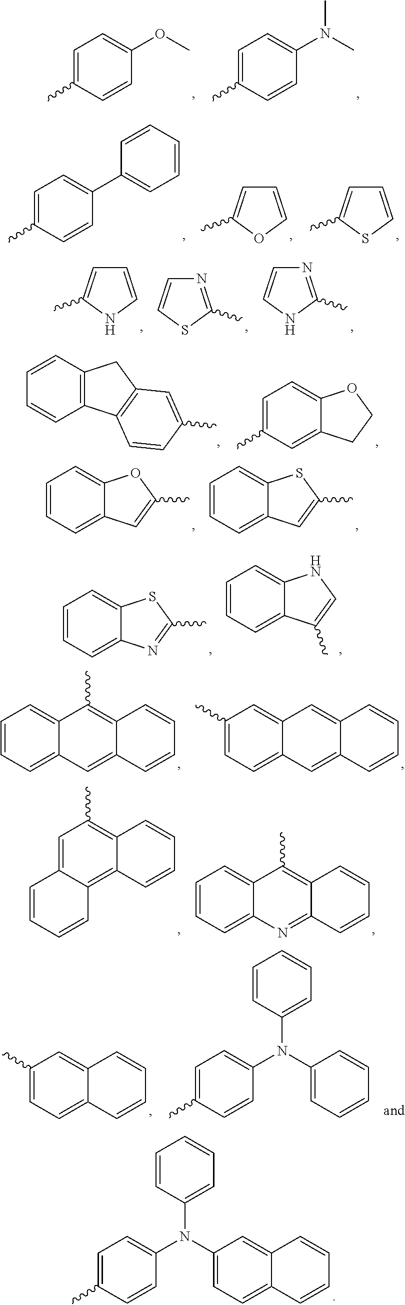

[symbol] is a binding position of a group. Optionally, the benzene ring, biphenyl ring, fused ring, or heterocyclic ring in the specific structural formula of the modified group M₁ and/or M₂ contains any one or more of substituents in halogen, C₁-C₅ alkyl, and C₁-C₄ alkoxy, and preferably, the substituent is located in a para-position.

Further, the Photoinitiator (C) Includes the Following Compound Shown in a Structural Formula

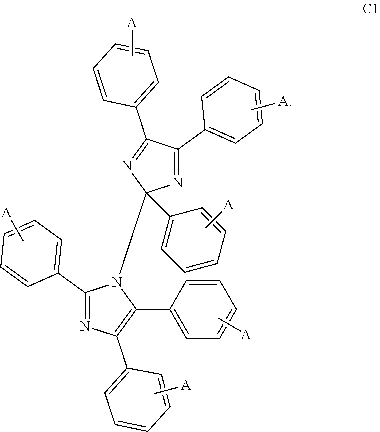

Substituents A on the benzene rings are independently hydrogen, methoxy, or halogen atoms.

Preferably, the photoinitiator (C) includes any one or more selected from a 2-(o-chlorophenyl)-4,5-diphenylimidazole dimer, a 2-(o-chlorophenyl)-4,5-di(methoxyphenyl)imidazole dimer, a 2-(o-fluorophenyl)-4,5-diphenylimidazole dimer, a 2-(o-methoxyphenyl)-4,5-diphenylimidazole dimer, a 2-(p-methoxyphenyl)-4,5-diphenylimidazole dimer, and 2,2′,4-tris(2-chlorophenyl)-5-(3,4-dimethoxyphenyl)-4′,5′-diphenyl-1,1′-biimidazole.

Optionally, the photoinitiator (C) further includes one or more selected from thioxanthone, benzoin phenyl ether, benzophenone, benzoin methyl ether, N,N′-tetramethyl-4,4′-diaminobenzophenone, N,N′-tetraethyl-4,4′-diaminobenzophenone, 4-methoxy-4′-dimethylamino diphenyl ketone, 2-benzyl-2-dimethylamino-1-(4-morpholinephenyl)-butanone, 2-ethyl anthraquinone, phenanthrenequinone, 2-tert-butylanthraquinone, octamethylanthraquinone, 1,2-benzanthraquinone, 2,3-benzoanthraquinone, 2,3-diphenylanthraquinone, 1-chloro anthraquinone, 2-methyl anthraquinone, 1,4-naphthoquinone, 9,10-phenanthrenequinone, 2,3-dimethylanthraquinone, benzoin methyl ether, benzoin ethyl ether, benzoin phenyl ether, 2,2-dimethoxy-2-phenylacetophenone, 9-phenylacridine, 1,7-bis(9,9′-acridinyl)heptane, anilinoacetic acid, a coumarin-based compound, and an oxazole-based compound.

Further, the photopolymerization monomer includes any one or more of the following compounds shown in the structural formulas B1, B5, B6, and B7.

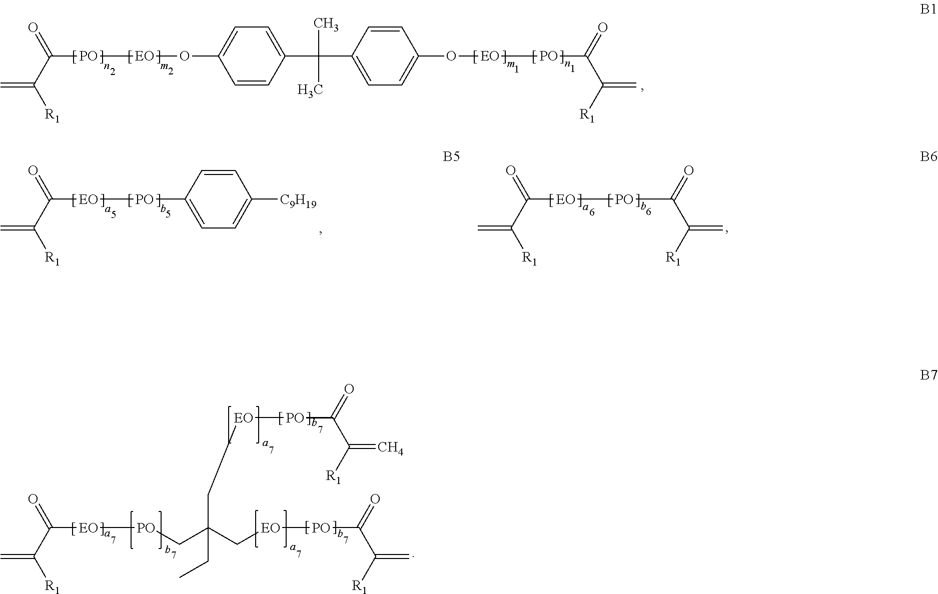

In the formula, R₁s are independently H or CH₃ respectively, and the EO chain segment and PO chain segment repeating units are arranged in a random or segmented manner; in the structural formula B1, m₁ and m₂ are respectively any one of integers among 0-30, n₁ and n₂ are respectively any one of integers among 0-20, m₁+m₂ is any one of integers among 0-30, and n₁+n₂ is any one of integers among 0-20; in the structural formula B5, a₅ is any integer among 0-30, and b₅ is any integer among 0-20; in the structural formula B6, a₆ is any integer among 0-30, b₆ is any integer among 0-20; in the structural formula B7, a₇ is any integer among 0-20, and b₇ is any integer among 0-20.

Preferably, a molar quantity of the PO chain segment is 15%-60% of a total molar quantity of the EO chain segment and the PO chain segment in the photopolymerization monomer.

Further preferably, the photopolymerization monomer (B) further includes any one or more selected from lauryl acrylate, lauryl methacrylate, octadecyl acrylate, octadecyl methacrylate, nonylphenol acrylate, isobornyl methacrylate, tetrahydrofurfuryl acrylate, bisphenol A dimethacrylate, bisphenol A diacrylate, poly ethylene glycol diacrylate, poly propylene glycol diacrylate, poly ethylene glycol dimethacrylate, poly propylene glycol dimethacrylate, ethoxylated neopentyl glycol diacrylate, propoxylated neopentyl glycol diacrylate, trimethylolpropane triacrylate, trimethylolpropane trimethacrylate, ethoxylated trimethylolpropane trimethacrylate, propoxylated trimethylolpropane trimethacrylate, ethoxylated trimethylolpropane triacrylate, propoxylated trimethylolpropane triacrylate, pentaerythritol triacrylate, pentaerythritol tetraacrylate, dipentaerythritol pentaacrylate, dipentaerythritol hexaacrylate, and polyurethane acrylate.

Further, the alkali-soluble resin (A) is formed by copolymerizing methacrylic acid and/or acrylic acid, methacrylate and/or acrylate, and styrene or derivatives of the styrene, and the alkali-soluble resin includes any one or more of the following structures shown in a structural formula A2.

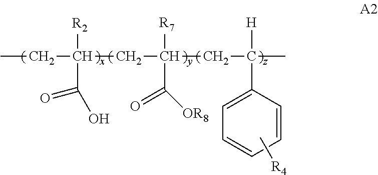

In the formula, R₂ and R₇ are independently hydrogen or methyl respectively; R₃ is selected from any one of C₁-C₁₈ straight chain alkyl, C₃-C₁₃ branched chain alkyl, benzyl, and C₁-C₁₈ straight chain alkyl or C₃-C₁₃ branched chain alkyl containing hydroxyl and/or amino substituents; R₄ is any one of C₁-C₃ alkyl, C₁-C₃ alkoxy, amino, and halogen atoms; the number of substituents on a benzene ring of the structural formula A2 is 0-5; x, y, and z respectively represent specific weights of various copolymerization components in the alkali-soluble resin, where x is 15-40 wt %, y is 20-70 wt %, and z is 0-40 wt %.

Preferably, the methacrylate is selected from any one or more of methyl methacrylate, ethyl methacrylate, propyl methacrylate, isopropyl methacrylate, butyl methacrylate, isobutyl methacrylate, methylheptyl methacrylate, dodecyl methacrylate, stearyl methacrylate, 2-hydroxyethyl methacrylate, 2-hydroxypropyl methacrylate, 4-hydroxybutyl methacrylate, glycidyl methacrylate, N,N-dimethylethyl methacrylate, N,N-diethylethyl methacrylate, N,N-diethylpropyl methacrylate, N,N-dimethylbutyl methacrylate, and N,N-diethylbutyl methacrylate; the acrylate is selected from any one or more of methyl acrylate, ethyl acrylate, propyl acrylate, isopropyl acrylate, butyl acrylate, isobutyl acrylate, methylheptyl acrylate, dodecyl acrylate, stearyl acrylate, 2-hydroxyethyl acrylate, 2-hydroxypropyl acrylate, 4-hydroxybutyl acrylate, glycidyl acrylate, N,N-dimethylethyl acrylate, N,N-diethylethyl acrylate, N,N-diethylpropyl acrylate, N,N-dimethylbutyl acrylate, and N,N-diethylbutyl acrylate. Further preferably, an acid value of the alkali-soluble resin is 120-250 mg KOH/g, more preferably, a weight-average molecular weight is 50000-120000, a molecular weight distribution is 1.3-2.5, and further preferably, a polymerization conversion rate is greater than or equal to 97%.

Further, the dry film resist further includes an additive. The additive includes any one or more of a color former, a coloring agent, a plasticizer, a photothermal stabilizer, an adhesion promoter, a leveling agent, a polymerization inhibitor, and a defoaming agent, and preferably, the content of the additive is 0.5-5.0 parts by weight.

Yet another aspect of the present application provides a photosensitive dry film. The photosensitive dry film includes a dry film resist layer, and a support layer and a protective layer located on two sides of the dry film resist layer. The dry film resist layer contains any one of the dry film resists described above.

Still another aspect of the present application provides a copper clad laminate. The copper clad laminate is provided with any one of the dry film resists described above.

Due to the use of a specific type of sensitizers, the dry film resist of the present application has good comprehensive performance, and is particularly outstanding in photosensitivity, resolution ratio, para-position recognition adaptability, and electroplating resistance.

### DETAILED DESCRIPTION OF THE EMBODIMENTS

It is to be noted that the embodiments in the present application and the features in the embodiments may be combined with one another without conflict. The present disclosure will be described below in detail with reference to the embodiments.

As analyzed in the Background of this application, a dry film resist in the related art is poor in photosensitivity and resolution ratio, and unstable in sensitivity and resist pattern. In order to solve the problems, the present application provides a dry film resist, a photosensitive dry film, and a copper clad laminate.

A typical implementation of the present application provides a dry film resist. The dry film resist includes an alkali-soluble resin (A), a photopolymerization monomer (B), a photoinitiator (C), and a sensitizer (D); and the sensitizer (D) includes a compound containing a pyrazoline structure.

The dry film resist includes the sensitizer containing a pyrazoline structure, which can effectively improve the comprehensive performance of the dry film resist in combined use with the alkali-soluble resin, the photopolymerization monomer, and the photoinitiator.

In some typical embodiments of the present application, the sensitizer (D) includes a compound containing an anthracene-substituted and/or triarylamine-substituted pyrazoline structure. In the present application, by introducing the anthracene and/or triarylamine in the pyrazoline structure, structure optimization is performed on high-order PCBs such as carrier boards and SLPs by using an anthracene and triarylamine sensitizer commonly-used in the dry film resist.

Since the imidazoline compound has good photosensitivity, the compound has higher photosensitivity to an LDI 405 nm laser light source than the anthracene and triarylamine sensitizer, the sensitizer compound with the above structure generally has a large π conjugated structure, and such large conjugated electron effect causes a red shift in a maximum absorption wavelength of a modified sensitizer compound to further approach the wavelength of the 405 nm laser light source, such that in the present application, by introducing the pyrazoline structure in particular anthracene and triarylamine molecules, the photosensitivity of the obtained dry film resist to the LDI 405 nm laser light source can be significantly improved.

The sensitizer structure simultaneously contains a pyrazoline ring structure and the anthracene or triarylamine structure, the optimized and modified sensitizer compound has a double active group, and the photosensitivity of the pyrazoline structure and the high-precision analysis capacity of the anthracene or triarylamine structure are effectively combined, such that the dry film resist containing same can improve the production efficiency of manufacturing high-order PCB clients such as carrier boards and SLPs while guaranteeing the high-precision analysis capacity.

Through analysis, the reason that the sensitizer DBA is susceptible to poor migration and penetration lies in that, on the one hand, the sensitivity of the DBA is very low, and the addition of the DBA required is relatively large in order to reach a certain curing degree to form a resist pattern with good shape; and on the other hand, DBA molecules are relatively small, and a molecular structure contains a long alkyl chain, such that according to molecular motion and similarity compatibility principles, a phenomenon of migration towards a polyethylene protective film having the same alkyl structure easily occurs in the DBA molecules. In the present application, according to the molecular motion mechanism and similarity compatibility principle, structure modification is performed on the anthracene sensitizer by introducing the pyrazoline structure into the anthracene compound, an introduced pyrazoline group generally carries other aromatic ring structures as well, thereby increasing a molecular weight of the anthracene sensitizer, and by introducing a large aromatic ring structure in the DBA molecular structure, the effects of the two aspects can both significantly suppress the poor migration and penetration of the sensitizer having the pyrazoline structure of the present application towards the polyethylene protective thin film, such that the dry film resist with more stable performance in terms of sensitivity, resist patterns, etc. can be obtained.

The compound containing the anthracene-substituted and/or triarylamine-substituted pyrazoline structure has a corresponding chemical structure, and thus may select a compound having the corresponding structure from existing compounds. In some preferred embodiments of the present application, the sensitizer (D) is any one or more of the following compounds shown in structural formulas D1, D2, D3, D4, and D5.

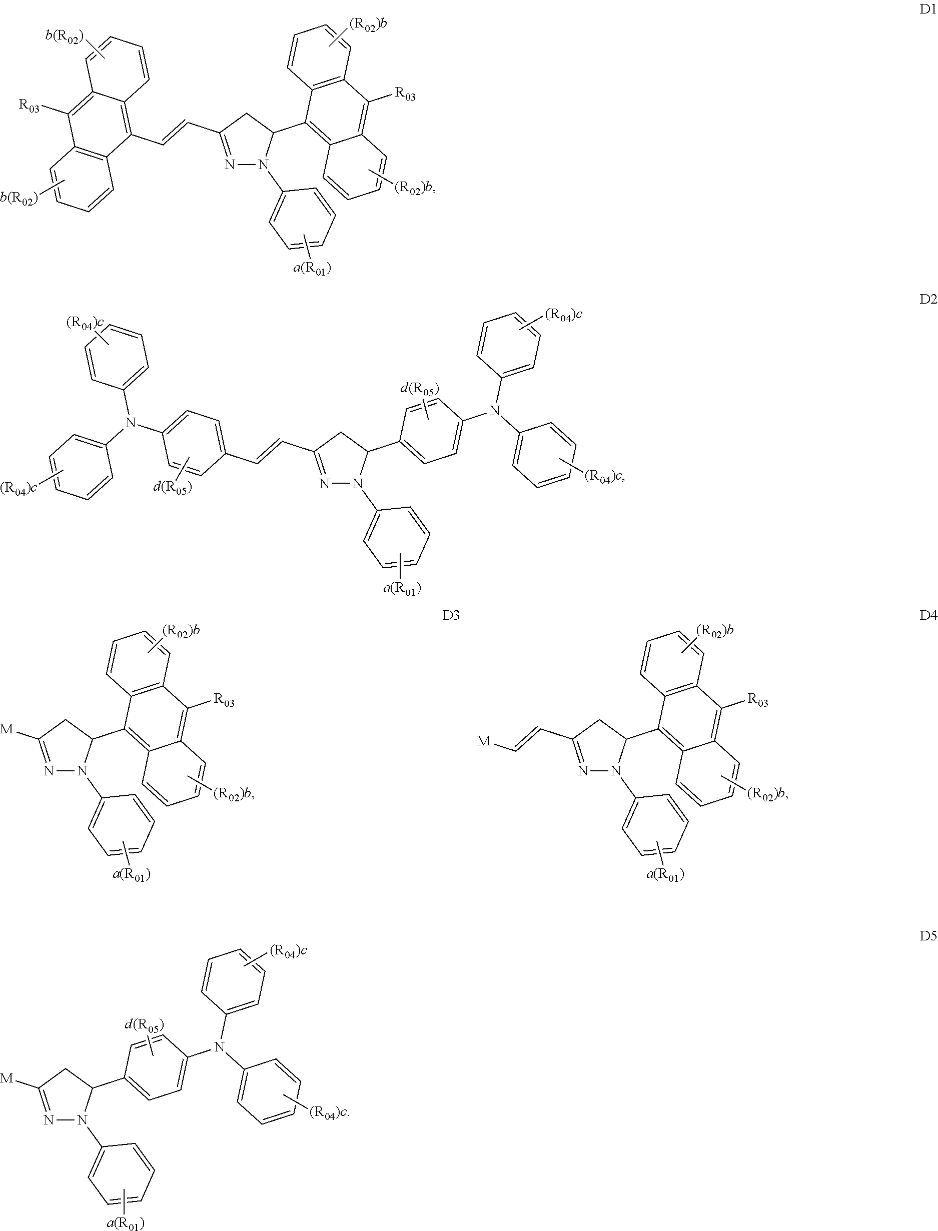

R₀₁, R₀₂, R₀₃, R₀₄, R₀₅ are independently any one or more of hydrogen, halogen, nitro, C₁-C₈ alkyl, and C₁-C₄ alkoxy, respectively.

a represents any one of integers among 0-5, b represents any one of integers among 0-4, c represents any one of integers among 0-5, d represents any one of integers among 0-4, and when a is greater than or equal to 2, a plurality of existing R₀₁s are able to be the same or different, respectively; when b is greater than or equal to 2, a plurality of existing R₀₂s are able to be the same or different, respectively; when c is greater than or equal to 2, a plurality of existing R₀₄s are able to be the same or different, respectively; when d is greater than or equal to 2, a plurality of existing R₀₅s are able to be the same or different, respectively. Ms are independently any one of phenyl, biphenyl, and a condensed ring group respectively, or an electron-rich heterocyclic or fused-heterocyclic group. The benzene ring represents a phenyl or phenyl derivative group; the biphenyl represents a biphenyl or biphenyl derivative group; the condensed ring group represents a condensed ring or condensed ring derivative group; and the electron-rich heterocyclic or fused-heterocyclic group represents an electron-rich heterocyclic or fused-heterocyclic derivative group. As an example, the above derivative group may be a group containing substituents such as C₁-C₄ alkoxy, amino, C₁-C₈ alkyl, halogen, etc., and there may be one or more substituents.

The Ms in the structural formulas D3, D4, and D5 may be selected from an existing benzene ring, biphenyl, and condensed ring group, or a benzene ring, biphenyl, and condensed ring group with electron-donating substituents, or the electron-rich heterocyclic or fused-heterocyclic group. In some embodiments of the present application, the M is a phenyl, biphenyl or polycyclic group with any one or more of C₁-C₄ alkoxy, amino, and alkyl, or is furan, thiophene, indole, thiazole, benzofuran, benzothiazole, or fluorene, and derivatives of the heterocyclic or fused-heterocyclic group. Preferably, the sensitizer includes compounds having the following structures:

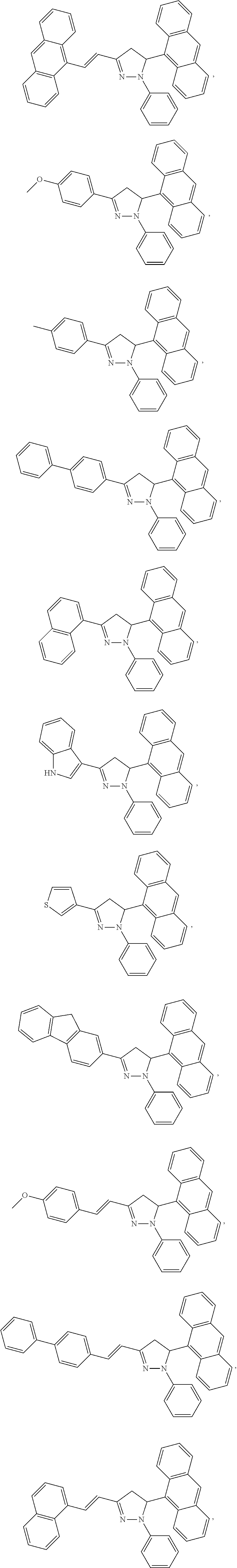

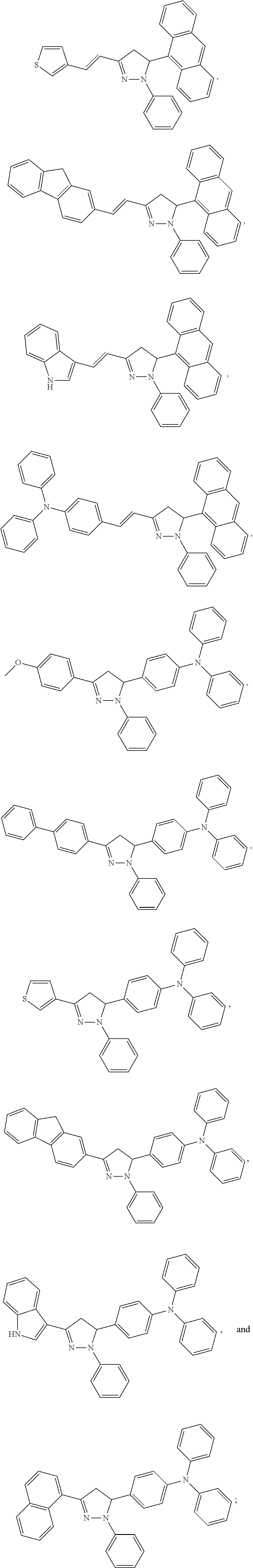

and the benzene ring, biphenyl ring, fused ring, or heterocyclic ring may also contain a substituent, the substituent is any one or more of halogen, C₁-C₈ alkyl, and C₁-C₄ alkoxy, and the number of substituents is not limited, preferably, the substituent is located in a para-position, thereby achieving excellent effects.

In Some Typical Embodiments of the Present Application, the Sensitizer (D) Includes any One or More of the Following Compounds Shown in a Structural Formula I or Structural Formula II.

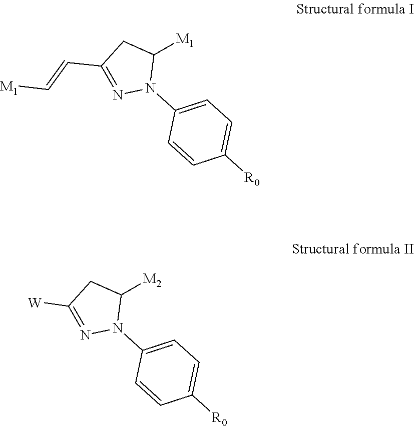

R₀s are independently hydrogen, halogen, C₁-C₈ alkyl, and C₁-C₄ alkoxy, respectively; modified groups M₁s in the Structural formula I are independently a biphenyl group, a condensed ring group, or electron-rich heterocycle, fused electron-rich heterocycle, or a benzene ring with any one or more of C₁-C₄ alkoxy and amino substituents; a modified group M₂ in the Structural formula II is selected from biphenyl, a condensed ring group or electron-rich heterocycle, fused electron-rich heterocycle, or a benzene ring with any one or more of C₁-C₄ alkoxy, C₁-C₃ alkyl and amino substituents; and a modified group W is any one of a benzene ring and a fluorene ring, or is a benzene ring, a biphenyl ring, or a fluorene ring with any one or more of halogen, C₁-C₈ alkyl, and C₁-C₄ alkoxy substituents.

In the present application, by introducing some modified groups such as biphenyl, condensed ring groups, electron-rich heterocycle or fused heterocycle, and the benzene ring with the amino into the pyrazoline compound, an entire molecular structure of the modified sensitizer compound shown in the Structura formulas I and Structura formulas II is an electron-rich conjugated system, such electron-rich conjugation effect facilitates the promotion of red shift of an absorption spectrum of the sensitizer, and the absorption spectrum may extend to a visible region. A maximum absorption wavelength of the modified sensitizer is closer to an exposure light source with a wavelength being 405 nm, which is more sensitive to the exposure light source, such that the photosensitivity of the dry film resist to an LDI 405 nm exposure light source is improved.

Furthermore, by selecting the sensitizer with the above particular, the dry film resist is high in photosensitivity and resolution ratio, and the sensitizer can effectively promote the oxidization and color development of a leuco color developing agent after exposure, such that a pattern contrast ratio before and after exposure can be further enhanced, thereby more facilitating para-position recognition of domestic LDI exposure machines, so as to guarantee para-position precision of the sensitizer on the domestic LDI exposure machines. In addition, the dry film resist of the present application containing the sensitizer is rich in component source, and in particular, the sensitizer can not only make the dry film resist have high photosensitivity and high para-position recognition adaptability, but also have rich sources, and thus is low in cost, such that the production cost of a high-end dry film sensitizer may be effectively reduced, thereby having good market application values.

The modified group M₁ and/or M₂ of the Structural formula I or Structural formula II may select any one or more of biphenyl, the condensed ring group or the electron-rich heterocycle, the fused electron-rich heterocycle, or the benzene ring with the amino. For example, the modified group M, and/or M₂ is independently the condensed ring group with any one or more of C₁-C₄ alkoxy, amino, and alkyl respectively, or is a heterocyclic or fused-heterocyclic group with any one of furan, thiophene, indole, thiazole, benzofuran, benzothiazole, indene, anthracene and acridine, and includes derivatives of the groups.

In some preferred embodiments, a specific structural formula of the modified group M₁ and/or M₂ includes any one of

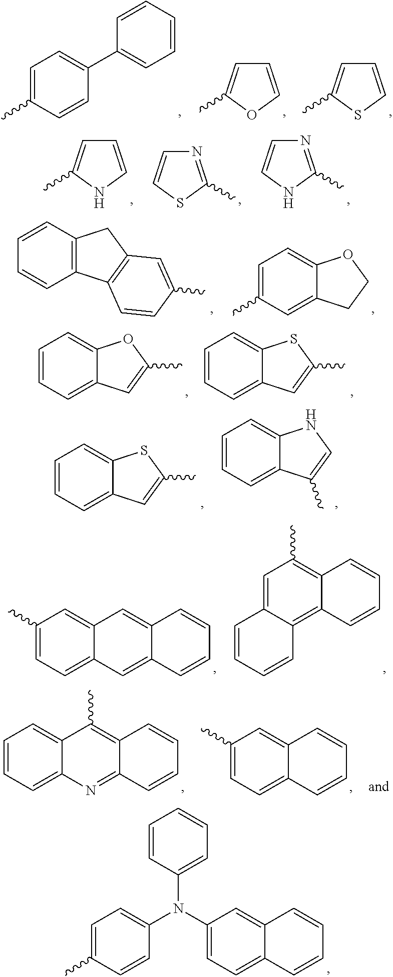

wherein [symbol] is a binding position of a group; and optionally, the benzene ring, biphenyl ring, fused ring, or heterocyclic ring on the specific structural formula of the modified group M₁ and/or M₂ contains any one or more of halogen, C₁-C₈ alkyl, and C₁-C₄ alkoxy substituents, and the number of substituents is not limited, preferably, the substituent is located in a para-position.

The contents of the alkali-soluble resin (A), the photopolymerization monomer (B), the photoinitiator (C), and the sensitizer (D) in the dry film resist of the present application may be determined according to existing technologies, and are not limited in the present application. In some embodiments of the present application, in order to better achieve the synergistic effect of each component, the dry film resist includes: by weight, 45-65 parts of the alkali-soluble resin (A), 30-50 parts of the photopolymerization monomer (B), 1.0-5.0 parts of the photoinitiator (C), and 0.01-0.5 parts of the sensitizer (D). Since the addition of the sensitizer is too low, the photosensitivity of the dry film resist is poor, and if the addition is too high, a curing depth is affected due to too fast curing of a surface layer of the dry film resist, causing a risk of reducing the adhesion of the dry film resist to a copper surface, such that, in order to improve the sensitivity degree, the resolution ratio, and the adhesion more evenly, the content of the sensitizer is preferably 0.01 wt %-0.5 wt % of the total weight of the dry film resist.

In some embodiments of the present application, in order to further improve the photosensitivity, resolution ratio, and sensitivity of the dry film resist and the stability of the resist patterns, the dry film resist includes, by weight, 45-65 parts of the alkali-soluble resin (A), 30-50 parts of the photopolymerization monomer (B), 2.0-5.0 parts of the photoinitiator (C), and 0.01-0.5 parts of the sensitizer (D).

The photoinitiator (C) may be selected from the existing technologies, such as a hexaarylbiimidazole derivative photoinitiator. In order to further improve the photosensitivity, resolution ratio, and adhesion of the dry film resist, in some embodiments of the present application, the photoinitiator (C) has the following compound shown in a structural formula C₁.

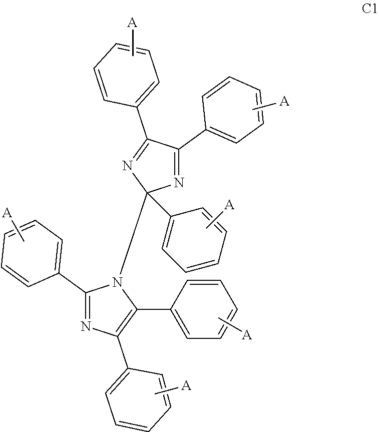

Substituents A on the benzene rings are independently any one or more of hydrogen, methoxy, and halogen atoms, respectively.

Preferably, the photoinitiator (C) is selected from one or more selected from a 2-(o-chlorophenyl)-4,5-diphenylimidazole dimer, a 2-(o-chlorophenyl)-4,5-di(methoxyphenyl)imidazole dimer, a 2-(o-fluorophenyl)-4,5-diphenylimidazole dimer, a 2-(o-methoxyphenyl)-4,5-diphenylimidazole dimer, a 2-(p-methoxyphenyl)-4,5-diphenylimidazole dimer, and 2,2′,4-tris(2-chlorophenyl)-5-(3,4-dimethoxyphenyl)-4′,5′-diphenyl-1,1′-biimidazole. While the photoinitiator is added, a small amount of other types of photoinitiators may also used together, for example, one or more of thioxanthone, benzoin phenyl ether, benzophenone, benzoin methyl ether, N,N′-tetramethyl-4,4′-diaminobenzophenone, N,N′-tetraethyl-4,4′-diaminobenzophenone, 4-methoxy-4′-dimethylamino diphenyl ketone, 2-benzyl-2-dimethylamino-1-(4-morpholinephenyl)-butanone, 2-ethyl anthraquinone, phenanthrenequinone, 2-tert-butylanthraquinone, octamethylanthraquinone, 1,2-benzanthraquinone, 2,3-benzoanthraquinone, 2,3-diphenylanthraquinone, 1-chloro anthraquinone, 2-methyl anthraquinone, 1,4-naphthoquinone, 9,10-phenanthrenequinone, 2,3-dimethylanthraquinone, benzoin methyl ether, benzoin ethyl ether, benzoin phenyl ether, 2,2-dimethoxy-2-phenylacetophenone, benzoyl derivatives such as benzoyl dimethyl ether, acridine derivatives such as 9-phenylacridine, 1,7-bis(9,9′-acridinyl)heptane, anilinoacetic acid, a coumarin-based compound, and an oxazole-based compound. The specific amount of the above photoinitiator may be determined according to the prior art, and is not limited herein.

In some typical embodiments of the present application, in order to improve the electroplating resistance of the dry film resist, any one of the above dry film resists further includes a copper complex (E). A compound that forms a complex with copper in the copper complex (E) includes a nitrogen-containing heterocycle compound, the photopolymerization monomer (B) contains an EO chain segment and a PO chain segment, the EO chain segment represents oxoethylidene, and the PO chain segment represents oxypropylidene. The EO chain segment and the PO chain segment may be from one or more photopolymerization monomers simultaneously containing the EO chain segment and the PO chain segment, or the photopolymerization monomers respectively containing the EO chain segment and the PO chain segment may also be compounded, or the photopolymerization monomers containing different ratios of the EO chain segment and the PO chain segment are compounded.

For the dry film resist containing nitrogen-containing heterocycle compound and the copper-formed complex, ne the one hand, due to large polarity of a nitrogen heterocyclic structure, the copper complex can improve an acting force with copper, and on the other hand, lone pair electrons on nitrogen atoms can coordinate and complex with copper atoms, and a binding force between the compound and the copper atoms can be further improved through the generation of such coordination chemical bonds. Through the effects of the above two aspects, the binding force between the dry film resist and a copper surface can be significantly improved by adding the copper complex compound, such that a phenomenon that a dry film slightly warps on a bottom side wall during a plating process is improved, thereby improving the electroplating resistance of the dry film resist.

The photopolymerization monomer simultaneously contains a hydrophilic group Ethylene Oxide (EO) and a hydrophobic group Propylene Oxide (PO). On the one hand, the photopolymerization monomer containing a hydrophilic group EO chain segment may improve the water-solubility of the dry film resist, such that the development performance and resolution ratio of the dry film resist are improved; and on the other hand, when the photopolymerization monomer only contains the EO chain segment, the water-solubility of the dry film resist is improved, the dry film resist is susceptible to dry film sidewall wrapping due to swelling, causing an undesirable phenomenon of cementation, in the photopolymerization monomer, by adding a hydrophobic group PO chain segment, the hydrophobicity of the dry film resist may be improved to a certain extent, thereby improving the electroplating resistance of the dry film resist. Therefore, the photopolymerization monomer chemically balances the hydrophilicity and hydrophobicity of the dry film resist, and a dry film resist photosensitive material with balanced development performance, analysis capacity, and electroplating resistance can be obtained.

In order to further improve the electroplating resistance of the dry film resist, in some embodiments of the present application, the copper complex (E) is 0.01-0.5 parts by weight. The nitrogen-containing heterocycle compound that forms the complex with copper has no special requirements, may be saturated nitrogen heterocycle, or may also be unsaturated nitrogen heterocycle. The heterocyclic ring may contain one nitrogen atom, or may also contain a plurality of nitrogen atoms. In some preferred embodiments of the present application, the compound that forms the complex with copper in the copper complex contains nitrogen heterocycle and sulfydryl at the same time; and the number of sulfydryl may be one or more.

In some preferred embodiments, the compound that forms the complex with copper in the copper complex (E) has any one of the following structures shown in a Structural formula III or Structural formula IV.

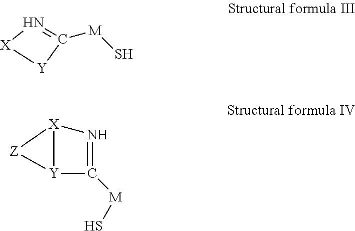

In the Structural formula III or Structural formula IV, X is 1-3 carbon atoms, or 1-2 nitrogen atoms, or one carbon atom and one nitrogen atom, and the carbon atoms and/or nitrogen atoms are linked by single bonds or double bonds; Y is selected from one or more of an oxygen atom, a sulfur atom, a carbon atom, a nitrogen atom, and the hydrogen atom on a ring formed by the X, the Y and —C═NH— is able to be substituted by any one or more of carboxyl, amino, C₁-C₁₂ alkyl, C₁-C₁₂ alkoxy, C₆-C₁₂ aryl, and hydrazinyl; M is selected from a single bond, C₁-C₁₂ alkyl, a C₁-C₁₂ ester group, or a C₂-C₁₂ ether group. In the Structural formula IV, a ring formed by X, Y, and Z is a benzene ring or a heterocyclic ring, and the benzene ring and the heterocyclic ring are able to carry any one or more of C₁-C₆ alkyl, C₁-C₆ alkoxy, amino, carboxylic acid, nitro, and halogen.

In the Structural formula IV, Z is not limited to one atom or group, and a ring formed by X, Y, and Z is a benzene ring or a heterocyclic ring, the heterocyclic ring may be saturated or unsaturated, and the benzene ring and the heterocyclic ring may carry any one or more of C₁-C₆ alkyl, C₁-C₆ alkoxy, amino, carboxylic acid, nitro, and halogen, that is to say, the Structural formula IV indicates a heterocyclic compound in which the benzene ring or the heterocyclic ring is incorporated with nitrogen heterocycle. In the Structural formula III or Structural formula IV, the number of sulfydryl may be 1-6.

As an example, the compound that forms the complex with copper in the copper complex (E) may be selected from any one or more of mercaptopyrimidine, 4,6-diamino-2-mercaptopyrimidine, mercaptoimidazole, mercaptobenzimidazole, 2-mercapto-5-carboxybenzimidazole, 2-mercapto-5-nitrobenzimidazole, 5-amino-2-benzimidazolethiol, 2-mercapto-1H-benzo[d]iMidazole-4-carboxylic acid, mercaptobenzothiazole, 3-mercaptoindole, 1,3,5-tris(thioethyl)-1,3,5-triazine-2,4,6-trione, mercaptopurine, 6-thioguanine, trithiocyanuric acid, 2,6-dithiopurine, 4-thiouracil, 2-mercaptobenzoxazole, 4,6-diamino-2,6-mercaptopyrimidine, 4-thiouracil, 2-mercapto-4-hydroxy-5,6-diaminopyrimidine, 4,6-dimethyl-2-mercaptopyrimidine, dithiouracil, 2-mercaptopyrazine, 3,6-dimercaptopyridazine, 2-mercaptoimidazole, 2-mercaptothiazole, 8-mercaptoadenine, 4-thiouracil, mercaptotriazole, a 3-mercapto-1,2,4-triazole disulfide, 3-amino-5-mercapto-1,2,4-triazole, 4-methyl-1,2,4-triazole-3-thiol, 3-amino-5-mercapto-1,2,4-triazole, 3-mercapto-1,2,4-triazole, a 6,7-dihydro-6-mercapto-5H-pyrazolo[1,2-A][1,2,4]triazole chloride, 4-amino-3-hydrazino-1,2,4-triazol-5-thiol, 5-mercapto-1-methyltetrazole, 2-(5-mercaptotetrazole-1-yl)ethanol, 1-[2-(dimethylamino)ethyl]-1H-tetrazole-5-thiol, 1-phenyltetrazole-5-thiol, 1,2-dihydro-1-(4-methoxyphenyl)-5H-tetrazole-5-thione, 1-ethyl-1H-1,2,3,4-tetrazole-5-thiol, 5-mercapto-1H-tetrazole-1-acetic acid, 5-mercapto-1H-tetrazole-1-methane sulphonic acid, N-[3-(5-mercapto-1H-1,2,3,4-tetraazol-1-yl)phenyl]acetamide, 1-(4-hydroxyphenyl)-2H-tetrazole-5-thione, 1-(4-ethoxyphenyl)-5-mercapto-1H-tetrazole, 1-(4-carboxyphenyl)-5-mercapto-1H-tetrazole, and 4-amino-2-mercaptopyrimidine.

Structural formulas of part of the compounds are shown as follows. It is to be noted that, due to a rearrangement effect of electrons, the chemical structural formulas show that, the sulfur atoms and the heterocyclic ring may be connected with a single bond or a double bond and a nitrogen-containing heterocyclic ring.

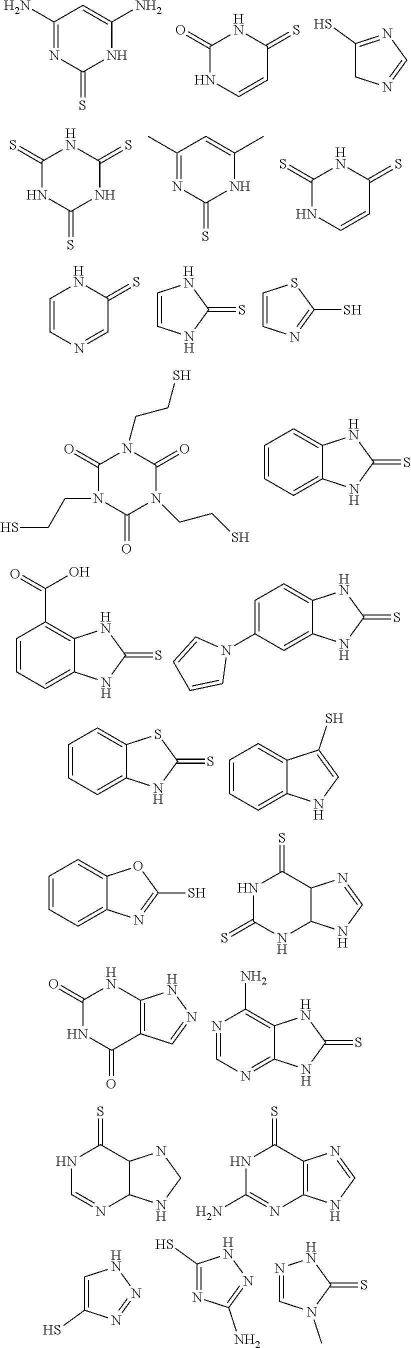

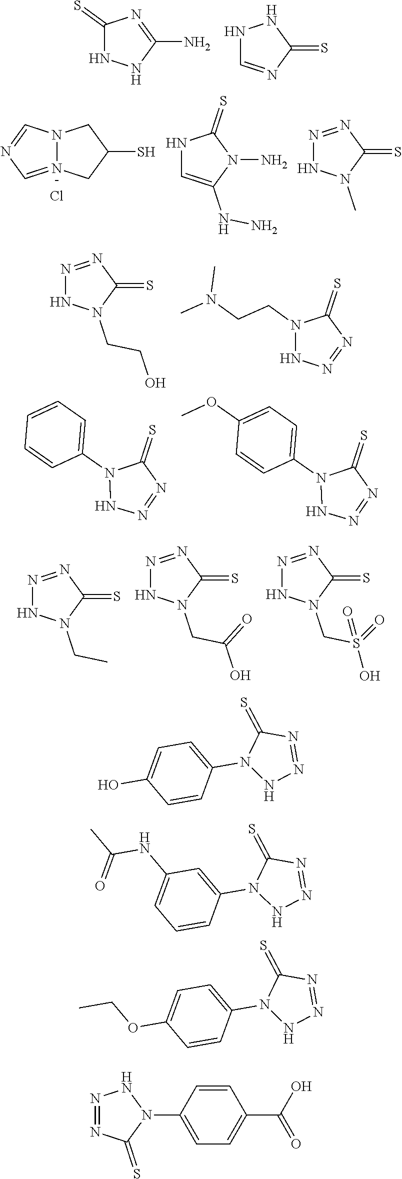

The photopolymerization monomer may be selected from the related art. In some embodiments of the present application, when the sensitizer in the dry film resist includes the compound containing the anthracene-substituted and/or triarylamine-substituted pyrazoline structure, in order to further improve the resolution ratio and adhesion of the dry film resist, the photopolymerization monomer (B) includes any one or more of the following compounds shown in structural formulas B1, B2, and B3.

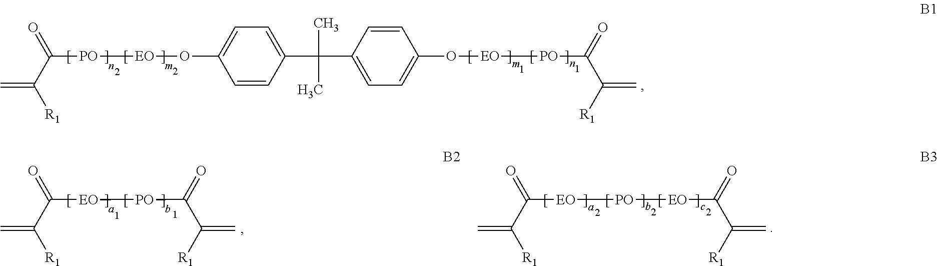

R₁s are independently H or CH₃ respectively, EO represents the oxoethylidene, PO represents the oxypropylidene, and EO and PO repeating units are arranged in a random or segmented manner; m₁ and m₂ are respectively any one of integers among 1-20, n₁ and n₂ are respectively any one of integers among 0-20, m₁+m₂ is any one of integers among 2-20, and n₁+n₂ is any one of integers among 0-20; a₁ is any one of integers among 4-20, and b₁ is any one of integers among 0-20; a₂ is any one of integers among 3-20, b₂ is any one of integers among 0-20, and c₂ is any one of integers among 3-20.

In the dry film resist system, in order to improve the resolution ratio and adhesion more evenly, a photopolymerization monomer having a structure shown in the structural formula B1 in the photopolymerization monomer (B) accounts for 40%-90% of a total amount of the photopolymerization monomer (B), further preferably accounts for 50%-80%, and is 20%-40% of a total weight of the photopolymerization monomer (B) and the alkali-soluble resin (A).

In order to further improve the adhesion of the dry film resist, in some embodiments of the present application, the photopolymerization monomer (B) may further include a multifunctional photopolymerization monomer shown in the formula B4 below. In the formula, R₁s are independently H or CH₃ respectively, and a₃s are independently any integers among 1-10 respectively.

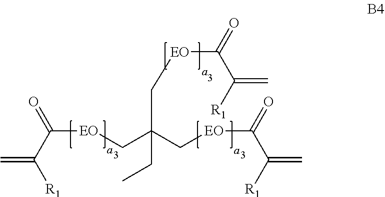

Preferably, in addition to the photopolymerization monomer, the photopolymerization monomer further contains some other common mono, bifunctional or multifunctional acrylate and/or methacrylate alkene-unsaturated double-bonded monomers. The multifunctional photopolymerization monomer may be a trifunctional photopolymerization monomer as shown in the structural formula B4, or may also be other tetrafunctional photopolymerization monomer, pentafunctional photopolymerization monomer, or hexafunctional photopolymerization monomer.

In some embodiments of the present application, the photopolymerization monomer (B) is selected from any one or more of lauryl acrylate, lauryl methacrylate, octadecyl acrylate, octadecyl methacrylate, nonylphenol acrylate, isobornyl methacrylate, tetrahydrofurfuryl acrylate, bisphenol A diacrylate, bisphenol A dimethacrylate, poly ethylene glycol diacrylate, poly propylene glycol diacrylate, poly ethylene glycol dimethacrylate, poly propylene glycol dimethacrylate, ethoxylated neopentyl glycol diacrylate, propoxylated neopentyl glycol diacrylate, trimethylolpropane triacrylate, trimethylol propane trimethacrylate, ethoxylated trimethylolpropane trimethacrylate, propoxylated trimethylolpropane trimethacrylate, ethoxylated trimethylolpropane triacrylate, propoxylated trimethylolpropane triacrylate, ethoxylated pentaerythritol triacrylate, propoxylated pentaerythritol triacrylate, ethoxylated pentaerythritol tetraacrylate, propoxylated pentaerythritol tetraacrylate, ethoxylated dipentaerythritol pentaacrylate, propoxylated dipentaerythritol pentaacrylate, propoxylated dipentaerythritol hexaacrylate and ethoxylated dipentaerythritol hexaacrylate.

For a total part by weight, the photopolymerization monomer is preferably 30-50 parts. If the part by weight is too low, low sensitivity and low resolution ratio easily occur in the photosensitive resin composition; and if the part by weight is too high, a photosensitive layer is prone to excessive glue.

When the sensitizer in the dry film resist includes the compound shown in the Structural formula I or Structural formula II, in some embodiments of the present application, in order to further improve the resolution ratio and adhesion of the dry film resist, the photopolymerization monomer (B) component contains at least any one or more of the following compounds shown in the structural formula B1, i.e., EO/PO-modified bisphenol A structure acrylate or methacrylate.

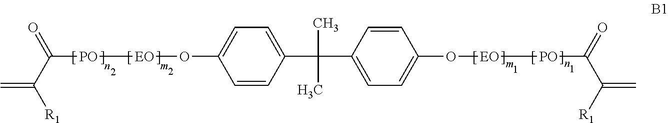

In the formula, R₁s are independently H or CH₃ respectively, m₁ and m₂ are respectively any one of integers among 1-20, n₁ and n₂ are respectively any one of integers among 0-20, m₁+m₂ is any one of integers among 2-20, n₁+n₂ is any one of integers among 0-20, EO represents the oxoethylidene, PO represents the oxypropylidene, and the EO and PO repeating units are arranged in a random or segmented manner.

In the dry film resist system, in order to improve the resolution ratio and adhesion more evenly, the photopolymerization monomer having a structure shown in the Structural formula IV accounts for 20%-80% of the total amount of the photopolymerization monomer (B), further preferably accounts for 40%-80%, and is 10%-35% of the total weight of the photopolymerization monomer (B) and the alkali-soluble resin (A). Preferably, in addition to the photopolymerization monomer, the photopolymerization monomer (B) may further contain some other common mono, bifunctional or multifunctional acrylate or methacrylate alkene-unsaturated double-bonded monomers. In some embodiments of the present application, the photopolymerization monomer (B) further includes any one or more selected from lauryl acrylate, lauryl methacrylate, octadecyl acrylate, octadecyl methacrylate, nonylphenol acrylate, isobornyl methacrylate, tetrahydrofurfuryl acrylate, bisphenol A diacrylate, bisphenol A dimethacrylate, poly ethylene glycol diacrylate, poly propylene glycol diacrylate, poly ethylene glycol dimethacrylate, poly propylene glycol dimethacrylate, ethoxylated neopentyl glycol diacrylate, propoxylated neopentyl glycol diacrylate, trimethylolpropane triacrylate, trimethylolpropane trimethacrylate, ethoxylated trimethylolpropane trimethacrylate, propoxylated trimethylolpropane trimethacrylate, ethoxylated trimethylolpropane triacrylate, propoxylated trimethylolpropane triacrylate, pentaerythritol triacrylate, pentaerythritol tetraacrylate, dipentaerythritol pentaacrylate, and dipentaerythritol hexaacrylate.

In the dry film resist system in which the sensitizer includes the compound shown in the Structural formula I or Structural formula II, for the total part by weight, the photopolymerization monomer is preferably 35-50 parts. If the part by weight is too low, poor sensitivity and poor resolution ratio easily occur in the photosensitive resin composition; and if the part by weight is too high, the photosensitive layer is prone to excessive glue.

When the dry film resist of the present application includes the copper complex, in order to better achieve the synergistic effect of each component and further improve the electroplating resistance, development capacity, and analysis capacity of the dry film resist, the photopolymerization monomer (B) includes any one or more of the following compounds shown in the structural formulas B1, B5, B6, and B7.

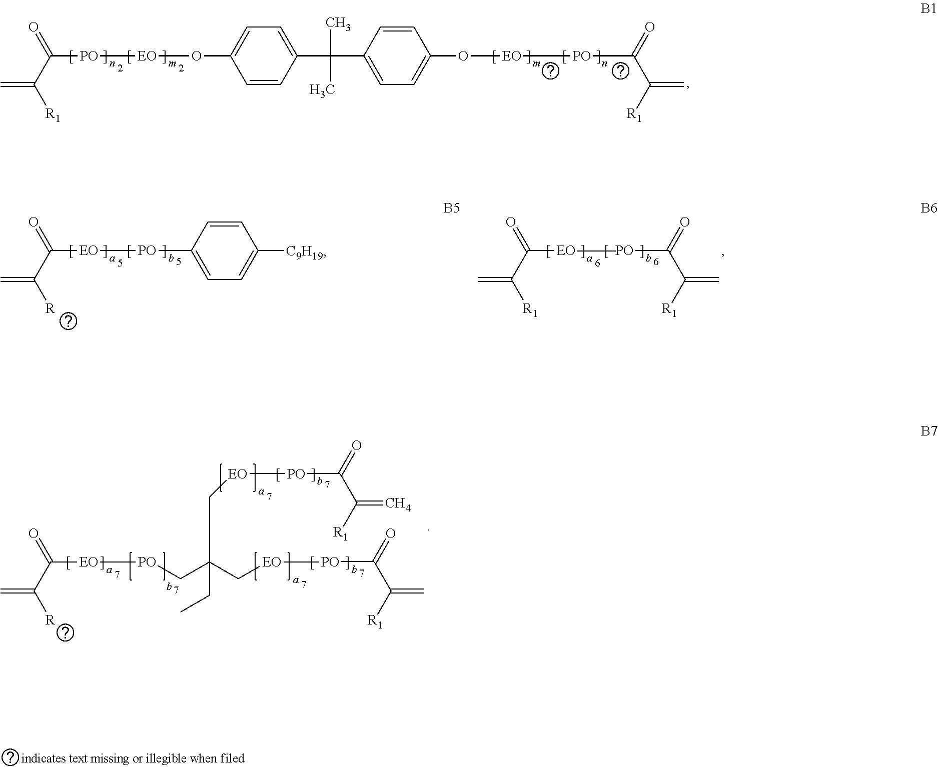

In the formula, R₁s are independently H or CH₃ respectively, and the EO chain segment and PO chain segment repeating units are arranged in a random or segmented manner; in the structural formula B1, m₁ and m₂ are respectively any one of integers among 0-30, n₁ and n₂ are respectively any one of integers among 0-20, m₁+m₂ is any one of integers among 0-30, and n₁+n₂ is any one of integers among 0-20; in the structural formula B5, a₅ is any integer among 0-30, and b₅ is any integer among 0-20; in the structural formula B6, a₆ is any integer among 0-30, b₆ is any integer among 0-20; in the structural formula B7, a₇ is any integer among 0-20, and b₇ is any integer among 0-20. Preferably, a molar quantity of the PO chain segment is 15%-60% of a total molar quantity of the EO chain segment and the PO chain segment in the photopolymerization monomer. Further preferably, the photopolymerization monomer (B) further includes any one or more selected from lauryl acrylate, lauryl methacrylate, octadecyl acrylate, octadecyl methacrylate, nonylphenol acrylate, isobornyl methacrylate, tetrahydrofurfuryl acrylate, bisphenol A dimethacrylate, bisphenol A diacrylate, poly ethylene glycol diacrylate, poly propylene glycol diacrylate, poly ethylene glycol dimethacrylate, poly propylene glycol dimethacrylate, ethoxylated neopentyl glycol diacrylate, propoxylated neopentyl glycol diacrylate, trimethylolpropane triacrylate, trimethylolpropane trimethacrylate, ethoxylated trimethylolpropane trimethacrylate, propoxylated trimethylolpropane trimethacrylate, ethoxylated trimethylolpropane triacrylate, propoxylated trimethylolpropane triacrylate, pentaerythritol triacrylate, pentaerythritol tetraacrylate, dipentaerythritol pentaacrylate, dipentaerythritol hexaacrylate, and polyurethaneacrylate. The alkali-soluble resin (A) of the present application may be selected from the existing technologies, in some embodiments of the present application, in order to improve the comprehensive performance of the dry film resist, the alkali-soluble resin (A) includes any one or more of the following structures shown in a structural formula A1.

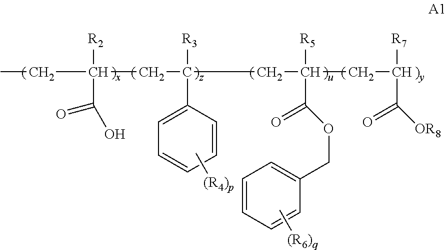

R₂, R₃, R₅, and R₇ are independently hydrogen or methyl respectively; R₄ and R₆ are independently alkyl having 1-5 carbon atoms, alkoxy having 1-5 carbon atoms, hydroxyl or halogen atoms; p and q independently represent any integer among 0-5 respectively; R₈ is any one of straight chain, branched chain or cyclic alkyl having 1-18 carbon atoms; and x, y, z, and u respectively represent specific weights of various copolymerization components in the alkali-soluble resin, wherein x is 15-40 wt %, z is 0-50 wt %, u is 0-80 wt %, and y is 0-40 wt %.

In some embodiments of the present application, an acid value of the alkali-soluble resin (A) is 120-250 mg KOH/g, this is because when the acid value of the alkali-soluble resin is too small, there is a tendency for alkali solubility to deteriorate, and the development and film removal time to become longer; and when the acid value of the alkali-soluble resin is too large, there is a tendency for the resolution ratio to worsen.

Preferably, in order to improve the comprehensive performance of the dry film resist, the weight-average molecular weight of the alkali-soluble resin (A) is 30000-120000, and the molecular weight distribution is 1.3-2.5. The alkali-soluble resin (A) may be a copolymer resin shown in the structural formula A1, or may also be an alkali-soluble copolymer resin that is formed by compounding more than two copolymer resins with different molecular weights, different acid values or different styrene contents, and preferably, a polymerization conversion rate is greater than or equal to 97%.

In some embodiments of the present application, when the sensitizer in the dry film resist includes the compound containing the anthracene-substituted and/or triarylamine-substituted pyrazoline structure, in order to better achieve the synergistic effect, in the structural formula A1 representing the alkali-soluble resin, x is 15-35 wt %, z is 0-50 wt %, u is 0-80 wt %, y is 0-25 wt %, and a value of z+u is 40 wt %-80 wt %, such that the photosensitivity, resolution ratio, and sensitivity of the dry film resist and the stability of the resist patterns are further improved. When the dry film resist system is used, a copolymerization unit of the alkali-soluble resin (A) is preferably selected from one or more of methyl methacrylate, methyl acrylate, ethyl methacrylate, ethyl acrylate, propyl methacrylate, propyl acrylate, isopropyl methacrylate, isopropyl acrylate, butyl methacrylate, butyl acrylate, isobutyl methacrylate, isobutyl acrylate, methylheptyl methacrylate, methylheptyl acrylate, dodecyl methacrylate, dodecyl acrylate, stearyl methacrylate, stearyl acrylate, 2-hydroxyethyl methacrylate, 2-hydroxyethyl acrylate, 2-hydroxypropyl methacrylate, 2-hydroxypropyl acrylate, 4-hydroxybutyl methacrylate, 4-hydroxybutyl acrylate, glycidyl methacrylate, glycidyl acrylate, N,N-dimethyl ethyl acrylate, N,N-dimethylethyl methacrylate, N,N-diethylethyl acrylate, N,N-diethylethyl methacrylate, N,N-diethylpropyl acrylate, N,N-diethylpropyl methacrylate, N,N-dimethylbutyl acrylate, N,N-dimethylbutyl methacrylate, N,N-diethylbutyl methacrylate and N,N-diethylbutyl acrylate. Preferably, the weight-average molecular weight of the alkali-soluble resin is 30000-80000, and the molecular weight distribution is 1.3-2.5. A narrow molecular weight distribution facilitates the improvement of the resolution ratio of the dry film resist.

When the sensitizer in the dry film resist includes the compound shown in the Structural formula I or Structural formula II, in some embodiments of the present application, in order to improve the comprehensive performance of the dry film resist, in the structural formula A1 representing the alkali-soluble resin, R₃ is hydrogen, x is 15-40 wt %, z is 0-40 wt %, u is 0, and y is 20-60 wt %. When the dry film resist system is used, a copolymerization unit of the alkali-soluble resin (A) preferably contains alkyl acrylate and/or alkyl methacrylate, for example, containing alkyl acrylate and/or alkyl methacrylate and styrene and/or derivatives thereof; the alkyl methacrylate is selected from any one or more of methyl methacrylate, ethyl methacrylate, propyl methacrylate, isopropyl methacrylate, butyl methacrylate, isobutyl methacrylate, methylheptyl methacrylate, dodecyl methacrylate, stearyl methacrylate, 2-hydroxyethyl methacrylate, 2-hydroxypropyl methacrylate, 4-hydroxybutyl methacrylate, glycidyl methacrylate, N,N-dimethylethyl methacrylate, N,N-diethylethyl methacrylate, N,N-diethylpropyl methacrylate, N,N-dimethylbutyl methacrylate, and N,N-diethylbutyl methacrylate; the alkyl acrylate is selected from any one or more of methyl acrylate, ethyl acrylate, propyl acrylate, isopropyl acrylate, butyl acrylate, isobutyl acrylate, methylheptyl acrylate, dodecyl acrylate, stearyl acrylate, 2-hydroxyethyl acrylate, 2-hydroxypropyl acrylate, 4-hydroxybutyl acrylate, glycidyl acrylate, N,N-dimethylethyl acrylate, N,N-diethylethyl acrylate, N,N-diethylpropyl acrylate, N,N-dimethylbutyl acrylate, and N,N-diethylbutyl acrylate. The copolymerization unit of the alkali-soluble resin (A) contains styrene or derivatives thereof, preferably the styrene derivatives are selected from one or more of a-methylstyrene, benzyl acrylate and benzyl methacrylate. When the benzyl acrylate and/or benzyl methacrylate is used as the copolymerization unit, the styrene copolymerization unit may not be used. It is to be noted that, compared with the styrene derivatives, the effect is better by using the styrene as a comonomer. In some preferred embodiments of the present application, the content of the styrene in the copolymerization unit of the alkali-soluble resin is 0-40 wt % of a total weight of the copolymerization unit. Preferably, the weight-average molecular weight of the alkali-soluble resin is 30000-80000, and the molecular weight distribution is 1.3-2.5. A narrow molecular weight distribution facilitates the improvement of the resolution ratio of the dry film resist.

When the dry film resist of the present application contains the copper complex, in order to further achieve the synergistic effect of each component, in the structural formula A1 representing the alkali-soluble resin (A), R₃ is hydrogen, x is 15-40 wt %, z is 0-40 wt %, u is 0, and y is 20-70 wt %, i.e., the alkali-soluble resin (A) is formed by copolymerizing acrylic acid and/or methacrylic acid, acrylate and/or methacrylate, and styrene or derivatives of the styrene. Preferably, the acrylate and/or methacrylate may be alkyl acrylate and/or alkyl methacrylate, for example, any one or more of methyl methacrylate, ethyl methacrylate, propyl methacrylate, isopropyl methacrylate, butyl methacrylate, isobutyl methacrylate, methylheptyl methacrylate, dodecyl methacrylate, stearyl methacrylate, 2-hydroxyethyl methacrylate, 2-hydroxypropyl methacrylate, 4-hydroxybutyl methacrylate, glycidyl methacrylate, N,N-dimethylethyl methacrylate, N,N-diethylethyl methacrylate, N,N-diethylpropyl methacrylate, N,N-dimethylbutyl methacrylate, and N,N-diethylbutyl methacrylate, methyl acrylate, ethyl acrylate, propyl acrylate, isopropyl acrylate, butyl acrylate, isobutyl acrylate, methylheptyl acrylate, dodecyl acrylate, stearyl acrylate, 2-hydroxyethyl acrylate, 2-hydroxypropyl acrylate, 4-hydroxybutyl acrylate, glycidyl acrylate, N,N-dimethylethyl acrylate, N,N-diethylethyl acrylate, N,N-diethylpropyl acrylate, N,N-dimethylbutyl acrylate, and N,N-diethylbutyl acrylate. The weight-average molecular weight of the alkali-soluble resin is 50000-120000, and the molecular weight distribution is 1.3-2.5. A narrow molecular weight distribution facilitates the improvement of the resolution ratio of the dry film resist.

In order to further improve the resolution ratio and the adhesion, and improve the performance of the dry film resist such as pattern shapes, from the viewpoint of inhibiting polymerization in an unexposed portion during resist pattern formation, an additive component of the dry film resist may also contain a free radical polymerization inhibitor. From the viewpoint of a production process, the additive of the present application may further include one or more of a color former, a coloring agent, a plasticizer, a photothermal stabilizer, an adhesion promoter, a leveling agent, and a defoaming agent, and may be composed according to proportions in the related art. Preferably, the content of the additive is, by mass, 0.5-5.0 parts.

The free radical polymerization inhibitor may be selected from the existing technologies, for example, selected from one or more of 4-methoxyphenol, 4-ethyl-6-tert-butylphenol, nitroso phenylhydroxylamine aluminum salt, dimethyl-1,2-benzenediol, 3-methylcatechol, 4-methylcatechol, catechol, 2-ethylcatechol, 3-ethylcatechol, 4-ethylcatechol, 2-propylcatechol, 3-propylcatechol, 4-propylcatechol, 2-n-butylcatechol, 3-n-butylcatechol, 4-n-butylcatechol, 2-tert-butylcatechol, 3-tert-butylpyrocatechol, 4-tert-butylcatechol, 3,5-di-tert-butylcatechol, resorcinol, 2-methylresorcinol, 4-methylresorcinol, 5-orcinol, 2-ethylresorcinol, 4-ethylresorcinol, 2-propylresorcinol, 4-propylresorcinol, 2-n-butylresorcinol, 4-butylresorcinol, 2-tert-butylresorcinol, 4-tert-butylresorcinol, 1,4-hydroquinone, methylhydroquinone, ethylhydroquinone, propyl hydroquinone, tert-butylhydroquinone, 2,5-di-tert-butylhydroquinone, 2,6-di-tert-butyl-4-methylphenol, pyrogallol, 4-hydroxy-2,2,6,6-tetramethylpiperidine-N-oxyl, and 2,2-methylenebis(4-methyl-6-tert-butylphenol). In some embodiments of the present application, the content of the free radical polymerization inhibitor is 0.001 wt %-0.005 wt % of the total weight of the dry film resist; when an addition is too low, the resolution ratio of the obtained dry film resist is insufficient; and when the addition is too high, the photosensitivity of the dry film resist is poor.

Another typical implementation of the present application provides a photosensitive dry film.

The photosensitive dry film includes a dry film resist layer, and a support layer and a protective layer located on two sides of the dry film resist layer. The dry film resist layer contains any one of the dry film resists described above.

Since the photosensitive dry film of the present application uses the high photosensitivity and high resolution ratio dry film resist with more stable performance in terms of sensitivity and resist patterns, the photosensitive dry film is excellent in performance in terms of photosensitive, resolution ratio, and stability, such that the photosensitive dry film is suitable for manufacturing high-order PCBs such as carrier boards and SLPs, a production yield can be increased, and production efficiency during PCB manufacturing, lead frame manufacturing, semiconductor manufacturing, and manufacturing of components such as ITOs in the field of flat panel displays is significantly improved, thereby reducing production cost.

Since the photosensitive dry film of the present application uses a direct depiction exposure imaging dry film resist that has high photosensitivity and high resolution ratio, and has high para-position recognition adaptability to different types of LDI exposure machines, the photosensitive dry film has good photosensitivity, resolution ratio, and para-position precision, such that the photosensitive dry film may be applied to different types of LDI exposure machines, so as to achieve excellent comprehensive performance such as the para-position recognition adaptability, thereby improving the production yield, significantly improving the production efficiency during PCB manufacturing, lead frame manufacturing, semiconductor manufacturing, and manufacturing of components such as ITOs in the field of flat panel displays, and thus reducing the production cost.

A typical implementation of the present application provides a dry film resist. The dry film resist includes an alkali-soluble resin (A), a photopolymerization monomer (B), a photoinitiator (C), and a copper complex (E). A compound that forms a complex with copper in the copper complex includes a nitrogen-containing heterocycle compound, the photopolymerization monomer contains an EO chain segment and a PO chain segment, the EO chain segment represents oxoethylidene, and the PO chain segment represents oxypropylidene. The EO chain segment and the PO chain segment may be from one or more photopolymerization monomers simultaneously containing the EO chain segment and the PO chain segment, or the photopolymerization monomers respectively containing the EO chain segment and the PO chain segment may also be compounded, or the photopolymerization monomers containing different ratios of the EO chain segment and the PO chain segment are compounded.

The dry film resist contains the nitrogen-containing heterocycle compound and the copper-formed complex, on the one hand, due to large polarity of a nitrogen heterocyclic structure, the copper complex can improve an acting force with copper, and on the other hand, lone pair electrons on nitrogen atoms can coordinate and complex with copper atoms, and a binding force between the compound and the copper atoms can be further improved through the generation of such coordination chemical bonds. Through the effects of the above two aspects, the binding force between the dry film resist and a copper surface can be significantly improved by adding the copper complex, such that a phenomenon that a dry film slightly warps on a bottom side wall during a plating process is improved, thereby improving the electroplating resistance of the dry film resist.

The photopolymerization monomer in the dry film resist simultaneously contains a hydrophilic group Ethylene Oxide (EO) and a hydrophobic group Propylene Oxide (PO). On the one hand, the photopolymerization monomer containing a hydrophilic group EO chain segment may improve the water-solubility of the dry film resist, such that the development performance and resolution ratio of the dry film resist are improved; and on the other hand, when the photopolymerization monomer only contains the EO chain segment, the water-solubility of the dry film resist is improved, the dry film resist is susceptible to dry film sidewall wrapping due to swelling, causing an undesirable phenomenon of cementation, in the photopolymerization monomer, by adding a hydrophobic group PO chain segment, the hydrophobicity of the dry film resist may be improved to a certain extent, thereby improving the electroplating resistance of the dry film resist. Therefore, the photopolymerization monomer chemically balances the hydrophilicity and hydrophobicity of the dry film resist, and a dry film resist photosensitive material with balanced development performance, analysis capacity, and electroplating resistance can be obtained.

Specific selection of the alkali-soluble resin (A), the photopolymerization monomer (B), the photoinitiator (C), and the copper complex (E) in the dry film resist may refer to the foregoing content of the present application. In some preferred embodiments of the present application, by weight, the dry film resist includes 40-65 parts of the alkali-soluble resin (A), 35-60 parts of the photopolymerization monomer (B), 2.0-4.5 parts of the photoinitiator (C), and 0.01-0.5 parts of the copper complex (E), such that the dry film resist has better comprehensive performance. Preferably, the dry film resist may further include 0.01-0.5 parts of a sensitizer (D). Preferably, the sensitizer (D) has any one of the following structures shown in a Structural formula I or Structural formula II.

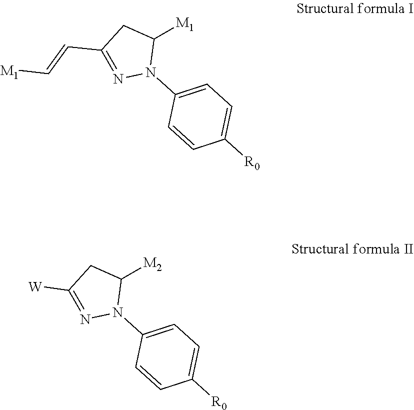

R₀s are independently hydrogen, halogen, C₁-C₈ alkyl, and C₁-C₄ alkoxy, respectively; modified groups M₁s in the Structural formula I are independently biphenyl, a condensed ring group or electron-rich heterocycle, fused electron-rich heterocycle, or a benzene ring with C₁-C₄ alkoxy or amino; a modified group M₂ in the Structural formula II is selected from any one of biphenyl, a condensed ring group or electron-rich heterocycle, fused electron-rich heterocycle, or a benzene ring with C₁-C₄ alkoxy or amino, or phenyl with C₁-C₄ alkoxy substituents; and a modified group W is any one of a benzene ring and a fluorene ring, or is a benzene ring, a biphenyl ring, or a fluorene ring with any one or more of halogen, C₁-C₈ alkyl, and C₁-C₄ alkoxy substituents. The biphenyl groups in the modified groups M₁ and M₂ represent biphenyl or derivatives thereof; the corresponding condensed ring group and electron-rich heterocycle or fused electron-rich heterocycle represent the corresponding groups or derivatives thereof; and types of the derivatives of the above groups may be conventional types in the art, for example, containing substituents such as halogen, C₁-C₈ alkyl, C₁-C₄ alkoxy, or the like.

In the sensitizer, some benzene rings and condensed ring groups of electron-donating substituents, or modified groups with electron-rich heterocyclic or fused-heterocyclic compounds are introduced into the pyrazoline compound, such that an entire molecular structure is an electron-rich conjugated system, such electron-rich conjugation effect facilitates the promotion of red shift of an absorption spectrum of the sensitizer, and the absorption spectrum may extend to a visible region. The absorption wavelength is closer to an exposure light source with a wavelength being 405 nm, which is more sensitive to the exposure light source, such that the photosensitivity of the dry film resist to an LDI 405 nm exposure light source is improved.

Preferably, the modified groups M₁s are independently condensed ring groups with any one or more of C₁-C₄ alkoxy, amino, and alkyl respectively, or are any one of furan, thiophene, indole, thiazole, benzofuran, benzothiazole, indene, anthracene, acridine, and aromatic amine. Further preferably, a specific structural formula of the modified group M₁ and/or M₂ includes:

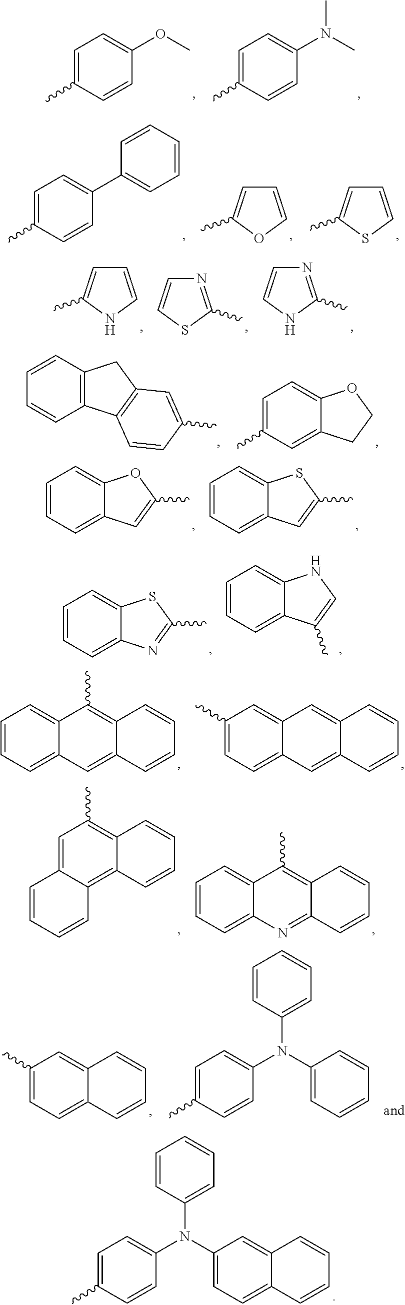

[symbol]

is a binding position of a group. Optionally, the benzene ring, biphenyl ring, fused ring, or heterocyclic ring in the specific structural formula of the modified group M₁ and/or M₂ contains any one or more of substituents in halogen, C₁-C₅ alkyl, and C₁-C₄ alkoxy, and preferably, the substituent is located in a para-position.

In addition, from extensive research and experimentation, the applicants found that, when the sensitizer is used, and a weight ratio of the sensitizer to the copper complex is within a range of 30:1-1:50, the comprehensive performance of the dry film resist, especially electroplating resistance, is good, for example, the weight ratio of the sensitizer to the copper complex is 20:1, 10:1, 5:1, 2:1, 1;1, 1:5, 1:10, or 1:20.

Still another typical implementation of the present application provides a photosensitive dry film. The photosensitive dry film includes a dry film resist layer, and a support layer and a protective layer located on two sides of the dry film resist layer. The dry film resist layer contains any one of the dry film resists described above. As the dry film resist contains the nitrogen-containing heterocycle compound and the copper-formed complex, on the one hand, due to large polarity of a nitrogen heterocyclic structure, the copper complex can improve an acting force with copper, and on the other hand, lone pair electrons on nitrogen atoms can coordinate and complex with copper atoms, and a binding force between the compound and the copper atoms can be further improved through the generation of such coordination chemical bonds. Through the effects of the above two aspects, the binding force between the dry film resist and a copper surface can be significantly improved by adding the copper complex, such that a phenomenon that a dry film slightly warps on a bottom side wall during a plating process is improved, thereby improving the electroplating resistance of the dry film resist.

Yet another typical implementation of the present application provides a copper clad laminate. The copper clad laminate is provided with any one of the dry film resists described above. Since the copper clad laminate of the present application is provided with the dry film resist having good electroplating resistance, the copper clad laminate has good electroplating resistance, such that a yield of products during manufacturing can be increased.

The beneficial effects that the present application may be achieved are further described below with reference to the embodiments and comparative examples.

### Synthesis Methods of Sensitizer Compounds is as Follows

### Synthesis of sensitizer compound D-1

Raw materials of 9-anthraldehyde (62 g), acetone (7 g), and ethanol (150 mL) were added in a 500 mL three-necked flask, the flask was placed in a water bath at room temperature, stirring was performed for 15 min, after the raw materials were dissolved, a 10% NaOH aqueous solution (120 g) was added dropwise to the flask, adding time was 1 h, after the adding was completed, stirring was continuously performed at room temperature to react for 8h, a reaction was monitored through a point TLC plate, and after the reaction had ceased to change, the reaction was stopped. Filtration under a reduced pressure was performed on a suspended solid obtained in the reaction, an obtained solid crude product was dispersed in small amount of ethanol (100 mL), after 30 min of stirring at room temperature, suction filtration was performed to collect a solid, drying was performed by using a rotary evaporator, small amount of a solvent encapsulated in the solid was removed, and then an intermediate product compound 1 (49 g, purity 91%) as shown in the following Reaction equation I was obtained.

The intermediate product compound 1 (49 g) and glacial acetic acid (150 g) were added in a 500 mL three-necked flask, the flask was placed in an oil bath for stirring, a temperature was risen to 50° C., phenylhydrazine (22 g) was slow added dropwise at 50° C., the adding time lasted for 1h, and after adding was completed, the temperature was risen to 80° C. for reaction for 8 h. The reaction was monitored through the point TLC plate, the raw materials were completely consumed, then the reaction was stopped; after the temperature was cooled to room temperature, the ethanol (200 mL) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-1 (44 g, purity 95%) as shown in the following reaction equation was obtained.

A Specific Reaction Reaction Equation I was Shown as Follows.

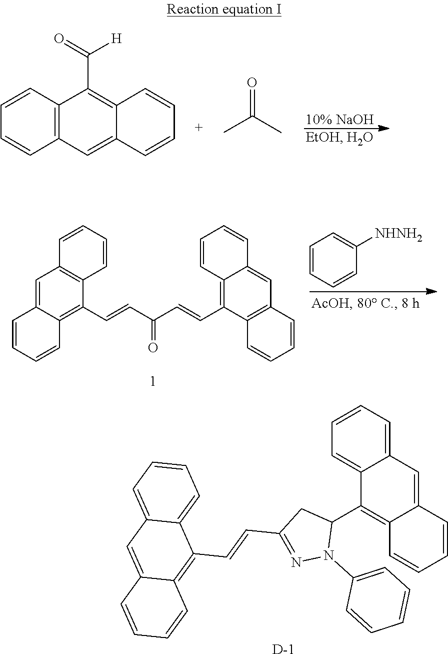

A sensitizer compound structure in the reaction equation above was only used as an example, an anthracene ring might carry substituents such as halogen, alkyl with a carbon chain length being C₁-C₈, and alkoxy with a carbon chain length being C₁-C₄, and the number of substituents might be one, or 1-5.

### Synthesis of Sensitizer Compound D-2

Raw materials of 2-acetylfluorene (21.8 g), 9-anthraldehyde (20.6 g), and ethanol (100 mL) were added in a 250 mL three-necked flask, the flask was placed in a water bath at room temperature, stirring was performed for 15 min, after the raw materials were dissolved, a 3 mol/L NaOH aqueous solution (66 mL) was added dropwise to the flask, adding time was 1h, after the adding was completed, stirring was continuously performed at room temperature to react for 8h, a reaction was monitored through a point TLC plate, and after the raw material was completely consumed, the reaction was stopped. Filtration under a reduced pressure was performed on a suspended solid obtained in the reaction, an obtained solid crude product was dispersed in small amount of ethanol (100 mL), after 30 min of stirring at room temperature, suction filtration was performed to collect a solid, drying was performed by using a rotary evaporator, small amount of a solvent encapsulated in the solid was removed, and then an intermediate product compound 2 (36 g, purity 90%) as shown in the following reaction equation was obtained.

The intermediate product compound 2 (36 g) and glacial acetic acid (100 g) were added in a 250 mL three-necked flask, the flask was placed in an oil bath for stirring, a temperature was risen to 50° C., phenylhydrazine (19 g) was slow added dropwise at 50° C., the adding time lasted for 0.5h, and after adding was completed, the temperature was risen to 80° C. for reaction for 8 h. The reaction was monitored through the point TLC plate, the raw materials were completely consumed, then the reaction was stopped; after the temperature was cooled to room temperature, the ethanol (150 mL) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-2 (28 g, purity 96%) as shown in the following reaction equation was obtained.

A Specific Reaction Equation II was Shown as Follows.

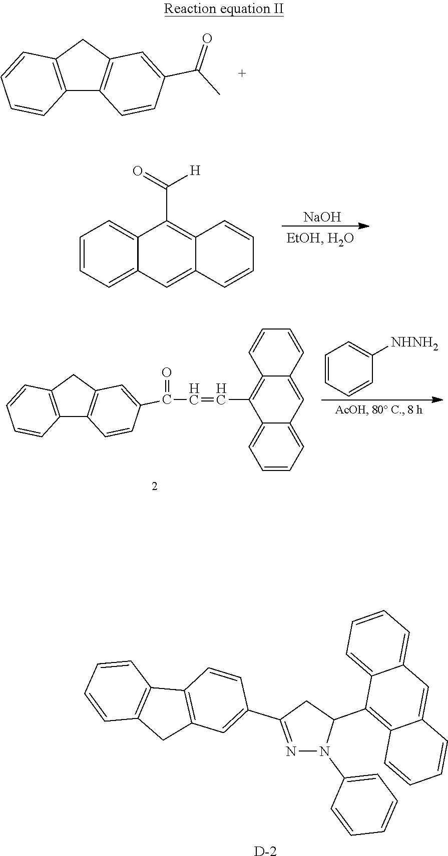

A sensitizer compound structure in the formula above was only used as an example, an anthracene ring and a fluorene ring might carry substituents such as halogen, alkyl with a carbon chain length being C₁-C₈, and alkoxy with a carbon chain length being C₁-C₄, and the number of substituents might be one, or 1-5.

### Synthesis of Sensitizer Compound D-3

Raw materials of acetophenone (12.6 g), 9-anthraldehyde (20.6 g), and ethanol (100 mL) were added in a 250 mL three-necked flask, the flask was placed in a water bath at room temperature, stirring was performed for 15 min, after the raw materials were dissolved, a 3 mol/L NaOH aqueous solution (66 mL) was added dropwise to the flask, adding time was 1h, after the adding was completed, stirring was continuously performed at room temperature to react for 8h, a reaction was monitored through a point TLC plate, and after the raw material was completely consumed, the reaction was stopped. Filtration under a reduced pressure was performed on a suspended solid obtained in the reaction, an obtained solid crude product was dispersed in small amount of ethanol (100 mL), after 30 min of stirring at room temperature, suction filtration was performed to collect a solid, drying was performed by using a rotary evaporator, small amount of a solvent encapsulated in the solid was removed, and then an intermediate product compound 3 (24 g, purity 91%) as shown in the following reaction equation was obtained.

The intermediate product compound 3 (24 g) and glacial acetic acid (100 g) were added in a 250 mL three-necked flask, the flask was placed in an oil bath for stirring, a temperature was risen to 50° C., phenylhydrazine (15 g) was slow added dropwise at 50° C., the adding time lasted for 20 min, and after adding was completed, the temperature was risen to 80° C. for reaction for 8 h. The reaction was monitored through the point TLC plate, the raw materials were completely consumed, then the reaction was stopped; after the temperature was cooled to room temperature, the ethanol (150 mL) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-3 (19 g, purity 97%) as shown in the following reaction equation was obtained.

A Specific Reaction Equation III was Shown as Follows.

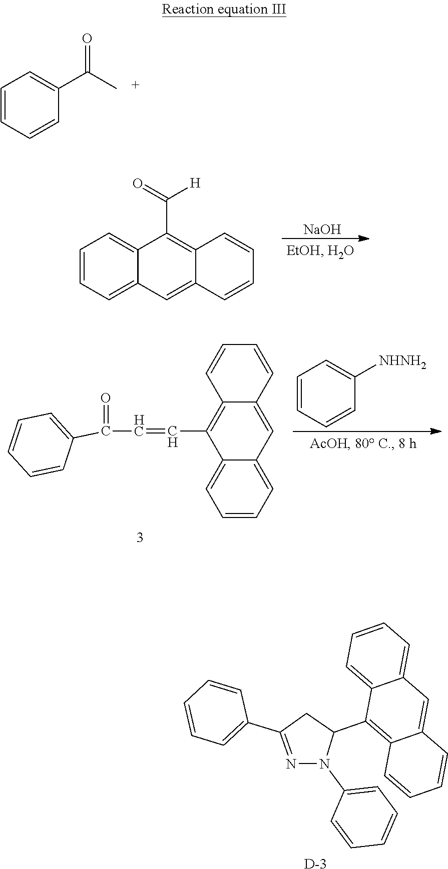

A sensitizer compound structure in the formula above was only used as an example, a benzene ring and an anthracene ring might carry substituents such as halogen, alkyl with a carbon chain length being C₁-C₈, and alkoxy with a carbon chain length being C₁-C₄, and the number of substituents might be one, or 1-5.

### Synthesis of Sensitizer Compound D-4

Raw materials of 4-acetylbiphenyl (20.5 g), 9-anthraldehyde (20.6 g), and ethanol (100 mL) were added in a 250 mL three-necked flask, the flask was placed in a water bath at room temperature, stirring was performed for 15 min, after the raw materials were dissolved, a 3 mol/L NaOH aqueous solution (66 mL) was added dropwise to the flask, adding time was 1h, after the adding was completed, stirring was continuously performed at room temperature to react for 8h, a reaction was monitored through a point TLC plate, and after the raw material was completely consumed, the reaction was stopped. Filtration under a reduced pressure was performed on a suspended solid obtained in the reaction, an obtained solid crude product was dispersed in small amount of ethanol (100 mL), after 30 min of stirring at room temperature, suction filtration was performed to collect a solid, drying was performed by using a rotary evaporator, small amount of a solvent encapsulated in the solid was removed, and then an intermediate product compound 4 (32 g, purity 92%) as shown in the following reaction equation was obtained.

The intermediate product compound 4 (32 g) and glacial acetic acid (100 g) were added in a 250 mL three-necked flask, the flask was placed in an oil bath for stirring, a temperature was risen to 50° C., phenylhydrazine (17 g) was slow added dropwise at 50° C., the adding time lasted for 20 min, and after adding was completed, the temperature was risen to 80° C. for reaction for 8 h. The reaction was monitored through the point TLC plate, the raw materials were completely consumed, then the reaction was stopped; after the temperature was cooled to room temperature, the ethanol (150 mL) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-4 (26 g, purity 96%) as shown in the following reaction equation was obtained.

A Specific Reaction Equation IV was Shown as Follows.

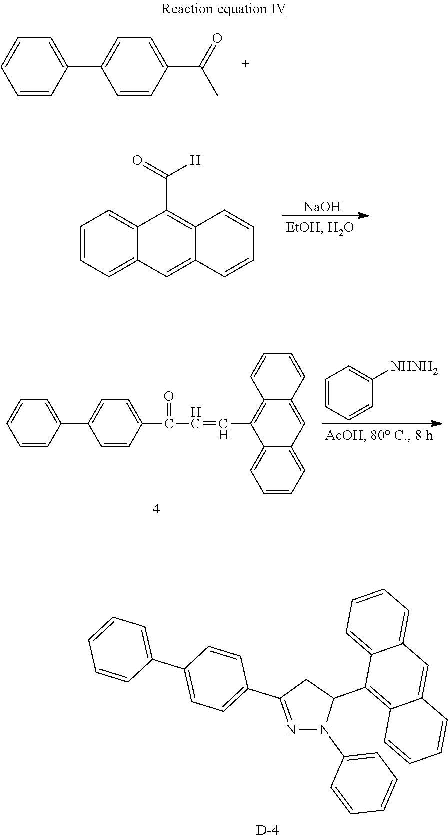

A sensitizer compound structure in the formula above was only used as an example, a biphenyl ring and an anthracene ring might carry substituents such as halogen, alkyl with a carbon chain length being C₁-C₈, and alkoxy with a carbon chain length being C₁-C₄, and the number of substituents might be one, or 1-5.

### Synthesis of Sensitizer Compound D-5

Raw materials of 2-acetonaphthone (17.8 g), 4-(N,N-diphenylamino)benzaldehyde (27.3 g), and ethanol (100 mL) were added in a 250 mL three-necked flask, the flask was placed in a water bath at room temperature, stirring was performed for 15 min, after the raw materials were dissolved, a 3 mol/L NaOH aqueous solution (66 mL) was added dropwise to the flask, adding time was 1h, after the adding was completed, stirring was continuously performed at room temperature to react for 8h, a reaction was monitored through a point TLC plate, and after the raw material was completely consumed, the reaction was stopped. Filtration under a reduced pressure was performed on a suspended solid obtained in the reaction, an obtained solid crude product was dispersed in small amount of ethanol (100 mL), stirring was performed for 30 min at room temperature, and an intermediate product compound 5 (30 g, purity 91%) as shown in the following reaction equation was obtained.

The intermediate product compound 5 (30 g) and glacial acetic acid (100 g) were added in a 250 mL three-necked flask, the flask was placed in an oil bath for stirring, a temperature was risen to 50° C., phenylhydrazine (14.7 g) was slow added dropwise at 50° C., the adding time lasted for 20 min, and after adding was completed, the temperature was risen to 80° C. for reaction for 8 h. The reaction was monitored through the point TLC plate, the raw materials were completely consumed, then the reaction was stopped; after the temperature was cooled to room temperature, the ethanol (150 mL) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-5 (33 g, purity 95%) as shown in the following reaction equation was obtained.

A Specific Reaction Equation V was Shown as Follows.

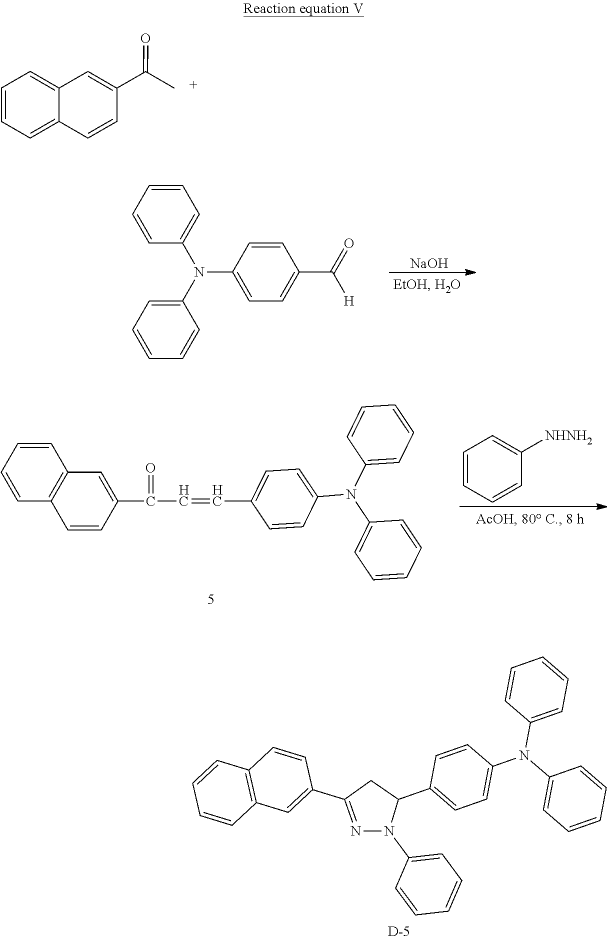

A sensitizer compound structure in the formula above was only used as an example, a naphthalene ring and a benzene ring might carry substituents such as halogen, alkyl with a carbon chain length being C₁-C₈, and alkoxy with a carbon chain length being C₁-C₄, and the number of substituents might be one, or 1-5.

### Synthesis of Sensitizer Compound D-6

Raw materials of 3-acetylthiophene (13.2 g), 4-(N,N-diphenylamino)benzaldehyde (27.3 g), and ethanol (100 mL) were added in a 250 mL three-necked flask, the flask was placed in a water bath at room temperature, stirring was performed for 15 min, after the raw materials were dissolved, a 3 mol/L NaOH aqueous solution (66 mL) was added dropwise to the flask, adding time was 1h, after the adding was completed, stirring was continuously performed at room temperature to react for 8h, a reaction was monitored through a point TLC plate, and after the raw material was completely consumed, the reaction was stopped. Filtration under a reduced pressure was performed on a suspended solid obtained in the reaction, an obtained solid crude product was dispersed in small amount of ethanol (100 mL), after 30 min of stirring at room temperature, suction filtration was performed to collect a solid, drying was performed by using a rotary evaporator, small amount of a solvent encapsulated in the solid was removed, and then an intermediate product compound 6 (26 g, purity 90%) as shown in the following reaction equation was obtained.

The intermediate product compound 6 (26 g) and glacial acetic acid (100 g) were added in a 250 mL three-necked flask, the flask was placed in an oil bath for stirring, a temperature was risen to 50° C., phenylhydrazine (14 g) was slow added dropwise at 50° C., the adding time lasted for 20 min, and after adding was completed, the temperature was risen to 80° C. for reaction for 8 h. The reaction was monitored through the point TLC plate, the raw materials were completely consumed, then the reaction was stopped; after the temperature was cooled to room temperature, the ethanol (150 mL) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-6 (21 g, purity 96%) as shown in the following reaction equation was obtained.

A Specific Reaction Equation VI was Shown as Follows.

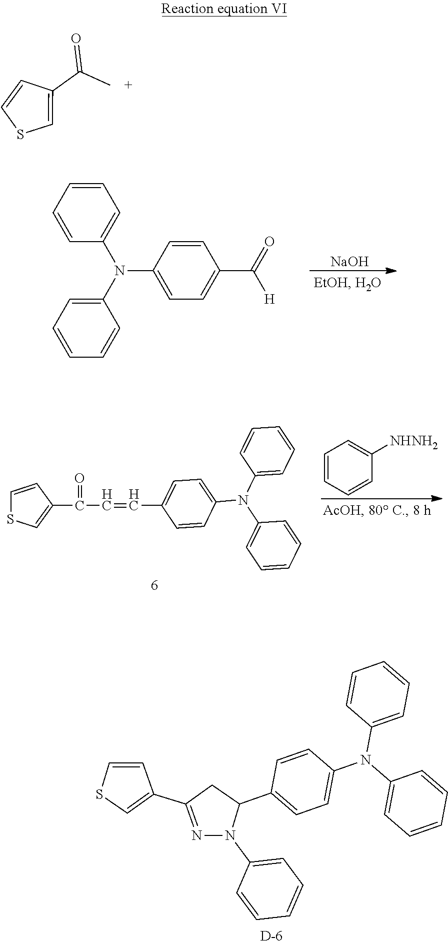

A sensitizer compound structure in the formula above was only used as an example, a benzene ring and a heterocyclic ring might carry substituents such as halogen, alkyl with a carbon chain length being C₁-C₈, and alkoxy with a carbon chain length being C₁-C₄, and the number of substituents might be one, or 1-5.

### Synthesis of Sensitizer Compound D-7

Raw materials of 4-(N,N-diphenylamino)benzaldehyde (49.6 g), acetone (5.8 g), and ethanol (150 mL) were added in a 500 mL three-necked flask, the flask was placed in a water bath at room temperature, stirring was performed for 15 min, after the raw materials were dissolved, a 10% NaOH aqueous solution (160 g) was added dropwise to the flask, adding time was 1h, after the adding was completed, stirring was continuously performed at room temperature to react for 8h, a reaction was monitored through a point TLC plate, and after the reaction had ceased to change, the reaction was stopped. Filtration under a reduced pressure was performed on a suspended solid obtained in the reaction, an obtained solid crude product was dispersed in small amount of ethanol (150 mL), after 30 min of stirring at room temperature, suction filtration was performed to collect a solid, drying was performed by using a rotary evaporator, small amount of a solvent encapsulated in the solid was removed, and then an intermediate product compound 7 (41 g, purity 91%) as shown in the following reaction equation was obtained.

The intermediate product compound 7 (41 g) and glacial acetic acid (150 g) were added in a 500 mL three-necked flask, the flask was placed in an oil bath for stirring, a temperature was risen to 50° C., phenylhydrazine (16 g) was slow added dropwise at 50° C., the adding time lasted for 1h, and after adding was completed, the temperature was risen to 80° C. for reaction for 8 h. The reaction was monitored through the point TLC plate, the raw materials were completely consumed, then the reaction was stopped; after the temperature was cooled to room temperature, the ethanol (200 mL) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-7 (34 g, purity 95%) as shown in the following reaction equation was obtained.

A Specific Reaction Equation VII was Shown as Follows.

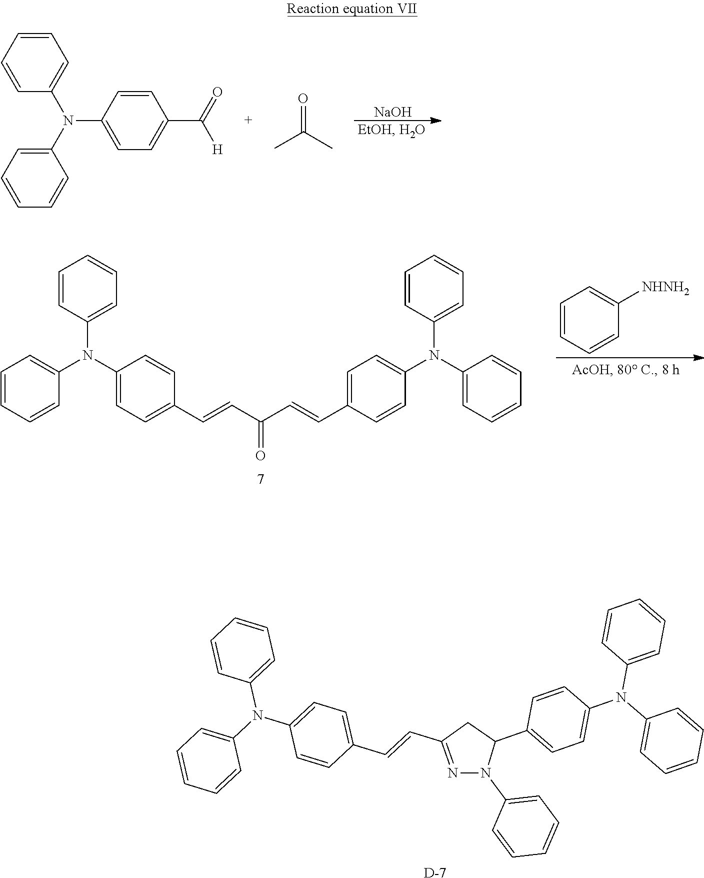

A sensitizer compound structure in the formula above was only used as an example, a benzene ring might carry substituents such as halogen, alkyl with a carbon chain length being C₁-C₈, and alkoxy with a carbon chain length being C₁-C₄, and the number of substituents might be one, or 1-5.

Various components were mixed in proportion according to formulas of Table 1 and Table 2 below; 60 parts of a solvent was added; the solvent suitable for preparing a coating adhesive solution might be acetone, butanone, methanol, ethanol, isopropanol, toluene, etc.; and then the mixture was fully stirred until completely dissolved, so as to prepare a resin composition solution with a solid content being 40%. The resin composition solution was allowed to stand for 30 min, full defoaming was performed, the resin composition solution was uniformly coated on a surface of a PET support film with a thickness being 16 um by using a coating machine, and was placed in a 90° C. oven to bake for 10 min, so as to form a dry film resist layer with a thickness being 25 μm, and blue green was shown under a yellow lamp. Then a polyethylene film protective layer with a thickness being 20 μm was laminated to the surface, so as to obtain a photosensitive dry film with a 3-layer structure.

TABLE 1

| | | Embodiment | | | | | | | | | |
|---|---|---|---|---|---|---|---|---|---|---|---|
| Composition (weight %) | | 1 | 2 | 3 | 4 | 5 | 6 | 7 | 8 | 9 | 10 |
| Alkali-soluble resin | A-1 | 53 | 53 | 53 | 53 | 53 | 53 | 53 | 45 | 65 | 70 |
| | A-2 | | | | | | | | | | |
| | A-3 | | | | | | | | | | |
| Photopolymerization | B-1 | 6 | 6 | 6 | 6 | 6 | 6 | 6 | 5 | 4 | 6 |
| monomer | B-2 | 26 | 26 | 26 | 26 | 26 | 26 | 26 | 32 | 16 | 11 |
| | B-3 | 5 | 5 | 5 | 5 | 5 | 5 | 5 | 5 | 4 | 4 |
| | B-4 | 5 | 5 | 5 | 5 | 5 | 5 | 5 | | 5 | 6.5 |
| | B-5 | | | | | | | | 7 | | |
| Photoinitiator | C-1 | 4 | 4 | 4 | 4 | 4 | 4 | 4 | 2 | 5 | 1.5 |
| | C-2 | | | | | | | | | | |
| Sensitizer | D-1 | 0.2 | | | | | | | 0.01 | | 0.2 |
| | D-2 | | 0.25 | | | | | | | | |
| | D-3 | | | 0.25 | | | | | | | |
| | D-4 | | | | 0.25 | | | | | | |
| | D-5 | | | | | 0.25 | | | | 0.5 | |
| | D-6 | | | | | | 0.25 | | | | |
| | D-7 | | | | | | | 0.25 | | | |
| | D-8 | | | | | | | | | | |
| | D-9 | | | | | | | | | | |
| Additive | F-1 | 0.002 | 0.002 | 0.002 | 0.002 | 0.002 | 0.002 | 0.002 | 0.001 | 0.005 | 0.002 |
| | F-2 | 0.05 | 0.05 | 0.05 | 0.05 | 0.05 | 0.05 | 0.05 | 0.05 | 0.05 | 0.1 |
| | F-3 | 0.5 | 0.5 | 0.5 | 0.5 | 0.5 | 0.5 | 0.5 | 2.4 | 0.445 | 0.5 |
| | F-4 | 0.248 | 0.198 | 0.198 | 0.198 | 0.198 | 0.198 | 0.198 | 1.539 | | 0.198 |

TABLE 2

| | | Embodiment | | | | Comparative example | | |
|---|---|---|---|---|---|---|---|---|
| Composition (weight %) | | 11 | 12 | 13 | 14 | 1 | 2 | 3 |
| Alkali-soluble resin | A-1 | | | 53 | 53 | 53 | 53 | 53 |
| | A-2 | 53 | | | | | | |
| | A-3 | | 53 | | | | | |
| Photopolymerization | B-1 | 6 | 6 | 6 | 6 | 6 | 6 | 6 |
| monomer | B-2 | 26 | 26 | 26 | 26 | 26 | 26 | 26 |
| | B-3 | 5 | 5 | 5 | 5 | 5 | 5 | 5 |
| | B-4 | 5 | 5 | 5 | 5 | 5 | 4.5 | 5 |
| | B-5 | | | | | | | |
| Photoinitiator | C-1 | 4 | 4 | 3 | | 4 | 4 | 4 |
| | C-2 | | | 1 | 4 | | | |
| Sensitizer | D-1 | 0.2 | 0.2 | 0.2 | 0.2 | | | |
| | D-2 | | | | | | | |
| | D-3 | | | | | | | |
| | D-4 | | | | | | | |
| | D-5 | | | | | | | |
| | D-6 | | | | | | | |
| | D-7 | | | | | | | |
| | D-8 | | | | | 0.25 | 0.65 | |
| | D-9 | | | | | | | 0.25 |
| Additive | F-1 | 0.002 | 0.002 | 0.002 | 0.002 | 0.002 | 0.02 | 0.02 |
| | F-2 | 0.05 | 0.05 | 0.05 | 0.05 | 0.05 | 0.05 | 0.05 |
| | F-3 | 0.5 | 0.5 | 0.5 | 0.5 | 0.5 | 0.5 | 0.5 |
| | F-4 | 0.248 | 0.248 | 0.248 | 0.248 | 0.198 | 0.28 | 0.18 |

The alkali-soluble resin, the photopolymerization monomer, the photoinitiator, the sensitizer, and the additive respectively were as follows.
A-1: methacrylic acid:methyl methacrylate:styrene:benzyl methacrylate=25:15:40:20, an acid value was 163 mg KOH/g, a weight-average molecular weight measured by a GPC was 50000 g/mol, a molecular weight distribution (PID) was 1.8, and a conversion rate was 97.0%.
A-2: methacrylic acid:methyl methacrylate:styrene:benzyl methacrylate=25:15:40:20, an acid value was 163 mg KOH/g, a weight-average molecular weight measured by a GPC was 100000 g/mol, a molecular weight distribution (PID) was 2.0, and a conversion rate was 97.0%.
A-3: methacrylic acid:methyl methacrylate:styrene=25:50:25, an acid value was (120-250) mg KOH/g, a weight-average molecular weight measured by a GPC was 50000 g/mol, a molecular weight distribution (PID) was 2.0, and a conversion rate was 95%.
B-1: (4) ethoxylated bisphenol A diacrylate (Miwon Special Chemical), in a structural formula B1 corresponding to the photopolymerization monomer, n₁+n₂=0, m₁+m₂=4, and R₁ was CH₃.
B-2: (10) ethoxylated bisphenol A diacrylate (Miwon Special Chemical), in a structural formula B1 corresponding to the photopolymerization monomer, n₁+n₂=0, m₁+m₂=10, and R₁ was CH₃.
B-3: (6) ethoxy propylene glycol (700) dimethacrylate (Miwon Special Chemical), in a structural formula B3 corresponding to the photopolymerization monomer, b₂=12, a₂+c₂=6, and R₁ was CH₃.
B-4: (3) ethoxylated trimethylolpropane trimethacrylate (Miwon Special Chemical), in a structural formula B4 corresponding to the photopolymerization monomer, a₃=1, and R₁═CH₃.
B-5: (4) ethoxylated nonylphenol methacrylate (Miwon Special Chemical).
C-1: 2,2′-Bis(2-chlorophenyl)-4,4′,5,5′-tetraphenyl diimidazole BCIM (Changzhou Tronly Electronic Materials Co. Ltd.).
C-2: Benzoyl dimethyl ether (also referred to as a,a-dimethoxy-a-phenylacetophenone, Shanghai Macklin Biochemical Materials Co., Ltd.).
D-1: 1-phenyl-3-(9-anthracenylvinyl)-5-(9-anthracenyl)pyrazoline, as shown in the synthesis example D-1.
D-2: 1-phenyl-3-(2-fluorenyl)-5-(9-anthracenyl)pyrazoline, as shown in the synthesis example D-2.
D-3: 1-phenyl-3-phenyl-5-(9-anthracenyl)pyrazoline, as shown in the synthesis example D-3.
D-4: 1-phenyl-3-biphenyl-5-(9-anthracenyl)pyrazoline, as shown in the synthesis example D-4.
D-5: 1-phenyl-3-(2-naphthyl)-5-(4-diphenylaminophenyl)pyrazoline, as shown in the synthesis example D-5.
D-6: 1-phenyl-3-(3-thienyl)-5-(4-diphenylaminophenyl)pyrazoline, as shown in the synthesis example D-6.
D-7: 1-phenyl-3-(4-diphenylaminophenylvinyl)-5-(4-diphenylaminophenyl)pyrazoline, as shown in the synthesis example D-7.
D-8: 9,10-dibutoxyanthracene (DBA).
D-9: 1-phenyl-3-(4-methoxy-styryl)-5-(4-methoxy-phenyl)pyrazoline (a sensitizer shown in a formula (1) reported in Patent CN104111583, R=methoxy).
F-1: 4-tert-butylcatechol
F-2: Brilliant green pigment (Shanghai J&K Chemical Technology Co., Ltd.)
F-3: Leucocrystal violet (Shanghai J&K Chemical Technology Co., Ltd.)
F-4: P-methyl benzenesulfonamide (Shanghai TCI Chemicals)

A sample preparation method (including film lamination, exposure, development, copper plating), a sample evaluation method, and an evaluation result of Embodiments 1-14 and Comparative examples 1-3 were described below.

### (1) Sample Preparation Method

[Film lamination]

A copper surface of a copper clad laminate was polished by using a grinding machine, and washing and drying were performed to obtain a bright and fresh copper surface. A pressure roller temperature of a laminator was set to 110° C., a convey speed was 1.5m/min, after a PE protective film on the surface of the photosensitive dry film obtained in the above embodiments and the comparative examples was removed, and the photosensitive dry film was thermally laminated to the copper clad laminate under a standard pressure, so as to obtain a laminated sample.

### [Exposure]

The laminated sample was allowed to stand for over 15 min, a resolution ratio and adhesion performance were tested, exposure was performed by using a Japan Adtec exposure machine, a model was IP-6, exposure was performed by using a LDI exposure machine with a wavelength being 405 nm, a photosensitivity test was performed by using a stouffer 41-order exposure ruler, and the number of exposure frames was controlled between 13 and 17.

### [Development]

The exposed sample was allowed to stand for over 15 min, a development temperature was 30° C., a pressure was 1.2 Kg/cm², a development solution was 1% wt sodium carbonate solution, a development time was 1.5-2.0 times of a minimum development time, and washing and drying were performed after development. A minimum time required for completely dissolving a resist layer on an unexposed portion was used as the minimum development time.

### [Etching]

A developed copper plate was etched, an etching solution was copper chloride, an etching speed was 1.0 m/min, an etching temperature was 48° C., a spray pressure was 1.5 bar, a specific weight was 1.3 g/mL, acidity was 2 mol/L, with 140 g/L of copper ions, and a model of an etching machine was Dongguan Universe GL181946.

[Film removal]

A film removal solution was NaOH with a concentration being 3.0 wt %, a temperature was 50° C., a pressure was 1.2 Kg/cm², a film removal time was 1.5-2.0 times of a minimum film removal time, and washing and drying were performed after film removal.

Evaluation method
[Evaluation of photosensibility]

The laminated sample was allowed to stand for over 15 min, exposure was performed by using an Adtec IP-6 405 nm LDI exposure machine, a photosensibility test was performed by using a stouffer 41-order exposure ruler, after exposure, a 1% wt sodium carbonate solution was sprayed at 30° C., a development time is 2.0 times of the minimum development time, such that the unexposed portion was removed. After these operations, a cured film formed by solids of components in the dry film resist was formed a copper surface of a substrate. The photosensibility of a photosensitive resin composition was evaluated by an exposure amount (mJ/cm²) at which the number of residual segments of a stage-type exposure meter obtained as the cured film became 15 frames. If the numerical value was smaller, it indicated that the photosensibility was better.

Determination basis:
o: 20-30 mJ/cm²
Δ: 30-50 mJ/cm²
x: >50 mJ/cm².
   [Evaluation of resolution ratio]

Exposure was performed by using a mask having a routing pattern with widths of an exposed portion and the unexposed portion being 1:1, development was performed with 2 times of the minimum development time, then a minimum mask width at which a cured resist line was normally formed was used as the value of the resolution ratio, a two-dimensional imager or a Scanning Electron Microscope (SEM) was used for observation, and if a number read was smaller, it indicated that the resolution ratio was more excellent.

[Evaluation of adhesion]

By laminating the photosensitive dry film resist on the copper plate through a hot pressed film, exposure was performed by using a mask having a routing pattern with widths of an exposed portion and the unexposed portion being n:400, corresponding sensitivity was 15 frames, development was performed with 2 times of the minimum development time, then a magnifier was used for observation, the minimum mask width at which the intact cured resist line was formed was used as the value of adhesion, and if the number read was smaller, it indicated that the adhesion is more excellent.

[Side shape evaluation]

After a PE film of the manufactured photosensitive dry film resist was removed, dry films were laminated on the copper plate by using a heated pressure roller. Here, exposure was performed by using the mask having the routing pattern with widths of the exposed portion and the unexposed portion being n:400, exposure energy was 15 frames corresponding to the sensitivity, development was performed with 2.0 times of the minimum development time, then a dry film image was obtained, and the SEM was used to take a side view of a dry film with a line width being 15 um by magnifying 1000 times.

Determination basis:
o: Head sections of the dry films were rectangular.
Δ: Some head sections of the dry films were in an inverted trapezoidal shape.
x: The head sections of the dry films were shown to be heavily inverted trapezoidal shaped or the bottom was hollowed out, or an obvious crack phenomenon occurred in a sidewall.
   [Evaluation of initiator migration]

The dry film resist laminated with the polyethylene film protective layer on the surface was placed at 30° C. for 48h, then the polyethylene film protective layer on the surface of the resist was taken down, an ultraviolet spectrophotometer was used to detect an ultraviolet absorption spectrum of the polyethylene film protective layer, and it was detected that a wavelength was 380-450 nm.

Determination basis:
o: In the corresponding ultraviolet absorption spectrum, the wavelength was 380-450 nm, and no significant absorption peaks were seen.
Δ: In the corresponding ultraviolet absorption spectrum, the wavelength was 380-450 nm, weak absorption peaks were seen, and a molar absorption coefficient of a maximum absorption peak was less than 0.01.
x: In the corresponding ultraviolet absorption spectrum, the wavelength was 380-450 nm, significant absorption peaks were seen, and the molar absorption coefficient of the maximum absorption peak was greater than 0.01.
(3) Evaluation results for performance such as photosensibility, resolution ratio, adhesion, side shape, initiator migration, and the like were shown in Table 3 and Table 4.

TABLE 3

| Evaluation | Embodiment | | | | | | | | | |
|---|---|---|---|---|---|---|---|---|---|---|
| method | 1 | 2 | 3 | 4 | 5 | 6 | 7 | 8 | 9 | 10 |
| 405 nm | 25 | 24 | 27 | 26 | 20 | 22 | 23 | 30 | 20 | 40 |
| photosensibility | | | | | | | | | | |
| mJ/cm² | | | | | | | | | | |
| Resolution | 10 | 9 | 9 | 9 | 11 | 10 | 9 | 8 | 12 | 20 |
| ratio/um | | | | | | | | | | |
| Adhesion/um | 10 | 9 | 11 | 10 | 11 | 11 | 10 | 12 | 12 | 20 |
| Side shape | ∘ | ∘ | ∘ | ∘ | ∘ | ∘ | ∘ | ∘ | ∘ | Δ |
| Whether | ∘ | ∘ | ∘ | ∘ | ∘ | ∘ | ∘ | ∘ | ∘ | ∘ |
| initiator | | | | | | | | | | |
| migration | | | | | | | | | | |
| occurred | | | | | | | | | | |

TABLE 4

| Evaluation | Embodiment | | | | Comparative example | | |
|---|---|---|---|---|---|---|---|
| method | 11 | 12 | 13 | 14 | 1 | 2 | 3 |
| 405nm | 25 | 25 | 27 | 30 | 100 | 60 | 30 |
| photosensibility | | | | | | | |
| mJ/cm2 | | | | | | | |
| Resolution | 25 | 18 | 14 | 20 | 12 | 15 | 22 |
| ratio/um | | | | | | | |
| Adhesion/um | 10 | 22 | 13 | 20 | 15 | 12 | 22 |
| Side shape | Δ | Δ | Δ | Δ | Δ | Δ | × |
| Whether | ∘ | ∘ | ∘ | ∘ | × | × | ∘ |
| initiator | | | | | | | |
| migration | | | | | | | |
| occurred | | | | | | | |

By comparing Embodiments 1-14 with Comparative examples 1-3, it might be found that the dry film resists that had excellent key comprehensive performance in terms of photosensibility, resolution ratio, adhesion performance, side shape, and initiator migration and were suitable for manufacturing high-order PCBs such as carrier boards and SLPs were all obtained in the embodiments.

In Comparative example 1, an initiator system was a hexaarylbiimidazole derivative combined with anthracene (DBA). The initiator system was an initiator system that was generally used in the dry film resist for manufacturing the high-order PCBs such as carrier boards and SLPs. When the addition of the DBA was equivalent to that of the sensitizer in the embodiments, the sensitivity of the corresponding dry film resist was very low, and an undesirable phenomenon of migration of the DBA towards a polyethylene protective film occurred.

In Comparative example 2, on the basis of Comparative example 1, the addition of the DBA was further increased, although the sensitivity was improved, the migration of the DBA towards the polyethylene protective film was more significant.

In Comparative example 3, a conventional pyrazoline compound reported in patents was used.

Although the photosensibility was good, the analysis and adhesion capacities of the corresponding dry film resist were still poor by using the initiator system, even combined with the high-analysis alkali-soluble resin and photopolymerization monomer, failing to meet performance requirements of the dry film resist for manufacturing the high-order PCBs such as carrier boards and SLPs.

From the above descriptions, it might be seen that, the embodiments of the present disclosure implemented the following technical effects. By introducing the anthracene and/or triarylamine in the pyrazoline structure, structure optimization was performed on high-order PCBs such as carrier boards and SLPs by using an anthracene and triarylamine sensitizer commonly-used in the dry film resist. Since the imidazoline compound had good photosensitivity, the compound had higher photosensitivity to an LDI 405 nm laser light source than the anthracene and triarylamine sensitizer, the modified sensitizer compound of the present application generally had a large rr conjugated structure, and such large conjugated electron effect caused a red shift in a maximum absorption wavelength of a modified sensitizer compound to further approach the wavelength of the 405 nm laser light source, such that in the present application, by introducing the pyrazoline structure in particular anthracene and triarylamine molecules, the photosensitivity of the obtained dry film resist to the LDI 405 nm laser light source could be significantly improved.

In the present application, for the sensitizer compound obtained through optimization and modification, the structure thereof simultaneously contained a pyrazoline ring structure and the anthracene or triarylamine structure, the optimized and modified sensitizer compound had a double active group, and the photosensitivity of the pyrazoline structure and the high-precision analysis capacity of the anthracene or triarylamine structure were effectively combined, such that the obtained dry film resist could improve the production efficiency of manufacturing high-order PCB clients such as carrier boards and SLPs while guaranteeing the high-precision analysis capacity.

Synthesis of sensitizer compound D-21 Raw materials of 4-acetylbiphenyl (117 g), 4-methoxybenzaldehyde (68 g), and ethanol (200 mL) were added in a 500 mL three-necked flask, the flask was placed in a water bath at room temperature, stirring was performed for 15 min, after the raw materials were dissolved, a 40% NaOH aqueous solution (40 g) was added dropwise to the flask, adding time was 1h, after the adding was completed, stirring was continuously performed at room temperature to react for 8h, a reaction was monitored through a point TLC plate, and after the raw material 4-methoxybenzaldehyde was completely consumed, the reaction was stopped. Filtration under a reduced pressure was performed on a suspended solid obtained in the reaction, an obtained solid crude product was dispersed in small amount of ethanol (150 mL), after 30 min of stirring at room temperature, suction filtration was performed to collect a solid, drying was performed by using a rotary evaporator, small amount of a solvent encapsulated in the solid was removed, and then an intermediate product compound 21 (145 g, purity 92%) as shown in the following reaction equation was obtained.

The intermediate product compound 21 (145 g) and glacial acetic acid (300 g) were added in a 500 mL three-necked flask, the flask was placed in an oil bath for stirring, a temperature was risen to 50° C. phenylhydrazine (95 g) was slow added dropwise at 50° C., the adding time lasted for 1h, and after an adding time was completed, the temperature was risen to 80° C. for reaction for 8 h. The reaction was monitored through the point TLC plate, the raw materials were completely consumed, then the reaction was stopped; after the temperature was cooled to room temperature, the ethanol (300 ml) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-21 (122 g, purity 95.5%) as shown in the following reaction equation was obtained.

A specific Reaction equation was shown as follows.

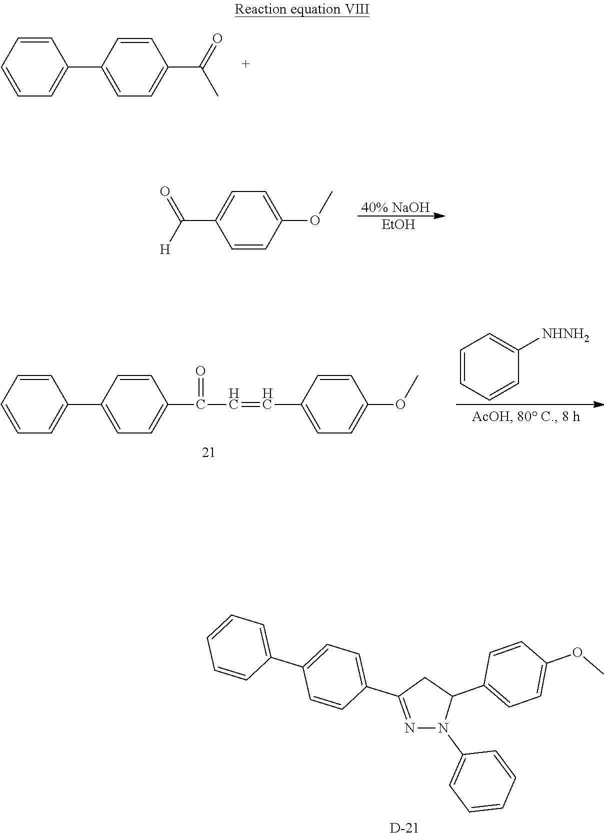

A sensitizer compound structure in the formula above was only used as an example, a benzene ring and a biphenyl ring might carry substituents such as halogen, C₁-C₈ alkyl, and C₁-C₄ alkoxy, and the number of substituents might be one, or 1-5.

### Synthesis of Sensitizer Compound D-22

Raw materials of 4-biphenylcarboxaldehyde (182 g), acetone (29 g), and ethanol (300 mL) were added in a 1000 mL three-necked flask, the flask was placed in a water bath at room temperature, stirring was performed for 15 min, after the raw materials were dissolved, a 10% NaOH aqueous solution (480 g) was added dropwise to the flask, adding time was 2h, after the adding was completed, stirring was continuously performed at room temperature to react for 8h, a reaction was monitored through a point TLC plate, and after the reaction had ceased to change, the reaction was stopped. Filtration under a reduced pressure was performed on a suspended solid obtained in the reaction, an obtained solid crude product was dispersed in small amount of ethanol (150 mL), after 30 min of stirring at room temperature, suction filtration was performed to collect a solid, drying was performed by using a rotary evaporator, small amount of a solvent encapsulated in the solid was removed, and then an intermediate product compound 22 (180 g, purity 90%) as shown in the following reaction equation was obtained.

The intermediate product compound 22 (120 g) and glacial acetic acid (300 g) were added in a 500 mL three-necked flask, the flask was placed in an oil bath for stirring, a temperature was risen to 50° C., phenylhydrazine (55 g) was slow added dropwise at 50° C., the adding time lasted for 1h, and after an adding time was completed, the temperature was risen to 80° C. for reaction for 8 h. The reaction was monitored through the point TLC plate, the raw materials were completely consumed, then the reaction was stopped; after the temperature was cooled to room temperature, the ethanol (300 ml) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-22 (101 g, purity 96%) as shown in the following reaction equation was obtained.

A specific Reaction equation was shown as follows.

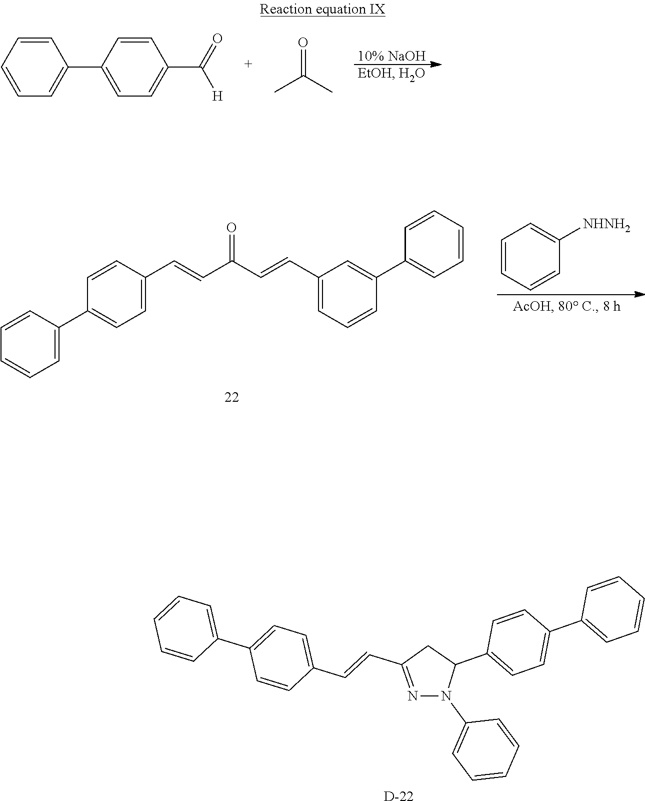

A sensitizer compound structure in the formula above was only used as an example, a benzene ring and a biphenyl ring might carry substituents such as halogen, C₁-C₈ alkyl, and C₁-C₄ alkoxy, and the number of substituents might be one, or 1-5.

### Synthesis of Sensitizer Compound D-23

Raw materials of 9-acridinecarboxaldehyde (124 g), acetone (14 g), and ethanol (200 mL) were added in a 500 mL three-necked flask, the flask was placed in a water bath at room temperature, stirring was performed for 15 min, after the raw materials were dissolved, a 10% NaOH aqueous solution (240 g) was added dropwise to the flask, adding time was 1h, after the adding was completed, stirring was continuously performed at room temperature to react for 8h, a reaction was monitored through a point TLC plate, and after the reaction had ceased to change, the reaction was stopped. Filtration under a reduced pressure was performed on a suspended solid obtained in the reaction, an obtained solid crude product was dispersed in small amount of ethanol (150 mL), after 30 min of stirring at room temperature, suction filtration was performed to collect a solid, drying was performed by using a rotary evaporator, small amount of a solvent encapsulated in the solid was removed, and then an intermediate product compound 23 (94 g, purity 91%) as shown in the following reaction equation was obtained.

The intermediate product compound 23 (80g) and glacial acetic acid (200 g) were added in a 500 mL three-necked flask, the flask was placed in an oil bath for stirring, a temperature was risen to 50° C., phenylhydrazine (34g) was slow added dropwise at 50° C., the adding time lasted for 1h, and after an adding time was completed, the temperature was risen to 80° C. for reaction for 8 h. The reaction was monitored through the point TLC plate, the raw materials were completely consumed, then the reaction was stopped; after the temperature was cooled to room temperature, the ethanol (200 mL) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-23 (57 g, purity 95%) as shown in the following reaction equation was obtained.

A specific Reaction equation was shown as follows.

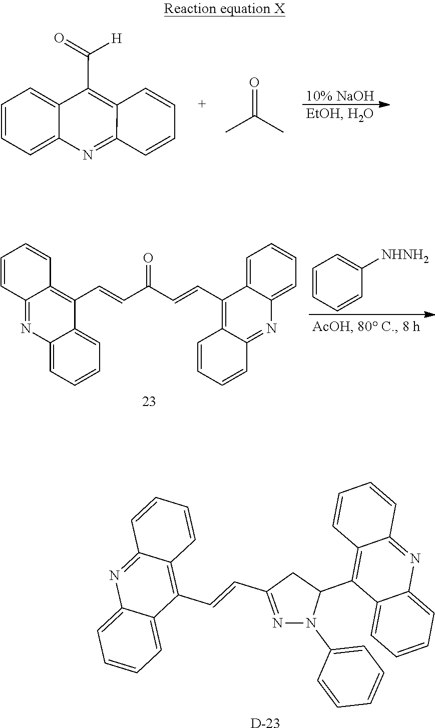

A sensitizer compound structure in the formula above was only used as an example, a benzene ring and an acridine ring might carry substituents such as halogen, C₁-C₈ alkyl, and C₁-C₄ alkoxy, and the number of substituents might be one, or 1-5.

### Synthesis of Sensitizer Compound D-24

Raw materials of 2-acetylfluorene (62 g), p-anisaldehyde (34 g), and ethanol (200 mL) were added in a 500 mL three-necked flask, the flask was placed in a water bath at room temperature, stirring was performed for 15 min, after the raw materials were dissolved, a 40% NaOH aqueous solution (20 g) was added dropwise to the flask, adding time was 1h, after the adding was completed, stirring was continuously performed at room temperature to react for 8h, a reaction was monitored through a point TLC plate, and after the raw material was completely consumed, the reaction was stopped. Filtration under a reduced pressure was performed on a suspended solid obtained in the reaction, an obtained solid crude product was dispersed in small amount of ethanol (150 mL), after 30 min of stirring at room temperature, suction filtration was performed to collect a solid, drying was performed by using a rotary evaporator, small amount of a solvent encapsulated in the solid was removed, and then an intermediate product compound 24 (78 g, purity 90%) as shown in the following reaction equation was obtained.

The intermediate product compound 24 (78 g) and glacial acetic acid (150 g) were added in a 500 mL three-necked flask, the flask was placed in an oil bath for stirring, a temperature was risen to 50° C., phenylhydrazine (40 g) was slow added dropwise at 50° C., the adding time lasted for 1h, and after an adding time was completed, the temperature was risen to 80° C. for reaction for 8 h. The reaction was monitored through the point TLC plate, the raw materials were completely consumed, then the reaction was stopped; after the temperature was cooled to room temperature, the ethanol (150 ml) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-24 (65 g, purity 96%) as shown in the following reaction equation was obtained.

A specific Reaction equation was shown as follows.

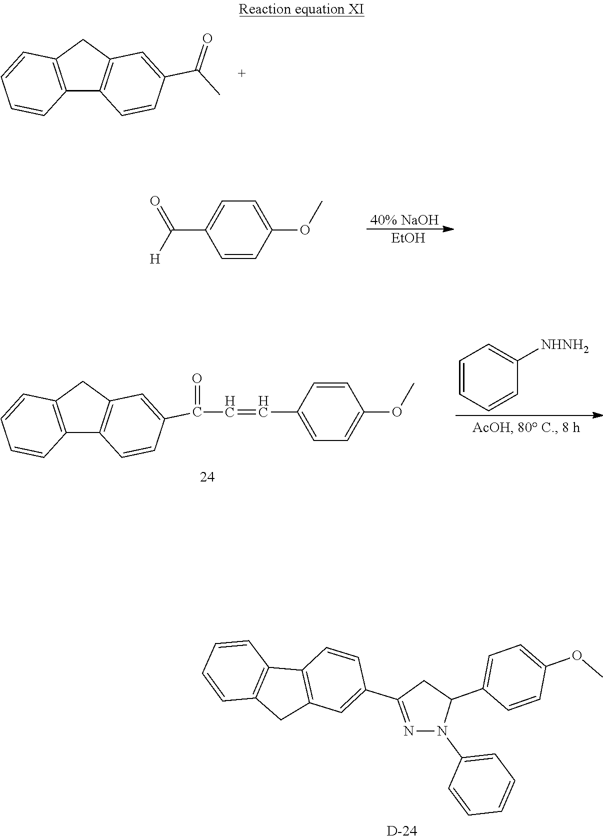

A sensitizer compound structure in the formula above was only used as an example, a benzene ring and a fluorene ring might carry substituents such as halogen, C₁-C₈ alkyl, and C₁-C₄ alkoxy, and the number of substituents might be one, or 1-5.

### Synthesis of Sensitizer Compound D-25

Raw materials of 4-acetylbiphenyl (58 g), benzo[b]thiophene-2-carboxaldehyde (40 g), and ethanol (150 mL) were added in a 500 mL three-necked flask, the flask was placed in a water bath at room temperature, stirring was performed for 15 min, after the raw materials were dissolved, a 40% NaOH aqueous solution (20 g) was added dropwise to the flask, adding time was 1h, after the adding was completed, stirring was continuously performed at room temperature to react for 8h, a reaction was monitored through a point TLC plate, and after the raw material benzo[b]thiophene-2-carboxaldehyde was completely consumed, the reaction was stopped.

Filtration under a reduced pressure was performed on a suspended solid obtained in the reaction, an obtained solid crude product was dispersed in small amount of ethanol (150 mL), after 30 min of stirring at room temperature, suction filtration was performed to collect a solid, drying was performed by using a rotary evaporator, small amount of a solvent encapsulated in the solid was removed, and then an intermediate product compound 25 (83 g, purity 93%) as shown in the following reaction equation was obtained.

The intermediate product compound 25 (83 g) and glacial acetic acid (200 g) were added in a 500 mL three-necked flask, the flask was placed in an oil bath for stirring, a temperature was risen to 50° C., phenylhydrazine (42 g) was slow added dropwise at 50° C., the adding time lasted for 1 h, and after an adding time was completed, the temperature was risen to 80° C. for reaction for 8 h. The reaction was monitored through the point TLC plate, the raw materials were completely consumed, then the reaction was stopped; after the temperature was cooled to room temperature, the ethanol (200 ml) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-25 (67 g, purity 95%) as shown in the following reaction equation was obtained.

A specific Reaction equation was shown as follows.

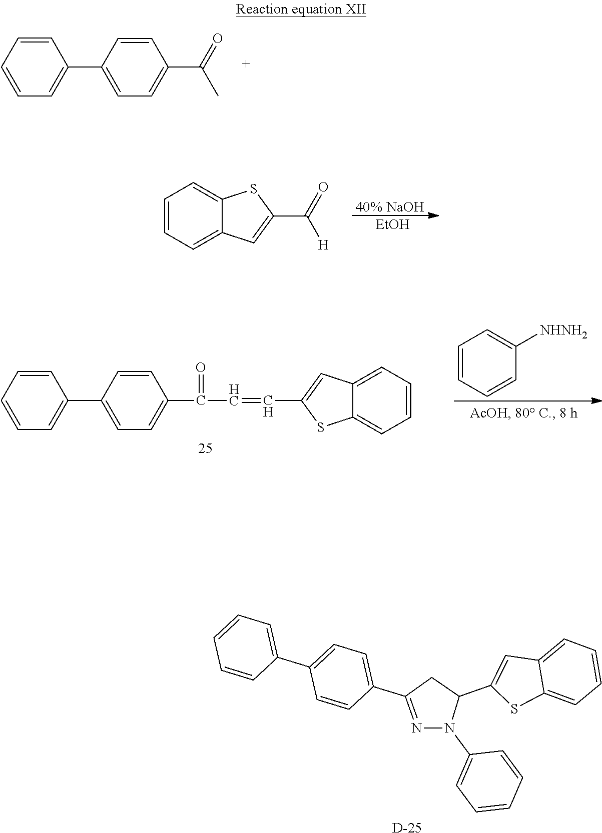

A sensitizer compound structure in the formula above was only used as an example, a benzene ring and a biphenyl ring might carry substituents such as halogen, C₁-C₈ alkyl, and C₁-C₄ alkoxy, and the number of substituents might be one, or 1-5. In addition, a benzothiophene heterocyclic ring might also be replaced with an electron-rich heterocyclic group such as furan, thiophene, indole, thiazole, benzofuran, benzothiazole, indene, anthracene, acridine, and aromatic amine.

### Synthesis of Sensitizer Compound D-26 as Shown in the Following Formula

Raw materials of 6-methoxy-2-naphthaldehyde (112 g), acetone (14 g), and ethanol (300 mL) were added in a 1000 mL three-necked flask, the flask was placed in a water bath at room temperature, stirring was performed for 15 min, after the raw materials were dissolved, a 10% NaOH aqueous solution (240 g) was added dropwise to the flask, adding time was 2h, after the adding was completed, stirring was continuously performed at room temperature to react for 8h, a reaction was monitored through a point TLC plate, and after the reaction had ceased to change, the reaction was stopped. Filtration under a reduced pressure was performed on a suspended solid obtained in the reaction, an obtained solid crude product was dispersed in small amount of ethanol (150 mL), after 30 min of stirring at room temperature, suction filtration was performed to collect a solid, drying was performed by using a rotary evaporator, small amount of a solvent encapsulated in the solid was removed, and then an intermediate product compound 26 (82 g, purity 91%) as shown in the following reaction equation was obtained.

The intermediate product compound 26 (82 g) and glacial acetic acid (200 g) were added in a 500 mL three-necked flask, the flask was placed in an oil bath for stirring, a temperature was risen to 50° C., phenylhydrazine (34 g) was slow added dropwise at 50° C., the adding time lasted for 1 h, and after an adding time was completed, the temperature was risen to 80° C. for reaction for 8 h. The reaction was monitored through the point TLC plate, the raw materials were completely consumed, then the reaction was stopped; after the temperature was cooled to room temperature, the ethanol (200 ml) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-26 (74 g, purity 96%) as shown in the following reaction equation was obtained.

A specific Reaction equation was shown as follows.

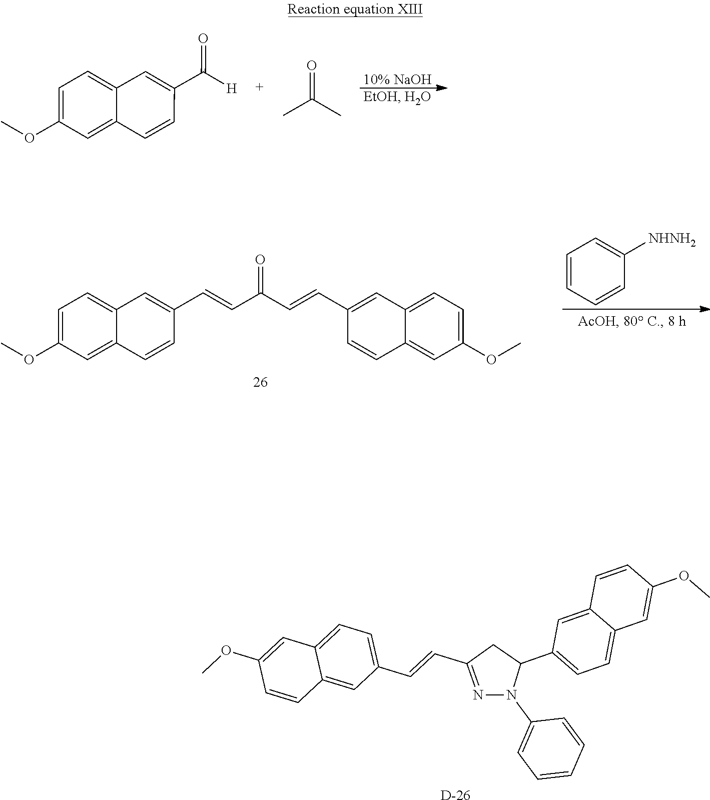

A sensitizer compound structure in the formula above was only used as an example, a benzene ring and a naphthalene ring might carry substituents such as halogen, C₁-C₈ alkyl, and C₁-C₄ alkoxy, and the number of substituents might be one, or 1-5. In addition, the naphthalene ring might also be replaced with a group such as an aromatic ring structure.

The alkali-soluble resin, the photopolymerization monomer, the photoinitiator, and the additive were mixed in proportion according to formulas of Table 5 and Table 6 below; 60 parts of a solvent was added; the solvent suitable for preparing a coating adhesive solution might be acetone, butanone, methanol, ethanol, isopropanol, toluene, etc.; and then the mixture was fully stirred until completely dissolved, so as to prepare a resin composition solution with a solid content being 40%. The resin composition solution was allowed to stand for 30 mn, full defoaming was performed, the resin composition solution was uniformly coated on a surface of a PET support film with a thickness being 16 um by using a coating machine, and was placed in a 90° C. oven to bake for 10 min, so as to form a dry film resist layer with a thickness being 25 μm, and blue green was shown under a yellow lamp. Then a polyethylene film protective layer with a thickness being 20 μm was laminated to the surface, so as to obtain a photosensitive dry film with a 3-layer structure.

TABLE 5

| | | Embodiment | | | | | | | | | |
|---|---|---|---|---|---|---|---|---|---|---|---|
| Composition (weight %) | | 15 | 16 | 17 | 18 | 19 | 20 | 21 | 22 | 23 | 24 |
| Alkali-soluble resin | A-21 | 54 | 54 | 54 | 54 | 54 | 54 | 45 | 57 | 65 | 54 |
| Photopolymerization | B-21 | 6 | 6 | 6 | 6 | 6 | 6 | 7 | 6 | 7 | 6 |
| monomer | B-22 | 25 | 25 | 25 | 25 | 25 | 25 | 30 | 25 | 17 | 25 |
| | B-23 | 3 | 3 | 3 | 3 | 3 | 3 | 3 | 3 | | 3 |
| | B-24 | 5 | 5 | 5 | 5 | 5 | 5 | 5 | 3 | | 5 |
| | B-25 | | | | | | | 5 | | 5 | |
| Photoinitiator | C-21 | 3.5 | 3.5 | 3.5 | 3.5 | 3.5 | 3.5 | 4.4 | 5 | 1 | 3.5 |
| | C-22 | | | | | | | | | | |
| | C-23 | | | | | | | | | | |
| Sensitizer | D-21 | 0.2 | | | | | | | | | 0.6 |
| | D-22 | | 0.2 | | | | | | | | |
| | D-23 | | | 0.2 | | | | 0.01 | | | |
| | D-24 | | | | 0.2 | | | | | | |
| | D-25 | | | | | 0.2 | | | 0.1 | | |
| | D-26 | | | | | | 0.2 | | | 0.5 | |
| | D-10 | | | | | | | | | | |
| | D-11 | | | | | | | | | | |
| Additive | F-21 | 0.04 | 0.04 | 0.04 | 0.04 | 0.04 | 0.04 | 0.04 | 0.04 | 0.04 | 0.04 |
| | F-22 | 1 | 1 | 1 | 1 | 1 | 1 | 0.5 | 0.5 | 1 | 1 |
| | F-23 | 0.06 | 0.06 | 0.06 | 0.06 | 0.06 | 0.06 | 0.05 | 0.06 | 0.06 | 0.06 |
| | F-24 | 2.2 | 2.2 | 2.2 | 2.2 | 2.2 | 2.2 | | 0.3 | 3.4 | 1.8 |

TABLE 6

| | | Embodiment | | | Comparative example | | | | |
|---|---|---|---|---|---|---|---|---|---|
| Composition (weight %) | | 25 | 26 | 27 | 4 | 5 | 6 | 7 | 8 |
| Alkali-soluble resin | A-21 | 54 | 54 | 70 | 54 | 54 | 54 | 54 | 57 |
| Photopolymerization | B-21 | 6 | 6 | 3 | 6 | 6 | 6 | 6 | 6 |
| monomer | B-22 | 25 | 25 | 17 | 25 | 25 | 25 | 25 | 25 |
| | B-23 | 3 | 3 | 1 | 3 | 3 | 3 | 3 | 3 |
| | B-24 | 5 | 5 | 2.5 | 5 | 5 | 5 | 5 | 5 |
| | B-25 | | | | | | | | |
| Photoinitiator | C-21 | 6 | 3.1 | 3.5 | 3.5 | 3.5 | 3.5 | 3.5 | |
| | C-22 | | 0.4 | | | | | | 0.7 |
| | C-23 | | | | | | | | 0.1 |
| Sensitizer | D-21 | 0.2 | 0.2 | 0.2 | | | | | |
| | D-22 | | | | | | | | |
| | D-23 | | | | | | | | |
| | D-24 | | | | | | | | |
| | D-25 | | | | | | | | |
| | D-26 | | | | | | | | |
| | D-10 | | | | 0.2 | | 0.8 | | |
| | D-11 | | | | | 0.2 | | | |
| Additive | F-21 | 0.04 | 0.04 | 0.04 | 0.04 | 0.04 | 0.04 | 0.04 | 0.04 |
| | F-22 | | 1 | 0.5 | 1 | 1 | 1 | 1 | 1 |
| | F-23 | 0.06 | 0.06 | 0.06 | 0.06 | 0.06 | 0.06 | 0.06 | 0.06 |
| | F-24 | 0.7 | 2.2 | 2.2 | 2.2 | 2.2 | 1.6 | 2.4 | 2.1 |

The alkali-soluble resin, the photopolymerization monomer, the photoinitiator, the sensitizer, and the additive respectively were as follows.
A-21: methacrylic acid:methyl methacrylate:butyl acrylate:styrene=25:40:7:28, an acid value was 131 mg KOH/g, a weight-average molecular weight measured by a GPC was 55000 g/mol, a molecular weight distribution (PID) was 1.8, and a conversion rate was 97.0%.
B-21: (4) ethoxylated bisphenol A diacrylate (Miwon Special Chemical)
B-22: (10) ethoxylated bisphenol A diacrylate (Miwon Special Chemical)
B-23: (6) ethoxy propylene glycol(700)dimethacrylate (Miwon Special Chemical)
B-24: (4) ethoxylated nonylphenol acrylate (Miwon Special Chemical)
B-25: (3) ethoxylated trimethylolpropane trimethacrylate (Miwon Special Chemical)
C-21: 2,2′-Bis(2-chlorophenyl)-4,4′,5,5′-tetraphenyl diimidazole BCIM (Changzhou Tronly Electronic Materials Co. Ltd.)
C-22: 9-phenylacridine (Changzhou Tronly Electronic Materials Co. Ltd.)
C-23: Anilinoacetic acid (Xiya Chemical)
D-21: 1-phenyl-3-biphenyl-5-(4-methoxyphenyl)pyrazoline, as shown in the synthesis example
D-21.
D-22: 1-phenyl-3-(biphenylvinyl)-5-biphenyl pyrazoline, as shown in the synthesis example D-22.
D-23: 1-phenyl-3-(9-acridinylvinyl)-5-(9-acridinyl)pyrazoline, as shown in the synthesis example D-23.
D-24: 1-phenyl-3-(2-fluorenyl)-5-(4-methoxyphenyl)pyrazoline, as shown in the synthesis example D-24.
D-25: 1-phenyl-3-(biphenyl)-5-(2-benzothienyl)pyrazoline, as shown in the synthesis example D-25.
D-26: 1-phenyl-3-(6-methoxy-2-naphthylvinyl)-5-(6-methoxy-2-naphthyl)pyrazoline, as shown in the synthesis example D-26.
D-10: 1-phenyl-3-(4-methoxyvinyl)-5-(4-methoxy-phenyl)pyrazoline, a sensitizer reported in Patent CN104111583 by Hitachi Chemical Industry Co., Ltd., R=methoxy.
D-11: 1-phenyl-3-(biphenyl)-5-(4-tert butyl phenyl)pyrazoline, a sensitizer reported in Patent CN101652715 by AsahiKASEl, R=tert butyl.
F-21: Brilliant green pigment (Shanghai J&K Chemical Technology Co., Ltd.)
F-22: Leucocrystal violet (Shanghai J&K Chemical Technology Co., Ltd.)
F-23: Phenyl tribromomethyl sulfone (Shanghai TCI Chemistry)
F-24: P-methyl benzenesulfonamide (Shanghai TCI Chemicals)

A sample preparation method (including film lamination, exposure, development, copper plating), a sample evaluation method, and an evaluation result of Embodiments 15-27 and Comparative examples 4-8 were described below.

(1) Sample preparation method
[Film lamination]

A copper surface of a copper clad laminate was polished by using a grinding machine, and washing and drying were performed to obtain a bright and fresh copper surface. A pressure roller temperature of a laminator was set to 110° C., a convey speed was 1.5m/min, after a PE protective film on the surface of the photosensitive dry film obtained in the above embodiments and the comparative examples was removed, and the photosensitive dry film was thermally laminated to the copper clad laminate under a standard pressure, so as to obtain a laminated sample.

### [Exposure]

The laminated sample was allowed to stand for over 15 min, a resolution ratio and adhesion performance were tested, exposure was performed by using a Japan Adtec exposure machine, a model was IP-6, exposure was performed by using a LDI exposure machine with a wavelength being 405 nm, a photosensitivity test was performed by using a stouffer 41-order exposure ruler, and the number of exposure frames was controlled between 14 and 20.

A para-position recognition performance test was performed on the sample on a domestic exposure machine, an LDI exposure machine from Wuxi Yingsu Semiconductor Company was used for exposure, the model of the exposure machine was IC2000, a wavelength was 405 nm, and the number of exposure frames was controlled between 14 and 20.

### [Development]

The exposed sample was allowed to stand for over 15 min, a development temperature was 30° C., a pressure was 1.2 Kg/cm², a development solution was 1% wt sodium carbonate solution, a development time was 1.5-2.0 times of a minimum development time, and washing and drying were performed after development. A minimum time required for completely dissolving a resist layer on an unexposed portion was used as the minimum development time.

### [Etching]

A developed copper plate was etched, an etching solution was copper chloride, an etching speed was 1.0 m/min, an etching temperature was 48° C., a spray pressure was 1.5 bar, a specific weight was 1.3 g/mL, acidity was 2 mol/L, with 140 g/L of copper ions, and a model of an etching machine was Dongguan Universe GL181946.

[Film removal]

A film removal solution was NaOH with a concentration being 3.0 wt %, a temperature was 50° C., a pressure was 1.2 Kg/cm², a film removal time was 1.5-2.0 times of a minimum film removal time, and washing and drying were performed after film removal.

Evaluation method
[Evaluation of photosensibility]

The laminated sample was allowed to stand for over 15 min, exposure was performed by using an Adtec IP-6 405 nm LDI exposure machine, a photosensibility test was performed by using a stouffer 41-order exposure ruler, after exposure, a 1% wt sodium carbonate solution was sprayed at 30° C., a development time is 2.0 times of the minimum development time, such that the unexposed portion was removed. After these operations, a cured film formed by solids of a photosensitive resin composition was formed a copper surface of a substrate. The photosensibility of a photosensitive resin composition was evaluated by an exposure amount (mJ/cm²) at which the number of residual segments of a stage-type exposure meter obtained as the cured film became 17 frames. If the numerical value was smaller, it indicated that the photosensibility was better.

Determination basis:
o: 10-20 mJ/cm²
Δ: 20-50 mJ/cm²
x: >50 mJ/cm².
   [Evaluation of resolution ratio]

Exposure was performed by using a mask having a routing pattern with widths of an exposed portion and the unexposed portion being 1:1, development was performed with 2 times of the minimum development time, then a minimum mask width at which a cured resist line was normally formed was used as the value of the resolution ratio, a two-dimensional imager or a Scanning Electron Microscope (SEM) was used for observation, and if a number read was smaller, it indicated that the resolution ratio was more excellent.

[Evaluation of adhesion]

By laminating the photosensitive dry film resist on the copper plate through a hot pressed film, exposure was performed by using a mask having a routing pattern with widths of an exposed portion and the unexposed portion being n:400, corresponding sensitivity was 17 frames, development was performed with 2 times of the minimum development time, then a magnifier was used for observation, the minimum mask width at which the intact cured resist line was formed was used as the value of adhesion, and if the number read was smaller, it indicated that the adhesion is more excellent.

[Para-position recognition on domestic LDI exposure machine]

An H-9300D LDI exposure machine from Yingsu Technology was used for exposure, exposure energy determinaton was performed by using a stouffer 41-frame exposure ruler, the number of exposure frames was 17; and while Face A was exposed, Face B was irradiated and colored with an UV LED, and then the exposure machine automatically used red light or yellow light to perform point recognition. If a radiation burning time is shorter, an image contrast after radiation was larger, and a radiation point boundary was clearer; and point recognition was able to be performed by using the red light or the yellow light after burning, it indicated that the para-position recognition performance on the domestic LDI exposure machine was more excellent.

Determination basis:
o: The irradiation coloring time was 0.5-2.0s, and after coloring, point recognition was able to be performed by using the red light or the yellow light.
Δ: The irradiation coloring time was 2.0-3.0s, and after coloring, point recognition was unable to be performed by using the red light, and only the yellow light could be used for recognition.
x: The irradiation coloring time was longer than 3.0s, and after coloring, point recognition was unable to be performed by using both the red light and the yellow light.
   [Side shape evaluation]

After a PE film of the manufactured photosensitive dry film resist was removed, dry films were laminated on the copper plate by using a heated pressure roller. Here, exposure was performed by using the mask having the routing pattern with widths of the exposed portion and the unexposed portion being n:400, development was performed with 2.0 times of the minimum development time, then a dry film image was obtained, and the SEM was used to take a side view of a dry film with a line width being 25 um by magnifying 1000 times.

Determination basis:
o: Head sections of the dry films were rectangular.
Δ: Some head sections of the dry films were in an inverted trapezoidal shape.
x: The head sections of the dry films were shown to be heavily inverted trapezoidal shaped or the bottom was hollowed out, or an obvious crack phenomenon occurred in a sidewall.
   [Evaluation of film removal speed]

A film removal speed was evaluated by testing a film removal time, and if the film removal time was shorter, the film removal speed was faster.
(3) Evaluation results for performance such as photosensibility, resolution ratio, adhesion, side shape, para-position recognition on the domestic LDI exposure machine, and the like were shown in Table 7 and Table 8.

TABLE 7

| | | Embodiment | | | | | | | | | |
|---|---|---|---|---|---|---|---|---|---|---|---|
| Evaluation method | | 15 | 16 | 17 | 18 | 19 | 20 | 21 | 22 | 23 | 24 |
| 405 nm | | 13 | 14 | 10 | 11 | 13 | 13 | 20 | 16 | 30 | 10 |
| photosensibility | | | | | | | | | | | |
| mJ/cm² | | | | | | | | | | | |
| Resolution | ST = 14/41 | 15 | 16 | 16 | 16 | 15 | 14 | 15 | 15 | 27 | 28 |
| ratio/um | ST = 17/41 | 16 | 17 | 17 | 17 | 16 | 16 | 15 | 16 | 25 | 25 |
| | ST = 20/41 | 18 | 19 | 19 | 17 | 18 | 17 | 18 | 17 | 22 | 24 |

| | | | | | | | | | | |
|---|---|---|---|---|---|---|---|---|---|---|
| Adhesion/um | 15 | 15 | 16 | 15 | 15 | 14 | 16 | 16 | 25 | 27 |
| Para-position | ∘ | ∘ | ∘ | ∘ | ∘ | ∘ | Δ | ∘ | Δ | ∘ |
| recognition on | | | | | | | | | | |
| domestic LDI | | | | | | | | | | |
| exposure machine | | | | | | | | | | |
| Side shape | ∘ | ∘ | ∘ | ∘ | ∘ | ∘ | ∘ | ∘ | Δ | Δ |

TABLE 8

| | | Embodiment | | | Comparative example | | | | |
|---|---|---|---|---|---|---|---|---|---|
| Evaluation method | | 25 | 26 | 27 | 4 | 5 | 6 | 7 | 8 |
| 405 nm | | 10 | 11 | 13 | 60 | 30 | 20 | 150 | 11 |
| photosensibility | | | | | | | | | |
| mJ/cm² | | | | | | | | | |
| Resolution | ST = 14/41 | 20 | 18 | 22 | 20 | 24 | × | 30 | 20 |
| ratio/um | ST = 17/41 | 22 | 20 | 25 | 22 | 26 | × | 35 | 22 |
| | ST = 20/41 | 22 | 22 | 27 | 22 | 28 | × | 35 | 25 |

| | | | | | | | | |
|---|---|---|---|---|---|---|---|---|
| Adhesion/um | 18 | 18 | 20 | 22 | 26 | × | 30 | 22 |
| Para-position | ∘ | ∘ | ∘ | ∘ | ∘ | ∘ | × | × |
| recognition on | | | | | | | | |
| domestic LDI | | | | | | | | |
| exposure machine | | | | | | | | |
| Side shape | Δ | Δ | Δ | Δ | Δ | × | × | Δ |

By comparing the embodiments with the comparative examples, it might be found that the dry film resists that had excellent key comprehensive performance in terms of photosensibility, resolution ratio, adhesion performance, side shape, and para-position recognition on the domestic LDI exposure machine and were able to be applied to the LDI exposure machine were all obtained in the embodiments.

In Comparative example 4 and Comparative example 5, a pyrazoline compound in the related art was used as the sensitizer. From experimental results, it was seen that experimental test data was more in line with data provided in the patent. Although the para-position recognition performance on the domestic LDI exposure machine was good, the photosensibility was obviously insufficient, and the resolution ratio and adhesion were also required to be further improved.

In Comparative example 6, in order to further improve the photosensibility, on the basis of Comparative example 4, the addition of the sensitizer was increased. However, from experimental results, it was seen that, after the use amount of the sensitizer was greatly increased, although the photosensibility was improved indeed, the adhesion greatly reduced, such that a dry film line did not adhere to the copper surface at all, and the color of the dry film become abnormally dark after exposure and development. It was assumed that the reason was because the resist was limited to surface curing, and the darkening of the surface caused a curing depth to be insufficient, resulting in a dry film pattern not being able to adhere to the copper surface at all after exposure.

In Comparative example 7, the initiator was hexaaromatic imidazole, but the sensitizer was not used. From embodiment results, it was seen that, the photosensitivity of the corresponding dry film resist was very poor, and performance such as analysis, adhesion and side shapes were all poor.

Comparative example 8 was a solution of a high sensitivity and high resolution dry film resist currently commonly used for manufacturing HDI inner layer boards. Experimental test results were consistent with PCB client test results, the photosensitivity was high at 405 nm, and resolution precision was excellent. However, in such formula, even though a certain amount of phenyl tribromomethyl sulfone was added to increase an image contrast before and after exposure, para-position recognition could not be performed on the domestic LDI exposure machine, causing the dry film resist using such formula system to be unable to be used on the domestic LDI exposure machine recently widely introduced by a PCB client. It was assumed that the reason was because an acridine initiator system used in such formula, after the initiator initiated photopolymerization, color development after exposure was too slow, and pattern contrast ratio before and after exposure was too weak, causing the domestic LDI exposure machine to be unable to perform para-position recognition.

From the above descriptions, it might be seen that, the embodiments of the present application implemented the following technical effects. By introducing some modified groups such as biphenyl, condensed ring groups, electron-rich heterocyclic or fused-heterocyclic rings, and the benzene ring with the amino into the pyrazoline compound, an entire molecular structure of the modified sensitizer compound as shown in the Structural formulas I and Structural formulas II is an electron-rich conjugated system, such electron-rich conjugation effect facilitates the promotion of red shift of an absorption spectrum of the sensitizer, and the absorption spectrum may extend to a visible region. A maximum absorption wavelength of the modified sensitizer is closer to an exposure light source with a wavelength being 405 nm, which is more sensitive to the exposure light source, such that the photosensitivity of the dry film resist to an LDI 405 nm exposure light source is improved, and the resolution ratio was significantly improved. Furthermore, in the present application, by selecting the particular sensitizer, the dry film resist was high in photosensitivity and resolution ratio, such that a pattern contrast ratio before and after exposure could be further enhanced, thereby more facilitating para-position recognition of domestic LDI exposure machines, so as to guarantee para-position precision of the sensitizer on the domestic LDI exposure machines.

### Synthesis of Sensitizer Compound D-27

Raw materials of 4-biphenylcarboxaldehyde (68 g), acetone (14 g), and ethanol (150 mL) were added in a 1000 mL three-necked flask, the flask was placed in a water bath at room temperature, stirring was performed for 15 min, after the raw materials were dissolved, a 10% NaOH aqueous solution (240 g) was added dropwise to the flask, adding time was 1h, after the adding was completed, stirring was continuously performed at room temperature to react for 8h, a reaction was monitored through a point TLC plate, and after the reaction had ceased to change, the reaction was stopped. Filtration under a reduced pressure was performed on a suspended solid obtained in the reaction, an obtained solid crude product was dispersed in small amount of ethanol (100 mL), after 30 min of stirring at room temperature, suction filtration was performed to collect a solid, drying was performed by using a rotary evaporator, small amount of a solvent encapsulated in the solid was removed, and then an intermediate product compound 27 (56 g, purity 90%) as shown in the following reaction equation was obtained.

The intermediate product compound 27 (56 g) and glacial acetic acid (150 g) were added in a 500 mL three-necked flask, the flask was placed in an oil bath for stirring, a temperature was risen to 50° C., phenylhydrazine (29 g) was slow added dropwise at 50° C., the adding time lasted for 0.5h, and after an adding time was completed, the temperature was risen to 80° C. for reaction for 8 h. The reaction was monitored through the point TLC plate, the raw materials were completely consumed, then the reaction was stopped; after the temperature was cooled to room temperature, the ethanol (150 ml) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-27 (52 g, purity 97%) as shown in the following reaction equation was obtained.

A specific reaction equation was shown as follows.

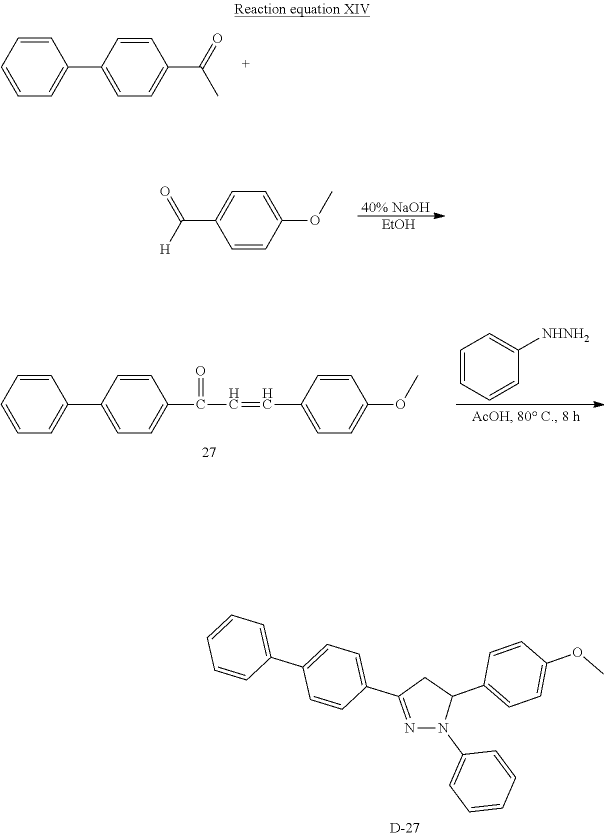

A sensitizer compound structure in the formula above was only used as an example, a benzene ring might carry substituents such as halogen, alkyl with a carbon chain length being C₁-C₈, and alkoxy with a carbon chain length being C₁-C₄, and the number of substituents might be one, or 1-5.

### Synthesis of Sensitizer Compound D-28

Raw materials of indole-3-carboxaldehyde (72 g), acetone (14 g), and ethanol (100 mL) were added in a 500 mL three-necked flask, the flask was placed in a water bath at room temperature, stirring was performed for 15 min, after the raw materials were dissolved, a 10% NaOH aqueous solution (240 g) was added dropwise to the flask, adding time was 1h, after the adding was completed, stirring was continuously performed at room temperature to react for 8h, a reaction was monitored through a point TLC plate, and after the reaction had ceased to change, the reaction was stopped. Filtration under a reduced pressure was performed on a suspended solid obtained in the reaction, an obtained solid crude product was dispersed in small amount of ethanol (200 mL), after 30 min of stirring at room temperature, suction filtration was performed to collect a solid, drying was performed by using a rotary evaporator, small amount of a solvent encapsulated in the solid was removed, and then an intermediate product compound 28 (63 g, purity 89%) as shown in the following reaction equation was obtained.

The intermediate product compound 28 (63 g) and glacial acetic acid (200 g) were added in a 500 mL three-necked flask, the flask was placed in an oil bath for stirring, a temperature was risen to 50° C., phenylhydrazine (33 g) was slow added dropwise at 50° C., the adding time lasted for 1 h, and after an adding time was completed, the temperature was risen to 80° C. for reaction for 8 h. The reaction was monitored through the point TLC plate, the raw materials were completely consumed, then the reaction was stopped; after the temperature was cooled to room temperature, the ethanol (200 ml) was added for dilution; the obtained suspended solid was stirred for 30 min, then filtration under a reduced pressure was performed, the obtained solid crude product was required to be further purified and dispersed in small amount of methanol (150 mL), after 30 min of stirring at room temperature, suction filtration was performed to collect a solid, drying was performed by using a rotary evaporator, small amount of a solvent encapsulated in the solid was removed, and then the sensitizer compound D-28 (55 g, purity 95%) as shown in the following reaction equation was obtained.

A specific reaction equation was shown as follows.

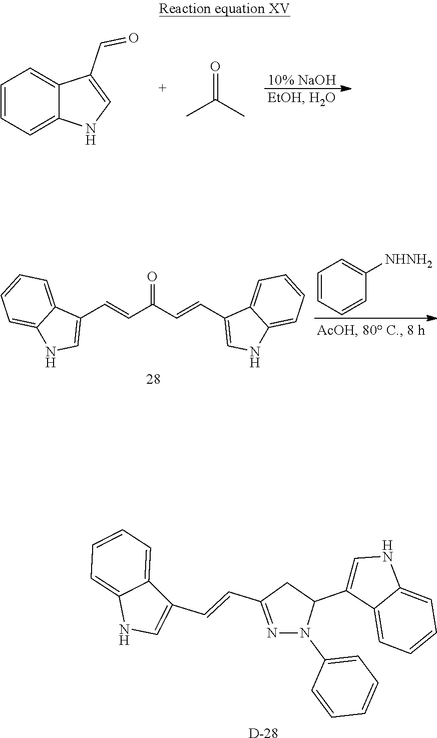

A sensitizer compound structure in the formula above was only used as an example, a benzene ring and an indole ring might carry substituents such as halogen, alkyl with a carbon chain length being C₁-C₈, and alkoxy with a carbon chain length being C₁-C₄, and the number of substituents might be one, or 1-5.

Various components were mixed in proportion according to formulas of Table 9 and Table 10 below; 60 parts of a solvent was added; the solvent suitable for preparing a coating adhesive solution might be acetone, butanone, methanol, ethanol, isopropanol, toluene, etc.; and then the mixture was fully stirred until completely dissolved, so as to prepare a solution with a solid content being 40%. The resin composition solution was allowed to stand for 30 min, full defoaming was performed, the resin composition solution was uniformly coated on a surface of a PET support film with a thickness being 16 um by using a coating machine, and was placed in a 90° C. oven to bake for 10 min, so as to form a dry film resist layer with a thickness being 38 μm, and blue green was shown under a yellow lamp. Then a polyethylene film protective layer with a thickness being 20 μm was laminated to the surface, so as to obtain a photosensitive dry film with a 3-layer structure.

TABLE 9

| | | Embodiment | | | | | | | | | | |
|---|---|---|---|---|---|---|---|---|---|---|---|---|
| Composition (weight %) | | 28 | 29 | 30 | 31 | 32 | 33 | 34 | 35 | 36 | 37 | 38 |
| Alkali-soluble resin | A-31 | 55 | 55 | 55 | 55 | 55 | 40 | 65 | 55 | 55 | 54 | 56 |
| Photopolymerization | B-31 | 10 | 10 | 10 | 10 | 10 | 12 | | 10 | 10 | 10 | 10 |
| monomer | B-32 | 10 | 10 | 10 | 10 | 10 | 8 | 15 | 10 | 10 | 10 | 10 |
| | B-33 | 6 | 6 | 6 | 6 | 6 | 20 | 8 | 6 | 6 | 6 | 6 |
| | B-34 | 8 | 8 | 8 | 8 | 8 | 9 | | 8 | 8 | 8 | 8 |
| | B-35 | 5 | 5 | 5 | 5 | 5 | 5 | 8 | 5 | 5 | 5 | 5 |
| | B-36 | | | | | | | | | | | |
| | B-37 | | | | | | | | | | | |
| | B-38 | | | | | | | | | | | |
| Photoinitiator | C-31 | 3.6 | 3.6 | 3.6 | 3.6 | 3.6 | 4 | 3 | 3.6 | 3.6 | 4.5 | 2 |
| | C-32 | | | | | | | | | | | |
| | C-33 | | | | | | | | | | | |
| | C-34 | | | | | | | | | | | |
| Sensitizer | D-21 | 0.2 | | | 0.2 | 0.2 | | | | | 0.2 | 0.2 |
| | D-27 | | | | | | 0.5 | | | | | |
| | D-22 | | 0.2 | | | | | | | | | |
| | D-23 | | | | | | | 0.01 | 0.2 | | | |
| | D-28 | | | 0.2 | | | | | | | | |
| | D-26 | | | | | | | | | 0.2 | | |
| | D-12 | | | | | | | | | | | |
| Compound that | E-1 | 0.05 | 0.05 | 0.05 | | | | | | | 0.05 | 0.05 |
| forms complex with | E-2 | | | | | | 0.05 | | | 0.5 | | |
| copper | E-3 | | | | | | | 0.2 | 0.01 | | | |
| | E-4 | | | | 0.05 | | | | | | | |
| | E-5 | | | | | 0.05 | | | | | | |
| Additive | F-31 | 0.04 | 0.04 | 0.04 | 0.04 | 0.04 | 0.04 | 0.04 | 0.04 | 0.04 | 0.04 | 0.04 |
| | F-32 | 1 | 1 | 1 | 1 | 1 | 0.41 | | 1 | 1 | 1 | 1 |
| | F-33 | 1.11 | 1.11 | 1.11 | 1.11 | 1.11 | 1 | 0.75 | 1.15 | 0.66 | 1.21 | 1.71 |

TABLE 10

| | | Embodiment | | | | | | | | Comparative example | | |
|---|---|---|---|---|---|---|---|---|---|---|---|---|
| Composition (weight %) | | 39 | 40 | 41 | 42 | 43 | 44 | 45 | 46 | 9 | 10 | 11 |
| Alkali-soluble resin | A-31 | 55 | 55 | 55 | 55 | 55 | 55 | 55 | 55 | 55 | 55 | 55 |
| Photopolymerization | B-31 | 10 | 10 | 18 | | | 10 | 11.5 | 10 | 12 | 11.5 | 18 |
| monomer | B-32 | 10 | 10 | 12 | 6 | 5 | 10 | 12 | 10 | 12 | 12 | |
| | B-33 | 6 | 6 | | 28 | 4 | 6 | 6 | 6 | 6 | 6 | |
| | B-34 | 8 | 8 | 4 | | | 8 | 8 | 8 | 8 | 8 | |
| | B-35 | 5 | 5 | 5 | 5 | 4 | 5 | 5 | 5 | 5 | 5 | 6 |
| | B-36 | | | | | | | | | | | 8 |
| | B-37 | | | | | | | | | | | 8 |
| | B-38 | | | | | 26 | | | | | | |
| Photoinitiator | C-31 | 3.6 | 3.6 | 3.6 | 3.6 | 3.6 | 3.6 | | 3.6 | | 3.6 | 3.6 |
| | C-32 | | | | | | | | 0.2 | | | |
| | C-33 | | | | | | | 0.6 | | 0.6 | | |
| | C-34 | | | | | | | 0.1 | | 0.1 | | |
| Sensitizer | D-21 | 0.3 | 0.5 | 0.2 | 0.2 | 0.2 | | | | | | |
| | D-27 | | | | | | | | | | | |
| | D-22 | | | | | | | | | | | |
| | D-23 | | | | | | | | | | | 0.2 |
| | D-28 | | | | | | | | | | | |
| | D-26 | | | | | | | | | | 0.2 | |
| | D-12 | | | | | | 0.2 | | | | | |
| Compound that | E-1 | 0.01 | 0.01 | 0.05 | 0.05 | 0.05 | 0.05 | | 0.05 | | | |
| forms complex with | E-2 | | | | | | | 0.05 | | | | 0.05 |
| copper | E-3 | | | | | | | | | | | |
| | E-4 | | | | | | | | | | | |
| | E-5 | | | | | | | | | | | |
| Additive | F-31 | 0.04 | 0.04 | 0.04 | 0.04 | 0.04 | 0.04 | 0.04 | 0.04 | 0.04 | 0.04 | 0.04 |
| | F-32 | 1 | 1 | 1 | 1 | 1 | 1 | 1 | 1 | 1 | 1 | 0.85 |
| | F-33 | 1.05 | 0.85 | 1.11 | 1.11 | 1.11 | 1.11 | 0.71 | 1.11 | 0.26 | 0.26 | 0.26 |

The alkali-soluble resin, the photopolymerization monomer, the photoinitiator, the sensitizer, the compound that forms a complex with copper, and the additive respectively were as follows.
A-31: methacrylic acid: methyl methacrylate: butyl methacrylate: butyl acrylate:styrene=22:40:20:11:7, an acid value was 127 mg KOH/g, a weight-average molecular weight measured by a GPC was 91000 g/mol, a molecular weight distribution (PID) was 2.0, and a conversion rate was 97.0%.
B-31: (10) ethoxylated bisphenol A dimethacrylate (Miwon Special Chemical), in a structural formula B1 corresponding to the photopolymerization monomer, m₁+m₂=10, n₁+n₂=0, and R₁ was methyl.
B-32: (10) ethoxylated (4) propoxylate bisphenol A dimethacrylate (Miwon Special Chemical), in a structural formula B1 corresponding to the photopolymerization monomer, m₁+m₂=10, n₁+n₂=4, and R₁ was methyl.
B-33: (6) ethoxy propylene glycol(700)dimethacrylate (Miwon Special Chemical), in a structural formula B6 corresponding to the photopolymerization monomer, a₆=6, b₆=12, and R₁=methyl.
B-34: (8) ethoxylated (4) propoxylate nonylphenol acrylate (Miwon Special Chemical), in a structural formula B5 corresponding to the photopolymerization monomer, a₅=8, b₅=4, and R₁═H.
B-35: (3) ethoxylated trimethylolpropane trimethacrylate (Miwon Special Chemical), in a structural formula B7 corresponding to the photopolymerization monomer, a₇=1, b₇=0, and R₁═H.
B-36: polyethylene glycol(400)dimethacrylate (Miwon Special Chemical).
B-37: (20) ethoxylated bisphenol A dimethacrylate (Miwon Special Chemical).
B-38: propylene glycol(400)dimethacrylate (Miwon Special Chemical).
C-31: 2,2′-Bis(2-chlorophenyl)-4,4′,5,5′-tetraphenyl diimidazole BCIM (Changzhou Tronly Electronic Materials Co. Ltd.).
C-32: 4,4′-bis(diethylamino) benzophenone
C-33: 9-phenylacridine (Changzhou Tronly Electronic Materials Co. Ltd.)
C-34: Anilinoacetic acid (Xiya Chemical)
D-21: 1-phenyl-3-biphenyl-5-(4-methoxyphenyl)pyrazoline, as shown in the synthesis example
D-21, with purity being 95%.
D-27: 1-phenyl-3-(4-methoxyvinyl)-5-(4-methoxyphenyl)pyrazoline, as shown in the synthesis example D-27.
D-22: 1-phenyl-3-(biphenylvinyl)-5-biphenyl pyrazoline, as shown in the synthesis example D-22, with purity being 95.5%.
D-23: 1-phenyl-3-(9-acridinylvinyl)-5-(9-acridinyl)pyrazoline, as shown in the synthesis example D-23, with purity being 94%.
D-28: 1-phenyl-3-(3-indolylvinyl)-5-(3-indolyl)pyrazoline, as shown in the synthesis example D-28.
D-26: 1-phenyl-3-(6-methoxy-2-naphthylvinyl)-5-(6-methoxy-2-naphthyl)pyrazoline, as shown in the synthesis example D-26, with purity being 96%.
D-12: 1-phenyl-3-(4-methoxyvinyl)-5-(4-methoxy-phenyl)pyrazoline, a sensitizer reported in Patent CN104111583 by Hitachi Chemical Industry Co., Ltd.
E-1: 2-mercapto-1H-benzo[d]iMidazole-4-carboxylic acid, belonging to the Structural formula IV.
E-2: 2-mercaptobenzothiazole, belonging to the Structural formula IV.
E-3: 6-thioguanine, belonging to the Structural formula IV.
E-4: Mercaptotriazole, belonging to the Structural formula III.
E-5: 2-(5-Mercaptotetrazole-1-yl)ethanol, belonging to the Structural formula III.
F-31: Brilliant green pigment (Shanghai J&K Chemical Technology Co., Ltd.).
F-32: Leucocrystal violet (Shanghai J&K Chemical Technology Co., Ltd.).
F-33: P-methyl benzenesulfonamide (Shanghai TCI Chemicals).

A sample preparation method (including film lamination, exposure, development, copper plating, tin plating), a sample evaluation method, and an evaluation result of Embodiments 28-46 and Comparative examples 9-11 were described below.

(1) Sample preparation method
[Film lamination]

A copper surface of a copper clad laminate was polished by using a grinding machine, and washing and drying were performed to obtain a bright and fresh copper surface. A pressure roller temperature of a laminator was set to 110° C., a convey speed was 1.5m/min, after a PE protective film on the surface of the photosensitive dry film obtained in the above embodiments and the comparative examples was removed, and the photosensitive dry film was thermally laminated to the copper clad laminate under a standard pressure, so as to obtain a laminated sample.

### [Exposure]

The laminated sample was allowed to stand for over 15 min, exposure was performed by using a Japan Adtec exposure machine, a model was IP-6, exposure was performed by using a LDI exposure machine with a wavelength being 405 nm, a photosensitivity test was performed by using a stouffer 41-order exposure ruler, and the number of exposure frames was controlled between 17 and 20.

### [Development]

The exposed sample was allowed to stand for over 15 min, a development temperature was 30° C., a pressure was 1.2 Kg/cm², a development solution was 1% wt sodium carbonate solution, a development time was 1.5-2.0 times of a minimum development time, and washing and drying were performed after development. A minimum time required for completely dissolving a resist layer on an unexposed portion was used as the minimum development time.

[Pattern plating]

A plating solution selected copper sulfate and a stannous sulfate system from Zhengtianwei, copper was plated first and then tin was plated, and details were as follows: acid degreasing (10% concentration, 10 min, 40° C.)→washing by water for 2 min→microetching for 1 min (60 g/L sodium persulfate+20 ml/L concentrated sulfuric acid)→washing by water for 1 min→acid leaching for 1 min (10% sulfuric acid solution)→copper plating (current density 2ASD, temperature 22-27° C., time 60 min)→washing by water for 1 min→acid leaching for 1 min (10% sulfuric acid solution)→tin plating (current density 1ASD, temperature 20-25° C., time 10 min).

### [Etching]

A developed copper plate was etched, an etching solution was copper chloride, an etching speed was 1.0 m/min, an etching temperature was 48° C., a spray pressure was 1.5 bar, a specific weight was 1.3 g/mL, acidity was 2 mol/L, with 140 g/L of copper ions, and a model of an etching machine was Dongguan Universe GL181946.

[Film removal]

A film removal solution was NaOH with a concentration being 3.0 wt %, a temperature was 50° C., a pressure was 1.2 Kg/cm², a film removal time was 1.5-2.0 times of a minimum film removal time, and washing and drying were performed after film removal.

Evaluation method
[Evaluation of photosensibility]

The laminated sample was allowed to stand for over 15 min, exposure was performed by using an Adtec IP-6 405 nm LDI exposure machine, a photosensibility test was performed by using a stouffer 41-order exposure ruler, after exposure, a 1% wt sodium carbonate solution was sprayed at 30° C., a development time is 2.0 times of the minimum development time, such that the unexposed portion was removed. After these operations, a cured film formed by solids of a photosensitive resin composition was formed a copper surface of a substrate. The photosensibility of a photosensitive resin composition was evaluated by an exposure amount (mJ/cm²) at which the number of residual segments of a stage-type exposure meter obtained as the cured film became 18 frames. If the numerical value was smaller, it indicated that the photosensibility was better.

Determination basis: o: 10-30 mJ/cm²
Δ: 30-50 mJ/cm²
x: >50 mJ/cm².
   [Evaluation of resolution ratio]

Exposure was performed by using a mask having a routing pattern with widths of an exposed portion and the unexposed portion being 1:1, development was performed with 2 times of the minimum development time, then a minimum mask width at which a cured resist line was normally formed was used as the value of the resolution ratio, a two-dimensional imager or a Scanning Electron Microscope (SEM) was used for observation, and if a number read was smaller, it indicated that the resolution ratio was more excellent.

[Evaluation of adhesion]

By laminating the photosensitive dry film resist on the copper plate through a hot pressed film, exposure was performed by using a mask having a routing pattern with widths of an exposed portion and the unexposed portion being n:400, corresponding sensitivity was 18 frames, development was performed with 2 times of the minimum development time, then a magnifier was used for observation, the minimum mask width at which the intact cured resist line was formed was used as the value of adhesion, and if the number read was smaller, it indicated that the adhesion is more excellent.

[Evaluation of electroplating resistance]

After film lamination, exposure, development, pattern plating, and film removal, an SEM was used for testing to observe whether a cementation phenomenon occurred.

Determination basis:
o: no cementation phenomenon.
Δ: slight cementation phenomenon.
x: serious cementation phenomenon.
   [Evaluation of electroplating pollution]

The exposed dry film resist sample (the number of exposure frames was 20) was dissolved in a copper sulfate plating solution at a proportion of 0.8 m²/L, soaking was performed for 24 h at room temperature, and then the dry film resist was filtered, so as to obtain a sample to be tested. A high-temperature catalytic combustion oxidation method was used, Total Organic Carbon (TOC) content of a plating solution sample to be tested was tested, and a plating solution sample not added with a resist sample was used as a blank sample. If a value of the obtained TOC is greater, it indicated that plating solution pollution was larger.

Determination basis:
o: after deduction of the blank, TOC value<500 ppm.
Δ: after deduction of the blank, 500 ppm>TOC value<1000 ppm.
x: after deduction of the blank, TOC value>1000 ppm.
   [Side shape evaluation]

After a PE film of the manufactured photosensitive dry film resist was removed, dry films were laminated on the copper plate by using a heated pressure roller. Here, exposure was performed by using the mask having the routing pattern with widths of the exposed portion and the unexposed portion being n:400, development was performed with 2.0 times of the minimum development time, then a dry film image was obtained, and the SEM was used to take a side view of a dry film with a line width being 25 um by magnifying 1000 times.

Determination basis:
o: Head sections of the dry films were rectangular.
Δ: Some head sections of the dry films were in an inverted trapezoidal shape.
x: The head sections of the dry films were shown to be heavily inverted trapezoidal shaped or the bottom was hollowed out, or an obvious crack phenomenon occurred in a sidewall.
(3) Evaluation results for performance such as photosensibility, resolution ratio, adhesion, electroplating resistance, electroplating pollution, and the like were shown in Table 11 and Table 12.

TABLE 11

| Evaluation | Embodiment | | | | | | | | | | |
|---|---|---|---|---|---|---|---|---|---|---|---|
| method | 28 | 29 | 30 | 31 | 32 | 33 | 34 | 35 | 36 | 37 | 38 |
| 405 nm | 16 | 17 | 13 | 16 | 16 | 10 | 20 | 16 | 16 | 13 | 27 |
| photosensibility | | | | | | | | | | | |
| mJ/cm² | | | | | | | | | | | |
| Resolution | 22.5 | 22.5 | 25 | 22.5 | 22.5 | 25 | 25 | 22.5 | 27.5 | 25 | 25 |
| ratio/um | | | | | | | | | | | |
| Adhesion/um | 25 | 25 | 25 | 25 | 25 | 25 | 27.5 | 25 | 25 | 27.5 | 27.5 |
| Electroplating | ∘ | ∘ | ∘ | ∘ | ∘ | ∘ | ∘ | Δ | ∘ | ∘ | ∘ |
| resistance | | | | | | | | | | | |
| Electroplating | ∘ | ∘ | ∘ | ∘ | ∘ | ∘ | ∘ | ∘ | ∘ | ∘ | ∘ |
| pollution | | | | | | | | | | | |
| Side shape | ∘ | ∘ | ∘ | ∘ | ∘ | ∘ | ∘ | ∘ | ∘ | ∘ | Δ |

TABLE 12

| Evaluation | Embodiment | | | | | | | | Comparative example | | |
|---|---|---|---|---|---|---|---|---|---|---|---|
| method | 39 | 40 | 41 | 42 | 43 | 44 | 45 | 46 | 9 | 10 | 11 |
| 405 nm | 17 | 10 | 15 | 17 | 17 | 50 | 15 | 120 | 15 | 18 | 12 |
| photosensibility | | | | | | | | | | | |
| mJ/cm² | | | | | | | | | | | |
| Resolution | 22.5 | 30 | 20 | 25 | 35 | 30 | 25 | 30 | 27 | 25 | 22.5 |
| ratio/um | | | | | | | | | | | |
| Adhesion/um | 25 | 30 | 22.5 | 27.5 | 35 | 30 | 27.5 | 30 | 30 | 25 | 25 |
| Electroplating | Δ | Δ | Δ | Δ | Δ | Δ | Δ | ∘ | × | × | × |
| resistance | | | | | | | | | | | |
| Electroplating | ∘ | ∘ | ∘ | ∘ | ∘ | ∘ | × | ∘ | × | ∘ | ∘ |
| pollution | | | | | | | | | | | |
| Side shape | ∘ | Δ | ∘ | ∘ | Δ | Δ | ∘ | Δ | Δ | Δ | Δ |

By comparing the embodiments with the comparative examples, it might be found that the dry film resists that had good key performance in terms of photosensibility, resolution ratio, adhesion performance, electroplating resistance, and electroplating pollution were all obtained in the embodiments.

In Embodiment 40, a ratio of the sensitizer to the complex exceeded a preferred ratio range, and the dry film resist, although highly photosensitive, corresponds to poor analysis, adhesion, and electroplating resistance. In Embodiment 43, the proportion of a PO chain segment in a light-curing monomer exceeded a preferred ratio range, the analysis, adhesion and side shape of the dry film resist were poor. In Embodiment 44, the pyrazoline sensitizer reported in Patent CN104111583 by Hitachi Chemical Industry Co., Ltd. was used, and the photosensibility, analysis, adhesion and side shape of the obtained dry film resist were poor. In Embodiment 45, the initiator system used was an initiator system commonly used in a high-sensitivity LDI resist in a current stage, but had poor electroplating resistance and electroplating pollution, and thus was not suitable for an electroplating process. Analysis reasons: on the one hand, since the molecular weight of an acridine initiator was relatively small, a use amount was relatively large, easily causing initiator fragments in the exposed dry film resist to penetrate into the plating solution, resulting in plating solution pollution; and on the other hand, for such initiator, the bottom side of the dry film was slightly uneven after exposure, resulting in a slight cementation phenomenon. In Embodiment 46, the initiator system used was an initiator system commonly used in an electroplated dry film in the current stage, and had poor photosensitivity to an LDI 405 nm laser light source, however, due to the addition of a complexant, the obtained dry film resist had excellent electroplating resistance.

In Comparative examples 9 and 10, no complexes were added, thus the electroplating resistance of the obtained dry film resist were relatively poor. In Comparative example 11, the light-curing monomer did not contain a hydrophobic PO chain segment and only contained a hydrophilic EO segment, and although the obtained dry film resist was excellent in terms of photosensibility, resolution ratio, and adhesion, the electroplating resistance was poor, thereby occurring a slight cementation phenomenon.

The above are only the preferred embodiments of the present disclosure and are not intended to limit the present disclosure. For those skilled in the art, the present disclosure may have various modifications and variations. Any modifications, equivalent replacements, improvements and the like made within the spirit and principle of the present disclosure all fall within the scope of protection of the present disclosure.

1. A dry film resist, comprising an alkali-soluble resin (A), a photopolymerization monomer (B), a photoinitiator (C), and a sensitizer (D), wherein the sensitizer (D) comprises a compound containing a pyrazoline structure.

2. The dry film resist according to claim 1, wherein the sensitizer (D) comprises a compound containing an anthracene-substituted and/or triarylamine-substituted pyrazoline structure;
preferably, the sensitizer (D) comprises any one or more of the following compounds shown in structural formulas D1, D2, D3, D4, and D5, 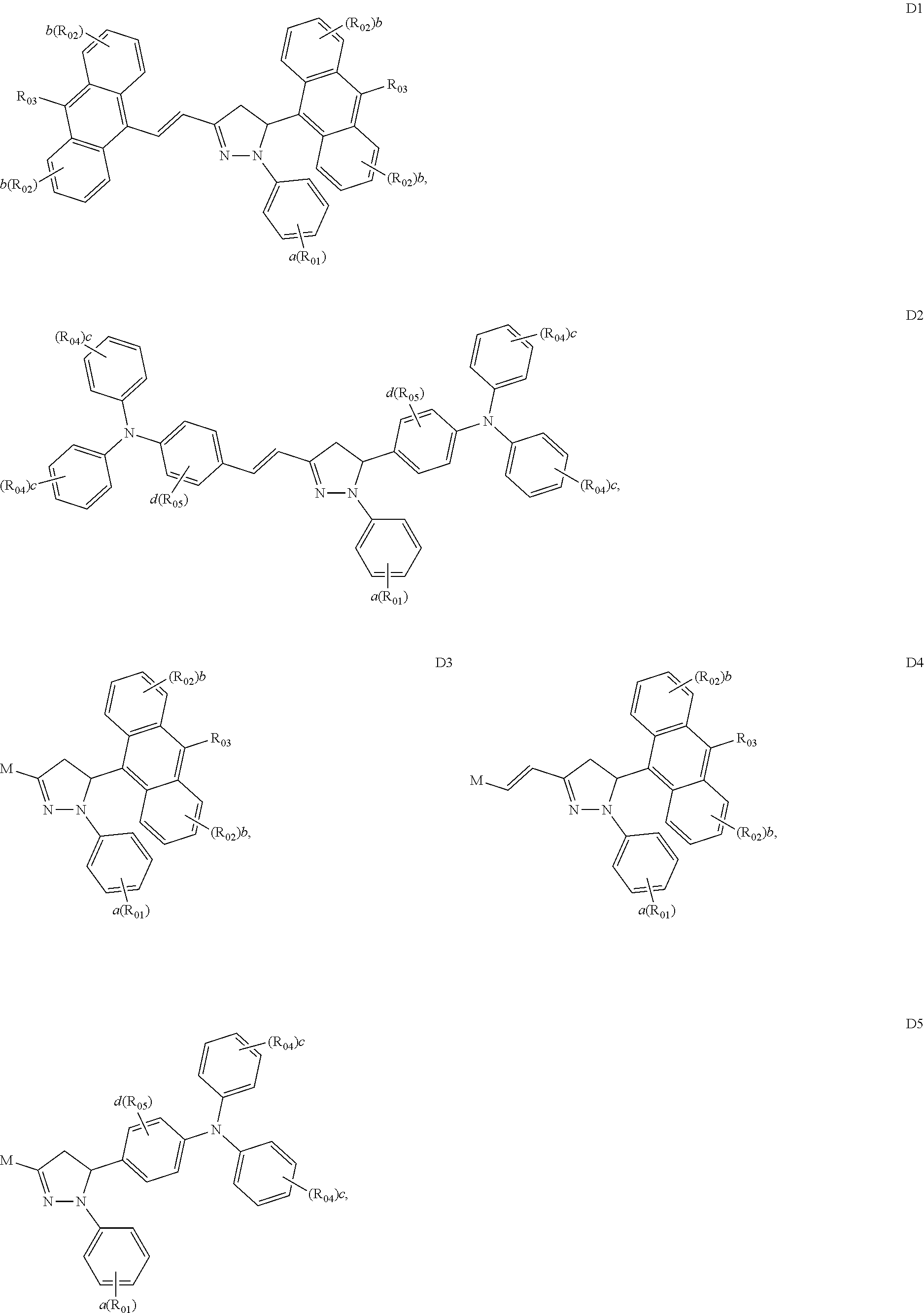
wherein R₀₁, R₀₂, R₀₃, R₀₄, R₀₅ are independently any one or more of hydrogen, halogen, nitro, C₁-C₈ alkyl, and C₁-C₄ alkoxy, respectively;
a represents any one of integers among 0-5, b represents any one of integers among 0-4, c represents any one of integers among 0-5, d represents any one of integers among 0-4, and when a is greater than or equal to 2, a plurality of existing R₀₁s are able to be the same or different, respectively; when b is greater than or equal to 2, a plurality of existing R₀₂s are able to be the same or different, respectively; when c is greater than or equal to 2, a plurality of existing R₀₄s are able to be the same or different, respectively; when d is greater than or equal to 2, a plurality of existing R₀₅s are able to be the same or different, respectively;
Ms are independently any one of phenyl, biphenyl, and a condensed ring group respectively, or an electron-rich heterocyclic or fused-heterocyclic group;
preferably, the M is phenyl, biphenyl or polycyclic group with any one or more of C₁-C₄ alkoxy, amino, and alkyl, or is a heterocyclic or fused-heterocyclic group with furan, thiophene, indole, thiazole, benzofuran, benzothiazole, or fluorene;
more preferably, the sensitizer comprises compounds having the following structures, 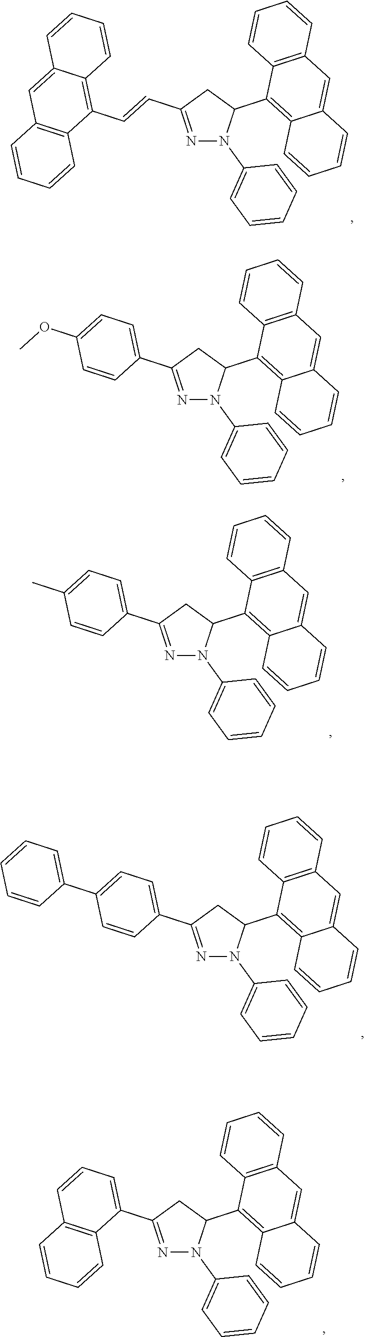 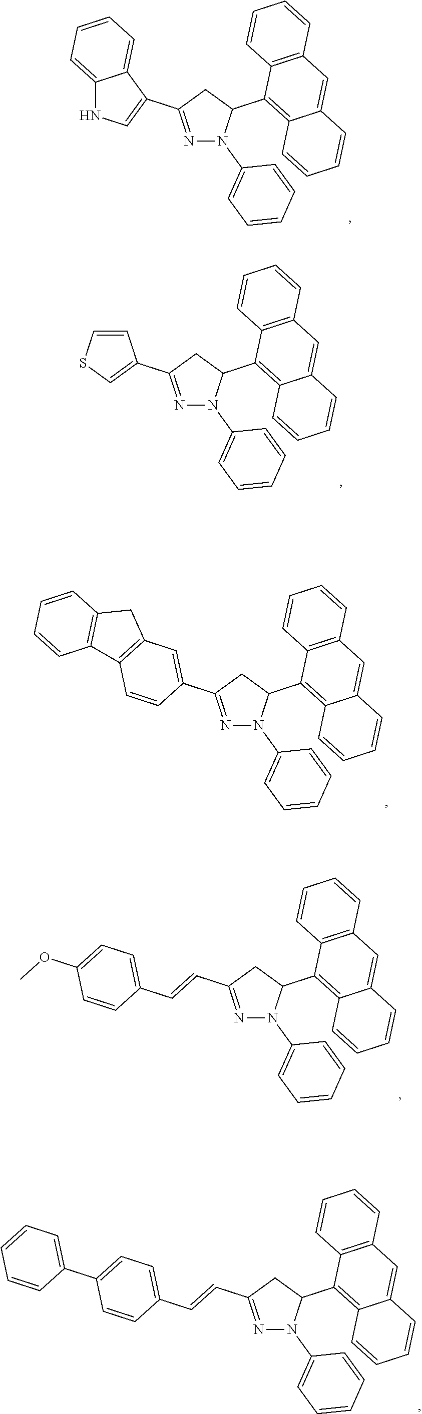 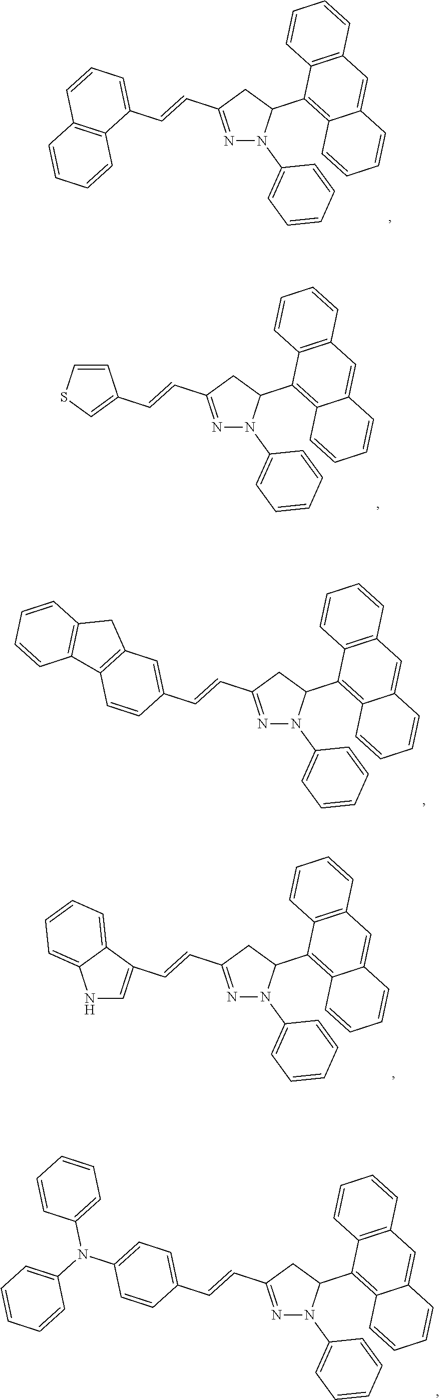 [SMILES: C(=C\c1cccc2ccccc12)/C1=NN(c2ccccc2)C(c2c3ccccc3cc3ccccc23)C1 ; C(=C\c1ccsc1)/C1=NN(c2ccccc2)C(c2c3ccccc3cc3ccccc23)C1 ; C(=C\c1ccc2c(c1)Cc1ccccc1-2)/C1=NN(c2ccccc2)C(c2c3ccccc3cc3ccccc23)C1 ; C(=C\c1c[nH]c2ccccc12)/C1=NN(c2ccccc2)C(c2c3ccccc3cc3ccccc23)C1 ; C(=C\c1ccc(N(c2ccccc2)c2ccccc2)cc1)/C1=NN(c2ccccc2)C(c2c3ccccc3cc3ccccc23)C1] 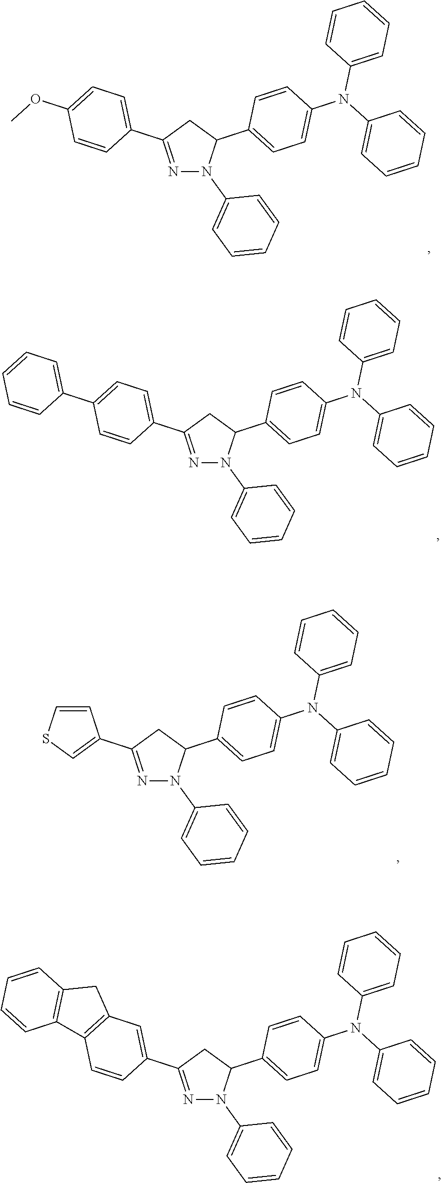 [SMILES: COc1ccc(C2=NN(c3ccccc3)C(c3ccc(N(c4ccccc4)c4ccccc4)cc3)C2)cc1 ; c1ccc(-c2ccc(C3=NN(c4ccccc4)C(c4ccc(N(c5ccccc5)c5ccccc5)cc4)C3)cc2)cc1 ; c1ccc(N(c2ccccc2)c2ccc(C3CC(c4ccsc4)=NN3c3ccccc3)cc2)cc1 ; c1ccc(N(c2ccccc2)c2ccc(C3CC(c4ccc5c(c4)Cc4ccccc4-5)=NN3c3ccccc3)cc2)cc1] 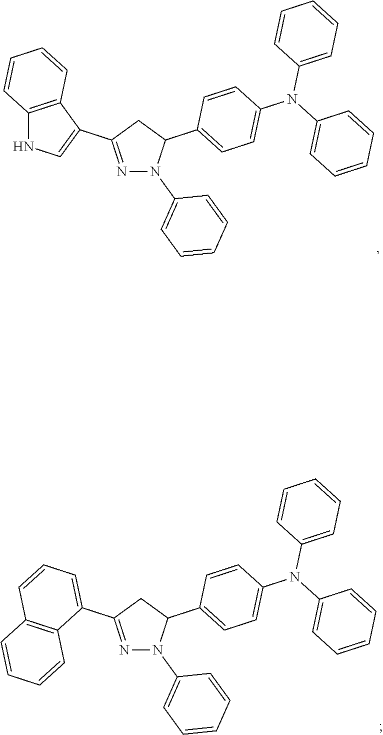 [SMILES: c1ccc(N(c2ccccc2)c2ccc(C3CC(c4c[nH]c5ccccc45)=NN3c3ccccc3)cc2)cc1 ; c1ccc(N(c2ccccc2)c2ccc(C3CC(c4cccc5ccccc45)=NN3c3ccccc3)cc2)cc1]
and optionally, the benzene ring, biphenyl ring, fused ring, or heterocyclic ring in the above structures contains a substituent, the substituent is any one or more of halogen, C₁-C₅ alkyl, and C₁-C₄ alkoxy, and preferably, the substituent is located in a para-position.

3. The dry film resist according to claim 1, wherein the sensitizer (D) comprises any one or more of the following compounds shown in a Structural formula I or Structural formula II, 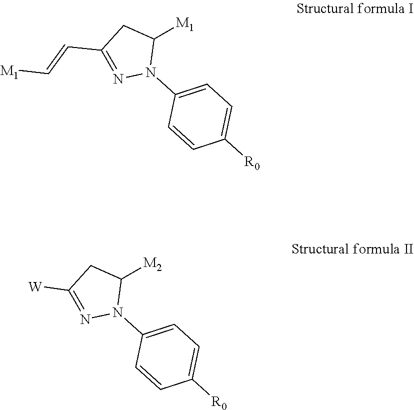
wherein R₀s are independently hydrogen, halogen, C₁-C₅ alkyl, and C₁-C₄ alkoxy, respectively; modified groups M₁s in the Structural formula I are independently a biphenyl group, a condensed ring group or electron-rich heterocycle, fused electron-rich heterocycle, or a benzene ring with any one or more of C₁-C₄ alkoxy or amino substituents; a modified group M₂ in the Structural formula II is selected from biphenyl, a condensed ring group or electron-rich heterocycle, fused electron-rich heterocycle, or a benzene ring with any one or more of C₁-C₄ alkoxy, C₁-C₃ alkyl and amino substituents; and a modified group W is any one of a benzene ring and a fluorene ring, or is a benzene ring, a biphenyl ring, or a fluorene ring with any one or more of halogen, C₁-C₅ alkyl, and C₁-C₄ alkoxy substituents;
preferably, the modified groups M₁s are independently condensed ring groups with any one or more of C₁-C₄ alkoxy, amino, and alkyl respectively, or are any one of furan, thiophene, indole, thiazole, benzofuran, benzothiazole, indene, anthracene, acridine, and aromatic amine, and
preferably, a specific structural formula of the modified group M₁ and/or M₂ comprises any one of 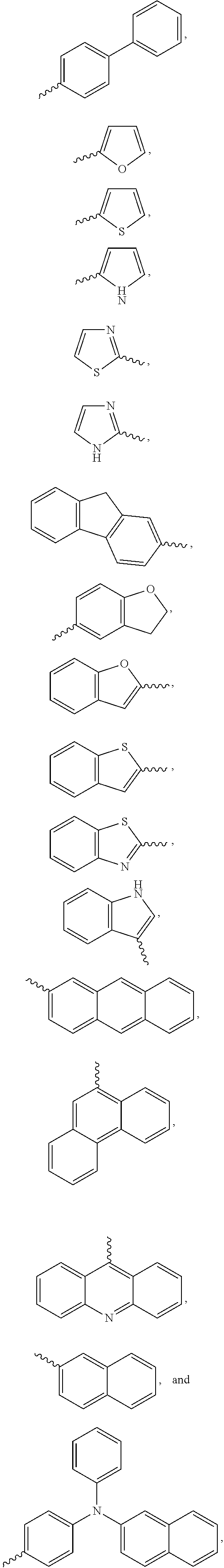
wherein [symbol] is a binding position of a group; and optionally, the benzene ring, biphenyl ring, fused ring, or heterocyclic ring on the specific structural formula of the modified group M₁ and/or M₂ contains any one or more of halogen, C₁-C₅ alkyl, and C₁-C₄ alkoxy substituents, and preferably, the substituent is located in a para-position.

4. The dry film resist according to claim 1,
comprising: by weight, 45-65 parts of the alkali-soluble resin (A), 30-50 parts of the photopolymerization monomer (B), 1.0-5.0 parts of the photoinitiator (C), and 0.01-0.5 parts of the sensitizer (D);
preferably, the dry film resist comprises, by weight, 45-65 parts of the alkali-soluble resin (A), 30-50 parts of the photopolymerization monomer (B), 2.0-5.0 parts of the photoinitiator (C), and 0.01-0.5 parts of the sensitizer (D); and
preferably, the content of the sensitizer is 0.01 wt %-0.5 wt % of a total weight of the dry film resist.

5. The dry film resist according to claim 1,
wherein the photoinitiator (C) comprises the following compound shown in a structural formula C₁, 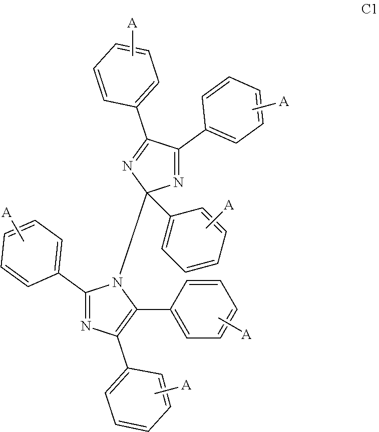
wherein substituents A on the benzene rings are independently any one or more of hydrogen, methoxy, and halogen atoms, respectively; and
preferably, the photoinitiator (C) comprises one or more selected from a 2-(o-chlorophenyl)-4,5-diphenylimidazole dimer, a 2-(o-chlorophenyl)-4,5-di(methoxyphenyl)imidazole dimer, a 2-(o-fluorophenyl)-4,5-diphenylimidazole dimer, a 2-(o-methoxyphenyl)-4,5-diphenylimidazole dimer, a 2-(p-methoxyphenyl)-4,5-diphenylimidazole dimer, and
2,2′,4-tris(2-chlorophenyl)-5-(3,4-dimethoxyphenyl)-4′,5′-diphenyl-1,1′-biimi dazole.

6. (canceled)

7. The dry film resist according to claim 2, wherein the photopolymerization monomer (B) comprises any one or more of the following compounds shown in structural formulas B1, B2, and B3, 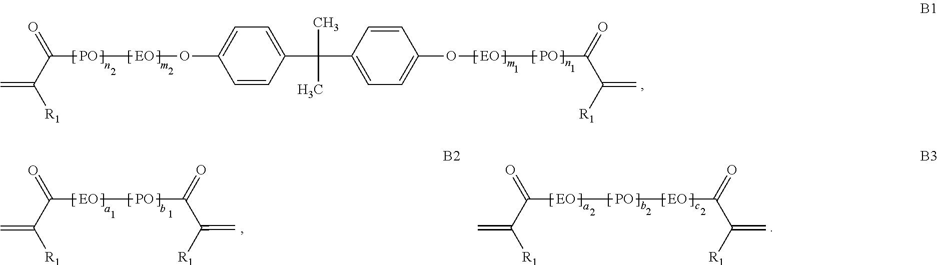
wherein R₁s are independently H or CH₃ respectively, EO represents the oxoethylidene, PO represents the oxypropylidene, and EO and PO repeating units are arranged in a random or segmented manner; m₁ and m₂ are respectively any one of integers among 1-20, n₁ and n₂ are respectively any one of integers among 0-20, m₁+m₂ is any one of integers among 2-20, and n₁+n₂ is any one of integers among 0-20; a₁ is any one of integers among 4-20, and b₁ is any one of integers among 0-20; a₂ is any one of integers among 3-20, b₂ is any one of integers among 0-20, and c₂ is any one of integers among 3-20;
preferably, a photopolymerization monomer having a structure shown in the structural formula B1 accounts for 40%-90% of a total amount of the photopolymerization monomer (B), further preferably accounts for 50%-80%, and is 20%-40% of a total weight of the photopolymerization monomer (B) and the alkali-soluble resin (A);
further preferably, the photopolymerization monomer (B) further comprises any one or more of structures shown in the structural formula B4, 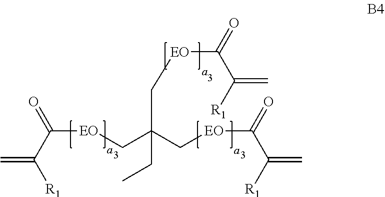
in the formula, R₁s are independently H or CH₃ respectively, and a₃s are independently any integers among 1-10 respectively; and
more preferably, the photopolymerization monomer (B) is selected from any one or more of lauryl acrylate, lauryl methacrylate, octadecyl acrylate, octadecyl methacrylate, nonylphenol acrylate, isobornyl methacrylate, tetrahydrofurfuryl acrylate, bisphenol A diacrylate, bisphenol A dimethacrylate, poly ethylene glycol diacrylate, poly propylene glycol diacrylate, poly ethylene glycol dimethacrylate, poly propylene glycol dimethacrylate, ethoxylated neopentyl glycol diacrylate, propoxylated neopentyl glycol diacrylate, trimethylolpropane triacrylate, trimethylol propane trimethacrylate, ethoxylated trimethylolpropane trimethacrylate, propoxylated trimethylolpropane trimethacrylate, ethoxylated trimethylolpropane triacrylate, propoxylated trimethylolpropane triacrylate, ethoxylated pentaerythritol triacrylate, propoxylated pentaerythritol triacrylate, ethoxylated pentaerythritol tetraacrylate, propoxylated pentaerythritol tetraacrylate, ethoxylated dipentaerythritol pentaacrylate, propoxylated dipentaerythritol pentaacrylate, propoxylated dipentaerythritol hexaacrylate and ethoxylated dipentaerythritol hexaacrylate.

8. The dry film resist according to claim 3, wherein the photopolymerization monomer (B) comprises any one or more of the following compounds shown in a structural formula B1, 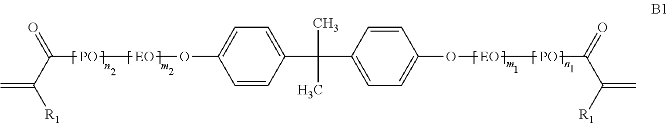
in the formula, R₁s are independently H or CH₃ respectively, m₁ and m₂ are respectively any one of integers among 1-20, n₁ and n₂ are respectively any one of integers among 0-20, m₁+m₂ is any one of integers among 2-20, n₁+n₂ is any one of integers among 0-20, EO represents the oxoethylidene, PO represents the oxypropylidene, and the EO and PO repeating units are arranged in a random or segmented manner;
preferably, a photopolymerization monomer having a structure shown in the structural formula B1 accounts for 20%-80% of a total amount of the photopolymerization monomer, further preferably accounts for 40%-80%, and is 10%-35% of a total weight of the photopolymerization monomer (B) and the alkali-soluble resin (A); and
further preferably, the photopolymerization monomer (B) further comprises any one or more selected from lauryl acrylate, lauryl methacrylate, octadecyl acrylate, octadecyl methacrylate, nonylphenol acrylate, isobornyl methacrylate, tetrahydrofurfuryl acrylate, bisphenol A diacrylate, bisphenol A dimethacrylate, poly ethylene glycol diacrylate, poly propylene glycol diacrylate, poly ethylene glycol dimethacrylate, poly propylene glycol dimethacrylate, ethoxylated neopentyl glycol diacrylate, propoxylated neopentyl glycol diacrylate, trimethylolpropane triacrylate, trimethylolpropane trimethacrylate, ethoxylated trimethylolpropane trimethacrylate, propoxylated trimethylolpropane trimethacrylate, ethoxylated trimethylolpropane triacrylate, propoxylated trimethylolpropane triacrylate, pentaerythritol triacrylate, pentaerythritol tetraacrylate, dipentaerythritol pentaacrylate, and dipentaerythritol hexaacrylate.

9. (canceled)

10. The dry film resist according to claim 1,
wherein the alkali-soluble resin (A) is formed by copolymerizing methacrylic acid, and/or acrylic acid, methacrylate and/or acrylate, and styrene or derivatives of the styrene, and the alkali-soluble resin (A) comprises any one or more of the following compound shown in a structural formula A1, 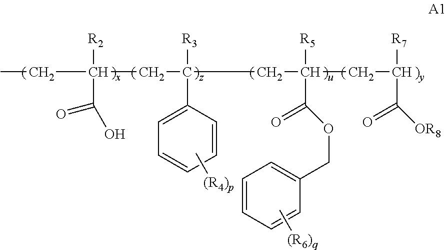
R₂, R₃, R₅, and R₇ are independently hydrogen or methyl respectively; R₄ and R₆ are independently any one or more of alkyl having 1-5 carbon atoms, alkoxy having 1-5 carbon atoms, hydroxyl or halogen atoms respectively; p and q independently represent any integer among 0-5 respectively; R8 is any one of straight chain, branched chain or cyclic alkyl having 1-18 carbon atoms; and x, y, z, and u respectively represent specific weights of various copolymerization components in the alkali-soluble resin, wherein x is 15-40 wt %, z is 0-50 wt %, u is 0-80 wt %, and y is 0-40 wt %; and
preferably, an acid value of the alkali-soluble resin (A) is 120-250 mg KOH/g, more preferably, a weight-average molecular weight is 30000-120000, a molecular weight distribution is 1.3-2.5, and further preferably, a polymerization conversion rate is greater than or equal to 97%.

11. The dry film resist according to claim 10, wherein, in the structural formula A1, x is 15-35 wt %, z is 0-50 wt %, u is 0-80 wt %, y is 0-25 wt %, and a value of z+u is 40 wt %-80 wt %;
preferably, a copolymerization unit of the alkali-soluble resin (A) is selected from one or more of methyl methacrylate, methyl acrylate, ethyl methacrylate, ethyl acrylate, propyl methacrylate, propyl acrylate, isopropyl methacrylate, isopropyl acrylate, butyl methacrylate, butyl acrylate, isobutyl methacrylate, isobutyl acrylate, methylheptyl methacrylate, methylheptyl acrylate, dodecyl methacrylate, dodecyl acrylate, stearyl methacrylate, stearyl acrylate, 2-hydroxyethyl methacrylate, 2-hydroxyethyl acrylate, 2-hydroxypropyl methacrylate, 2-hydroxypropyl acrylate, 4-hydroxybutyl methacrylate, 4-hydroxybutyl acrylate, glycidyl methacrylate, glycidyl acrylate, N,N-dimethyl ethyl acrylate, N,N-dimethylethyl methacrylate, N,N-diethylethyl acrylate, N,N-diethylethyl methacrylate, N,N-diethylpropyl acrylate, N,N-diethylpropyl methacrylate, N,N-dimethylbutyl acrylate, N,N-dimethylbutyl methacrylate, N,N-diethylbutyl methacrylate and N,N-diethylbutyl acrylate; and
preferably, the weight-average molecular weight of the alkali-soluble resin (A) is 30000-80000, and the molecular weight distribution is 1.3-2.5.

12. The dry film resist according to claim 10, wherein, in the structural formula A1, R₃ is hydrogen, x is 15-40 wt %, z is 0-40 wt %, u is 0, and y is 20-60 wt %;
preferably, a copolymerization unit of the alkali-soluble resin (A) is selected from alkyl methacrylate and/or alkyl acrylate and styrene and/or derivatives thereof; the alkyl methacrylate is selected from any one or more of methyl methacrylate, ethyl methacrylate, propyl methacrylate, isopropyl methacrylate, butyl methacrylate, isobutyl methacrylate, methylheptyl methacrylate, dodecyl methacrylate, stearyl methacrylate, 2-hydroxyethyl methacrylate, 2-hydroxypropyl methacrylate, 4-hydroxybutyl methacrylate, glycidyl methacrylate, N,N-dimethylethyl methacrylate, N,N-diethylethyl methacrylate, N,N-diethylpropyl methacrylate, N,N-dimethylbutyl methacrylate, and N,N-diethylbutyl methacrylate; the alkyl acrylate is selected from any one or more of methyl acrylate, ethyl acrylate, propyl acrylate, isopropyl acrylate, butyl acrylate, isobutyl acrylate, methylheptyl acrylate, dodecyl acrylate, stearyl acrylate, 2-hydroxyethyl acrylate, 2-hydroxypropyl acrylate, 4-hydroxybutyl acrylate, glycidyl acrylate, N,N-dimethylethyl acrylate, N,N-diethylethyl acrylate, N,N-diethylpropyl acrylate, N,N-dimethylbutyl acrylate, and N,N-diethylbutyl acrylate; the styrene derivatives are selected from one or more of α-methylstyrene, benzyl acrylate, and benzyl methacrylate;
further preferably, the content of the styrene in the copolymerization unit of the alkali-soluble resin (A) is 0-40 wt % of a total weight of the copolymerization unit; and
preferably, the weight-average molecular weight of the alkali-soluble resin (A) is 30000-80000, and the molecular weight distribution is 1.3-2.5.

13. (canceled)

14. The dry film resist according to claim 1, further comprising an additive, wherein the additive comprises any one or more of a free radical polymerization inhibitor, a color former, a coloring agent, a plasticizer, a photothermal stabilizer, an adhesion promoter, a leveling agent, and a defoaming agent, and preferably, the content of the additive is 0.5-5.0 parts by mass;
further preferably, the content of the free radical polymerization inhibitor is 0.001 wt %-0.005 wt % of the total weight of the dry film resist; and further preferably, the free radical polymerization inhibitor is selected from any one or more of 4-methoxyphenol, 4-ethyl-6-tert-butylphenol, nitroso phenylhydroxylamine aluminum salt, dimethyl-1,2-benzenediol, 3-methylcatechol, 4-methylcatechol, catechol, 2-ethylcatechol, 3-ethylcatechol, 4-ethylcatechol, 2-propylcatechol, 3-propylcatechol, 4-propylcatechol, 2-n-butylcatechol, 3-n-butylcatechol, 4-n-butylcatechol, 2-tert-butylcatechol, 3-tert-butylpyrocatechol, 4-tert-butylcatechol, 3,5-di-tert-butylcatechol, resorcinol, 2-methylresorcinol, 4-methylresorcinol, 5-orcinol, 2-ethylresorcinol, 4-ethylresorcinol, 2-propylresorcinol, 4-propylresorcinol, 2-n-butylresorcinol, 4-butylresorcinol, 2-tert-butylresorcinol, 4-tert-butylresorcinol, 1,4-hydroquinone, methylhydroquinone, ethylhydroquinone, propyl hydroquinone, tert-butylhydroquinone, 2,5-di-tert-butylhydroquinone, 2,6-di-tert-butyl-4-methylphenol, pyrogallol, 4-hydroxy-2,2,6,6-tetramethylpiperidine-N-oxyl, and 2,2-methylenebis(4-methyl-6-tert-butylphenol).

15. A photosensitive dry film, comprising a dry film resist layer, and a support layer and a protective layer located on two sides of the dry film resist layer, wherein the dry film resist layer contains the dry film resist according to claim 1.

16. A dry film resist, comprising an alkali-soluble resin (A), a photopolymerization monomer (B), a photoinitiator (C), and a copper complex (E), wherein a compound that forms a complex with copper in the copper complex (E) comprises a nitrogen-containing heterocycle compound, the photopolymerization monomer (B) contains an EO chain segment and a PO chain segment, the EO chain segment represents oxoethylidene, and the PO chain segment represents oxypropylidene.

17. The dry film resist according to claim 16, wherein the compound that forms the complex with copper in the copper complex (E) contains nitrogen heterocycle and sulfydryl at the same time; preferably, the compound that forms the complex with copper in the copper complex (E) has any one or more of the following structures shown in a Structural formula III or Structural formula IV, 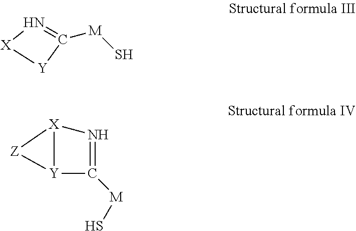
in the Structural formula III or Structural formula IV, X is 1-3 carbon atoms, or 1-2 nitrogen atoms, or one carbon atom and one nitrogen atom, and the carbon atoms and/or nitrogen atoms are linked by single bonds or double bonds; Y is selected from one or more of an oxygen atom, a sulfur atom, a carbon atom, and a nitrogen atom, and the hydrogen atom on a ring formed by the X, the Y and —C═NH— is able to be substituted by any one or more of carboxyl, amino, C₁-C₁₂ alkyl, C₁-C₁₂ alkoxy, C₆-C₁₂ aryl, and hydrazinyl; M is selected from a single bond, C₁-C₁₂ alkyl, a C₁-C₁₂ ester group, or a C₂-C₁₂ ether group; in the Structural formula IV, a ring formed by X, Y, and Z is a benzene ring or a heterocyclic ring, and the benzene ring and the heterocyclic ring are able to carry any one or more of C₁-C₆ alkyl, C₁-C₆ alkoxy, amino, carboxylic acid, nitro, and halogen; and
further preferably, the compound that forms the complex with copper in the copper complex (E) is selected from any one or more of mercaptopyrimidine, 4,6-diamino-2-mercaptopyrimidine, mercaptoimidazole, mercaptobenzimidazole, 2-mercapto-5-carboxybenzimidazole, 2-mercapto-5-nitrobenzimidazole, 5-amino-2-benzimidazolethiol, 2-mercapto-1H-benzo[d]iMidazole-4-carboxylic acid, mercaptobenzothiazole, 3-mercaptoindole, 1,3,5-tris(thioethyl)-1,3,5-triazine-2,4,6-trione, mercaptopurine, 6-thioguanine, trithiocyanuric acid, 2,6-dithiopurine, 4-thiouracil, 2-mercaptobenzoxazole, 4,6-diamino-2,6-mercaptopyrimidine, 4-thiouracil, 2-mercapto-4-hydroxy-5,6-diaminopyrimidine, 4,6-dimethyl-2-mercaptopyrimidine, dithiouracil, 2-mercaptopyrazine, 3,6-dimercaptopyridazine, 2-mercaptoimidazole, 2-mercaptothiazole, 8-mercaptoadenine, 4-thiouracil, mercaptotriazole, a 3-mercapto-1,2,4-triazole disulfide, 3-amino-5-mercapto-1,2,4-triazole, 4-methyl-1,2,4-triazole-3-thiol, 3-amino-5-mercapto-1,2,4-triazole, 3-mercapto-1,2,4-triazole, a 6,7-dihydro-6-mercapto-5H-pyrazolo[1,2-A][1,2,4]triazole chloride, 4-amino-3-hydrazino-1,2,4-triazol-5-thiol, 5-mercapto-1-methyltetrazole, 2-(5-mercaptotetrazole-1-yl)ethanol, 1-[2-(dimethylamino)ethyl]-1H-tetrazole-5-thiol, 1-phenyltetrazole-5-thiol, 1,2-dihydro-1-(4-methoxyphenyl)-5H-tetrazole-5-thione, 1-ethyl-1H-1,2,3,4-tetrazole-5-thiol, 5-mercapto-1H-tetrazole-1-acetic acid, 5-mercapto-1H-tetrazole-1-methane sulphonic acid, N-[3-(5-mercapto-1H-1,2,3,4-tetraazol-1-yl)phenyl]acetamide, 1-(4-hydroxyphenyl)-2H-tetrazole-5-thione, 1-(4-ethoxyphenyl)-5-mercapto-1H-tetrazole, 1-(4-carboxyphenyl)-5-mercapto-1H-tetrazole, and 4-amino-2-mercaptopyrimidine.

18. The dry film resist according to claim 16, comprising: by weight, 40-65 parts of the alkali-soluble resin (A), 35-60 parts of the photopolymerization monomer (B), 2.0-4.5 parts of the photoinitiator (C), and 0.01-0.5 parts of the copper complex (E);
the dry film resist further comprising an additive, wherein the additive comprises any one or more of a color former, a coloring agent, a plasticizer, a photothermal stabilizer, an adhesion promoter, a leveling agent, a polymerization inhibitor, and a defoaming agent, and preferably, the content of the additive is 0.5-5.0 parts by weight;
the dry film resist further comprising 0.01-0.5 parts of a sensitizer (D): preferably, the sensitizer (D) comprises any one or more of the following compounds shown in a Structural formula I or Structural formula II, 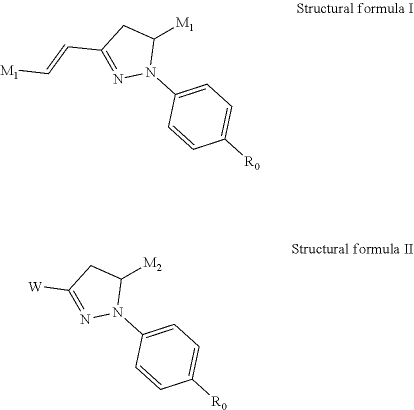
Wherein R₀s are independently hydrogen, halogen, C₁-C₅ alkyl, and C₁-C₄ alkoxy, respectively;
modified groups M₁s in the Structural formula I are independently biphenyl, a condensed ring group or electron-rich heterocycle, fused electron-rich heterocycle, or a benzene ring with C₁-C₄ alkoxy or amino;
a modified group M₂ in the Structural formula II is selected from any one of biphenyl, a condensed ring group or electron-rich heterocycle, fuse electron-rich heterocycle, or a benzene ring with C₁-C₄ alkoxy or amino, or phenyl with C₁-C₄ alkoxy substituents; and a modified group W is any one of a benzene ring and a fluorene ring, or is a benzene ring, a biphenyl ring, or a fluorene ring with any one or more of halogen, C₁-C₅ alkyl, and C₁-C₄ alkoxy substituents;
preferably, the modified groups M₁s are independently condensed ring groups with any one or more of C₁-C₄ alkoxy, amino, and alkyl respectively, or are any one of furan, thiophene, indole, thiazole, benzofuran, benzothiazole, indene, anthracene, acridine, and aromatic amine, and further preferably, a specific structural formula of the modified group M₁ and/or M₂ comprises; 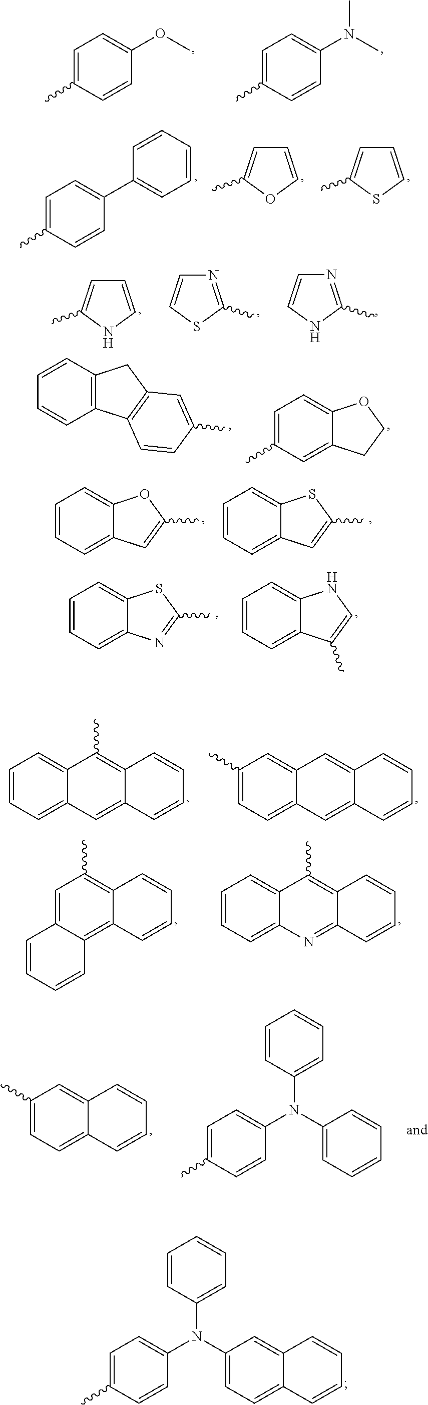
wherein [symbol] is a binding position of a group,
optionally, the benzene ring, biphenyl ring, fused ring, or heterocyclic ring in the specific structural formula of the modified group M₁ and/or M₂ contains any one or more of substituents in halogen, C₁-C₅ alkyl, and C₁-C₄ alkoxy, and preferably, the substituent is located in a para-position.

19. (canceled)

20. The dry film resist according to claim 16, wherein the photoinitiator (C) comprises the following compound shown in a Structural formula C₁, 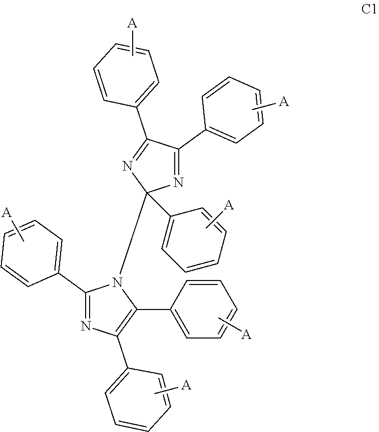
wherein substituents A on the benzene rings are independently hydrogen, methoxy, and halogen atoms;
preferably, the photoinitiator (C) comprises any one or more selected from a 2-(-chlorophenyl)-4,5-diphenylimidazole dimer, a 2-(o-chlorophenyl)-4,5-di(methoxyphenyl)imidazole dimer, a 2-(o-fluorophenyl)-4,5-diphenylimidazole dimer, a 2-(o-methoxyphenyl)-4,5-diphenylimidazole dimer, a 2-(p-methoxyphenyl)-4,5-diphenylimidazole dimer, and 2,2′,4-tris(2-chlorophenyl)-5-(3,4-dimethoxyphenyl)-4′,5′-diphenyl-1,1′-biimi dazole; and
optionally, the photoinitiator (C) further comprises any one or more selected from thioxanthone, benzoin phenyl ether, benzophenone, benzoin methyl ether, N,N′-tetramethyl-4,4′-diaminobenzophenone, N,N′-tetraethyl-4,4′-diaminobenzophenone, 4-methoxy-4′-dimethylamino diphenyl ketone, 2-benzyl-2-dimethylamino-1-(4-morpholinephenyl)-butanone, 2-ethyl anthraquinone, phenanthrenequinone, 2-tert-butylanthraquinone, octamethylanthraquinone, 1,2-benzanthraquinone, 2,3-benzoanthraquinone, 2,3-diphenylanthraquinone, 1-chloro anthraquinone, 2-methyl anthraquinone, 1,4-naphthoquinone, 9,10-phenanthrenequinone, 2,3-dimethylanthraquinone, benzoin methyl ether, benzoin ethyl ether, benzoin phenyl ether, 2,2-dimethoxy-2-phenylacetophenone, 9-phenylacridine, 1,7-bis(9,9′-acridinyl)heptane, anilinoacetic acid, a coumarin-based compound, and an oxazole-based compound.

21. The dry film resist according to claim 16, wherein the photopolymerization monomer comprises any one or more of the following compounds shown in structural formulas B1, B5, B6, and B7, 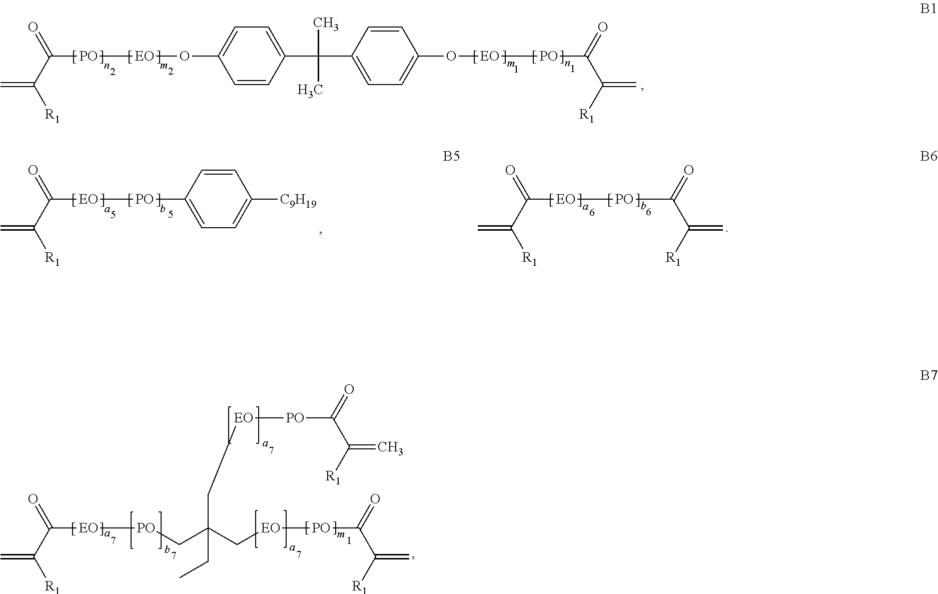
in the formula, R₁s are independently H or CH₃ respectively, and the EO chain segment and PO chain segment repeating units are arranged in a random or segmented manner; in the structural formula B1, m₁ and m₂ are respectively any one of integers among 0-30, n₁ and n₂ are respectively any one of integers among 0-20, m₁+m₂ is any one of integers among 0-30, and n₁+n₂ is any one of integers among 0-20; in the structural formula B5, a₅ is any integer among 0-30, and b₅ is any integer among 0-20; in the structural formula B6, a₆ is any integer among 0-30, b₆ is any integer among 0-20; in the structural formula B7, a₇ is any integer among 0-20, and b₇ is any integer among 0-20;
preferably, a molar quantity of the PO chain segment is 15%-60% of a total molar quantity of the EO chain segment and the PO chain segment in the photopolymerization monomer; and
further preferably, the photopolymerization monomer (B) further comprises any one or more selected from lauryl acrylate, lauryl methacrylate, octadecyl acrylate, octadecyl methacrylate, nonylphenol acrylate, isobornyl methacrylate, tetrahydrofurfuryl acrylate, bisphenol A diacrylate, bisphenol A dimethacrylate, poly ethylene glycol diacrylate, poly propylene glycol diacrylate, poly ethylene glycol dimethacrylate, poly propylene glycol dimethacrylate, ethoxylated neopentyl glycol diacrylate, propoxylated neopentyl glycol diacrylate, trimethylolpropane triacrylate, trimethylolpropane trimethacrylate, ethoxylated trimethylolpropane trimethacrylate, propoxylated trimethylolpropane trimethacrylate, ethoxylated trimethylolpropane triacrylate, propoxylated trimethylolpropane triacrylate, pentaerythritol triacrylate, pentaerythritol tetraacrylate, dipentaerythritol pentaacrylate, dipentaerythritol hexaacrylate, and polyurethane acrylate.

22. The dry film resist according to any one of claim 16, wherein the alkali-soluble resin (A) is formed by copolymerizing methacrylic acid, and/or acrylic acid, methacrylate and/or acrylate, and styrene or derivatives of the styrene, and the alkali-soluble resin comprises any one or more of the following structures shown in a structural formula A2, 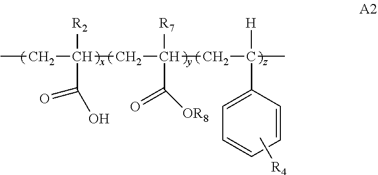
in the formula, R₂ and R₇ are independently hydrogen or methyl respectively; R₈ is selected from any one of C₁-C₁₈ straight chain alkyl, C₃-C₁₈ branched chain alkyl, benzyl, and C₁-C₁₈ straight chain alkyl or C₃-C₁₈ branched chain alkyl containing hydroxyl and/or amino substituents; R₄ is any one of C₁-C₃ alkyl, C₁-C₃ alkoxy, amino, and halogen atoms; the number of substituents on a benzene ring of the structural formula A2V is 0-5; x, y, and z respectively represent specific weights of various copolymerization components in the alkali-soluble resin, wherein x is 15-40 wt %, y is 20-70 wt %, and z is 0-40 wt %; preferably, the methacrylate is selected from any one or more of methyl methacrylate, ethyl methacrylate, propyl methacrylate, isopropyl methacrylate, butyl methacrylate, isobutyl methacrylate, methylheptyl methacrylate, dodecyl methacrylate, stearyl methacrylate, 2-hydroxyethyl methacrylate, 2-hydroxypropyl methacrylate, 4-hydroxybutyl methacrylate, glycidyl methacrylate, N,N-dimethylethyl methacrylate, N,N-diethylethyl methacrylate, N,N-diethylpropyl methacrylate, N,N-dimethylbutyl methacrylate, and N,N-diethylbutyl methacrylate; the alkyl acrylate is selected from any one or more of methyl acrylate, ethyl acrylate, propyl acrylate, isopropyl acrylate, butyl acrylate, isobutyl acrylate, methylheptyl acrylate, dodecyl acrylate, stearyl acrylate, 2-hydroxyethyl acrylate, 2-hydroxypropyl acrylate, 4-hydroxybutyl acrylate, glycidyl acrylate, N,N-dimethylethyl acrylate, N,N-diethylethyl acrylate, N,N-diethylpropyl acrylate, N,N-dimethylbutyl acrylate, and N,N-diethylbutyl acrylate; and
further preferably, an acid value of the alkali-soluble resin is 120-250 mg KOH/g, more preferably, a weight-average molecular weight is 50000-120000, a molecular weight distribution is 1.3-2.5, and further preferably, a polymerization conversion rate is greater than or equal to 97%.

23. (canceled)

24. A photosensitive dry film, comprising a dry film resist layer, and a support layer and a protective layer located on two sides of the dry film resist layer, wherein the dry film resist layer contains the dry film resist according to claim 16.

25. A copper clad laminate, provided with the dry film resist according to claim 16.
